(19) United States
(12) Patent Application Publication
(10) Pub. No.: US 2010/0016313 A1
(43) Pub. Date: Jan. 21, 2010

(54) INTESTINAL ALKALINE PHOSPHATASE MODULATORS AND USES THEREOF

(75) Inventors: Jose Luis Millan, San Diego, CA (US); Sonoko Narisawa, San Diego, CA (US); Eduard Sergienko, San Diego, CA (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: Burnham Institute for Medical Research, La Jolla, CA (US)

(21) Appl. No.: 12/468,476

(22) Filed: May 19, 2009

Related U.S. Application Data
(60) Provisional application No. 61/054,326, filed on May 19, 2008.

(51) Int. Cl. A61K 31/454; A61K 31/415; A61K 31/4162; A61K 31/4196; A61K 31/41; A61K 31/4245; A61K 31/426; A61K 31/5377; A61K 31/497; A61K 31/53; C12N 5/02; A61P 31/04

(52) U.S. Cl. 514/235.2; 514/406; 514/383; 514/381; 514/364; 514/365; 514/323; 514/254.05; 514/241; 435/375

(57) ABSTRACT

Disclosed are modulators, i.e., activators and inhibitors, of Intestinal Alkaline Phosphatase (IAP). Also disclosed are methods for treating bacterial infections of the intestinal tract and methods for maintaining the health of the intestinal tract using IAP activators. Further disclosed are methods to assist in weight gain of emaciated patients and those having reduced or negligible fat absorption using IAP inhibitors.

### CROSS-REFERENCE TO RELATED APPLICATIONS

This application claims benefit of U.S. Provisional Application No. 61/054,326, filed May 19, 2008. Application No. 61/054,326, filed May 19, 2008, is hereby incorporated herein by reference in its entirety.

### STATEMENT REGARDING FEDERALLY SPONSORED RESEARCH

This invention was made with government support under Grant ROI DE 012889 awarded by the National Institutes of Health. The government has certain rights in the invention.

### FIELD

Disclosed are modulators, i.e., activators and inhibitors, of Intestinal Alkaline Phosphatase (IAP). Also disclosed are methods for treating bacterial infections of the intestinal tract and methods for maintaining the health of the intestinal tract using IAP activators. Further disclosed are methods to assist in weight gain of emaciated patients and those having reduced or negligible fat absorption using IAP inhibitors.

### BACKGROUND

The mammalian gut mucosa provides a barrier to luminal microbes and toxins while still allowing for digestion and absorption of dietary nutrients that are essential for survival. Impairment of the gut mucosa can often have severe consequences. Under conditions of starvation and disease, the gut barrier can be become damaged, leading to morbidity and even mortality. Diseases and trauma of the gastrointestinal tract often severely impair the gut barrier. Neurologic diseases, muscular diseases, and diabetes can lead to abnormal muscular activity in the intestine causing bacterial overgrowth and inflammation of the gastrointestinal tract. Trauma resulting in physical intestinal obstruction, such as scarring, can also impair the gut barrier. Crohn's disease is an example of an especially debilitating gastrointestinal disease that affects between 400,000 and 600,000 people in North America alone. Crohn's disease patients can suffer from fistula, rectal bleeding, constipation, fever, rheumatologic disease, and malnutrition. Because Crohn's disease can severely damage the gastrointestinal tract, the disease can lead to fatal illnesses such as cancer of the small and large intestines. Needed therefore are compositions and methods to protect gut mucosa with barrier dysfunction.

### BRIEF SUMMARY

In accordance with the purpose of this invention, as embodied and broadly described herein, this invention relates to modulators of Intestinal Alkaline Phosphatase. The activators can be used as a method for suppressing gut mucosal atrophy during trophic enteral feeding thereby maintaining the intestinal mucosa as a barrier to luminal microbes and toxins. The IAP activators are also useful for suppressing bacterial colonization in the gut. The activators can further provide a method for detoxifying bacterial lipopolysaccharide (LPS). The inhibitors can be used as a method for increasing fat absorption in the gut of patients needing increased fat absorption. In addition, the inhibitors can be used to increase the fat absorption, and hence the body weight, of mammals having IAP expressed in the intestinal tract.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows genomic organization of the murine alkaline phosphatase (AP) loci. The mouse tissue-nonspecific AP (TNAP) gene (Akp2) is located at 4D3 in chromosome 4. It stretches for 55 kb and consists of 12 exons and 11 introns including an alternate exon (exon 1b), located ˜30 kb downstream of exon 1a. The mouse tissue-specific AP (TSAP) genes (Akp3, Akp5, Akp6, and the Akp-ps1 pseudogene) are closely linked at 1C5 site in chromosome 1. The size of each TSAP genei is ˜3.5 kb and they contain 11 exons and 10 introns. The direction of the Akp3 gene and the Akp-ps1 pseudogene is opposite to that of Akp5 and Akp6 genes. In the active AP genes, translation starts from the ATP site in the exon 2 and ends at the stop codon within the exon 11. Sequence numbers indicated beneath each gene are the actual location in the chromosome.

FIG. 2 shows expression of Akp3, Akp5, and Akp6 in the murine gut under normal feeding, and high-fat feeding. Shown is Northern blot analysis of each intestinal segment, isolated as indicated in the picture, for expression of Akp3, Akp5, and Akp6 mRNA. Akp3 is exclusively expressed in the duodenum. Akp5 is expressed in the duodemum, jejunum, and ileum, and its expression is not affected by high-fat feeding. Akp6 expression is strong in the duodenum and also detectable in jejumum and ileum. The jejunal-ileal expression is particularly increased in Akp3^{-/-} animals after corn oil administration or long-term high-fat feeding.

FIG. 3 shows postnatal expression of Akp3, Akp5, and Akp6 mRNA in the mouse gut. Total RNA was extracted from the entire small intestine of postnatal WT mice from day 2 until day 28 as indicated and run on a Northern blot. Mice were weaned at day 18.

FIG. 4 shows post translational modifications of gIAP and EAP in the jejunum further modulate the catalytic properties of these intestinal phosphatases. Small intestines of 2- or 10-day-old WT mice were divided into 4 segments (upper to lower, segments 1, 2, 3, and 4), and in the case of e18.5 embryo, the entire small intestine were used. Protein extract (50 μg) was loaded in each lane of 8-16% acrylamide Tris-glycine gel. The same amount of recombinant gIAP was loaded as a standard between the 2 blots stained with anti-gIAP antibody. Illeum samples from Akp5−/− and WT mice and recombinant EAP protein were loaded using the same conditions. Enzyme immunoassay (EIA) was performed on butanol extracts from each intestinal segment as indicated. Extracts from segment 1 were treated with endo-β-galactosidase.

FIG. 5A shows IAP blocks LPS-activated NF-κB nuclear translocation. HT-29 parental cell, transfectant with empty vector and IAP-overexpressing cells were exposed LPS (+ or −), then fixed and stained for immunoflorescence studies. Staining with antibodies for RelA/p65 (part of the NF-κB complex translocated into the nucleus) and DAPI (cell nucleus). Only the IAP-overexpressing cells were able to block the effects of LPS, preventing NF-κB nuclear translocation.

FIG. 5B shows IAP protects the cell from LPS exposure. Parental and IAP-expressing IEC-6 cells were exposed to LPS at varying concentrations. NF-κB-Luc activity was determined as the readout for the cellular effects of LPS. Data refer to mean±SD.

FIG. 5C shows IAP specifically blocks LPS activation of the NF-κB pathway in EIC-6 cells. Western blotting was performed with a specific antibody to IκBα phosphorylation, a critical step in the NF-κB pathway. IκBα did not become phosphorylated in the case of the IAP-over-expressing cells exposed to LPS. The β-actin staining was used to confirm the relative amounts of protein in each sample.

FIG. 6 shows LPS dephosphorylating activity measured by LPS/malachite green assay. FIG. 6A shows biological activity is present in the transfected, but not parent HT-29 cells, the magnitude greatest in the cell lysate>membrane>media (all significant, p<0.01). There was not statistically significant difference in LPS dephosphorylating activity in the cytosol between the transformant and parent cells. FIG. 6B shows the LPS dephosphorylating activity is compared in the endogenous (butyrate-treated) and ectopically-produced (transfected cells) conditions. The increases in the lysates became significant (p<0.01) at 12 and 24 hours of butyrate exposure and in the media at 24 hours. Data are presented as mean±SD.

FIG. 7A shows pNPPase assay. Duodenum mucosa lysate from WT and Akp3^{-/-} mice which were fed (n=5), fasted (starved for 2 days, n−5), and refed (starved for 2 days, n=4) were measured for alkaline phosphatase activity. Starvation causes significant decrease in the WT animals, down to levels similar to those in the Akp3−/− mice. Refeeding stimulates IAP expression in the WT mice. Starvation and refeeding appear to have minimal effect on IAP expression in the Akp3^{-/-} mice. Significance: * is p<0.05, comparing fasted to the fed and refed WT animals. AP levels in the knockout animals were significantly lower than those in the WT animals.

FIG. 7B shows LPS/malachite green assay. A similar pattern was seen in the LPS dephosphorylating activity with the fed, fasted, and refed WT and knockout groups. Starvation dramatically reduced the LPS dephosphorylating ability of the WT type animal, while refeeding returned it to normal levels. Significance: * is p<0.05, comparing fasted to the fed and refed WT mice. Phosphate levels in Akp3^{-/-} are significantly lower than those in WT animals.

FIG. 8 shows dose response curve of compound MLS-0091968 (F5) for IAP, AKP3, AKP5, and AKP6 inhibition. Note positive number means positive inhibition.

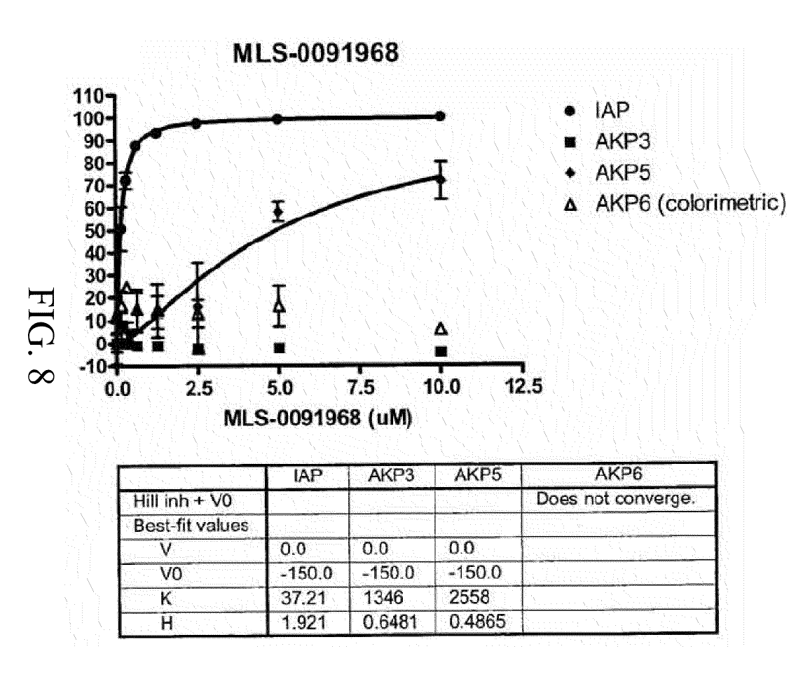

FIG. 9 shows dose response curve of compound MLS-0067142 (F8) for IAP, AKP3, AKP5, and AKP6 inhibition. Note positive number means positive inhibition.

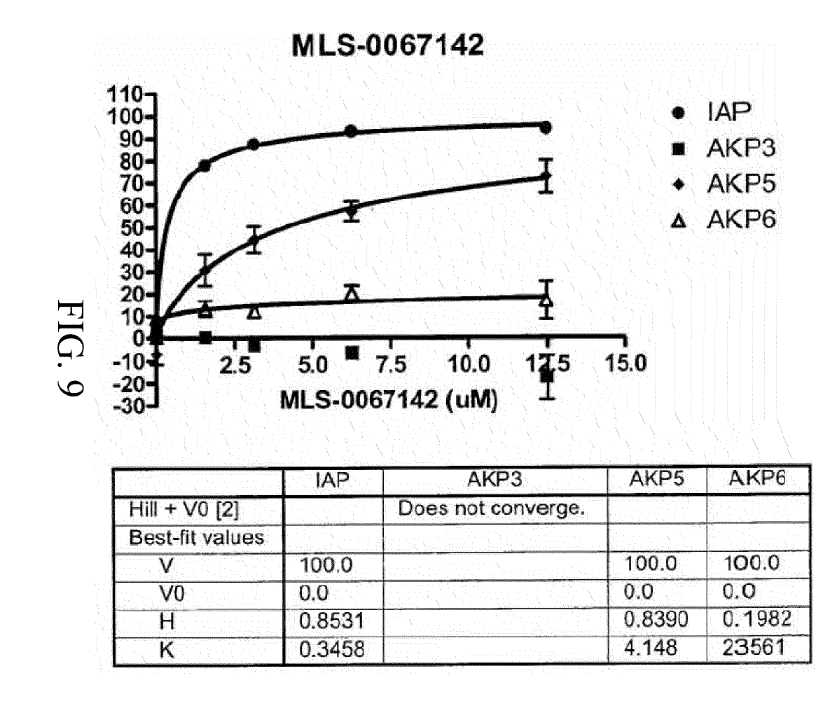

FIG. 10 shows dose response curve of compound MLS-0091976 (F1) for IAP, AKP3, AKP5, and AKP6 inhibition. Note positive number means positive inhibition.

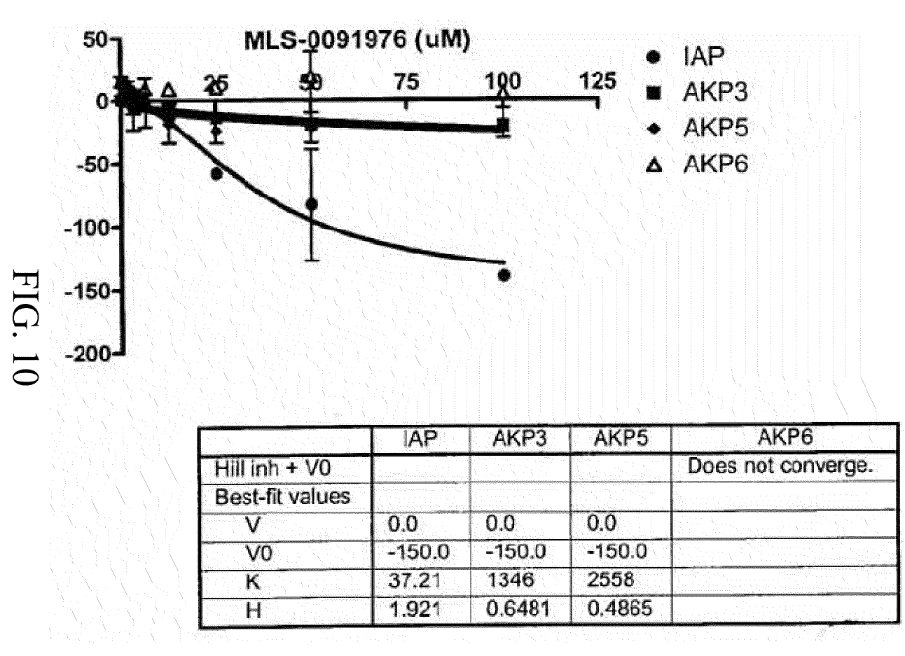

FIG. 11 shows dose response curve of compound MLS-0111632 (B2) for IAP, AKP3, AKP5, and AKP6 inhibition. Note positive number means positive inhibition.

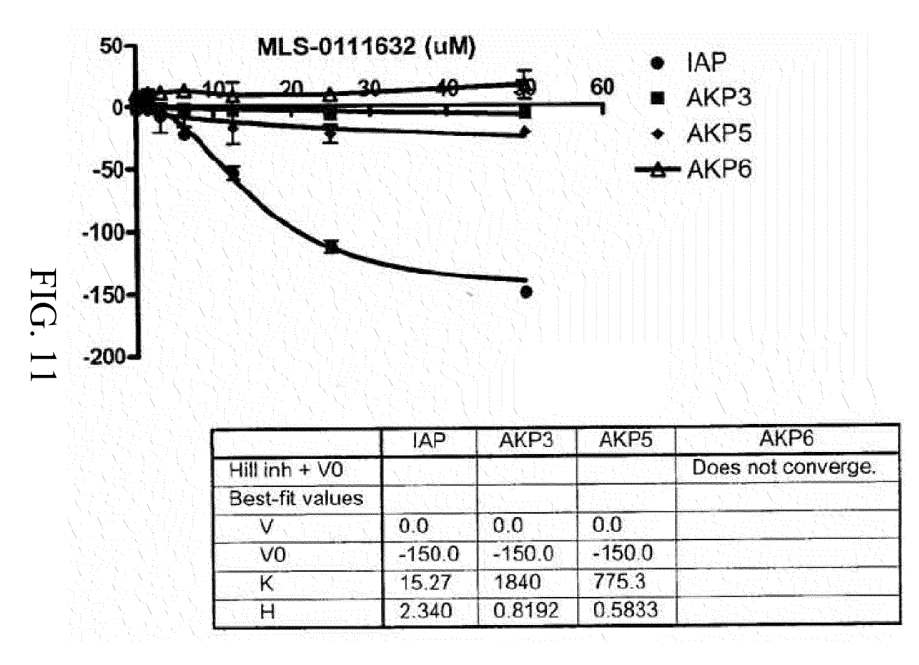

FIG. 12 shows dose response curve of compound MLS-0111581 (E4) for IAP, AKP3, AKP5, and AKP6 inhibition. Note positive number means positive inhibition.

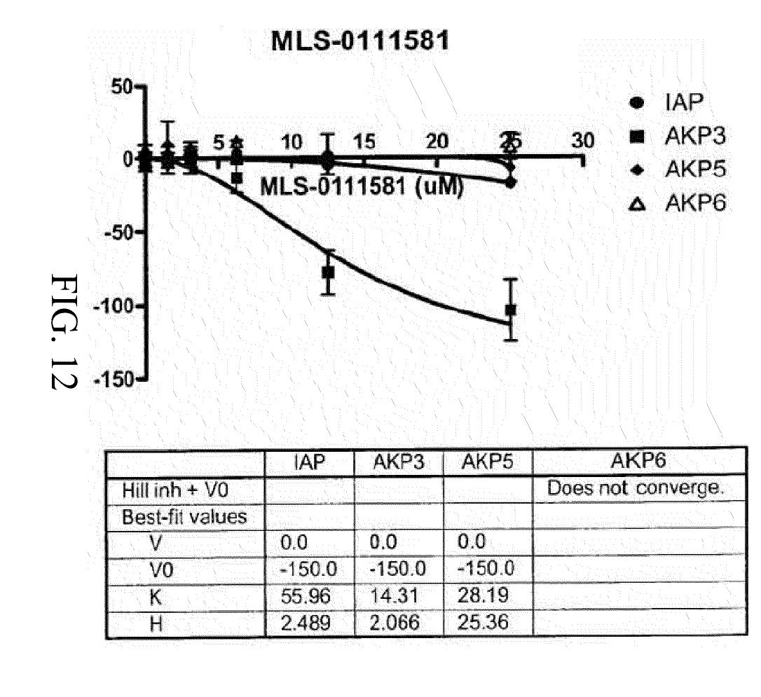

FIG. 13 illustrates the IAP assay procedure using CDP-Star.

FIG. 14 illustrates the screening strategy for identifying IAP activators.

FIG. 15 shows that IAP protects the mice from gut bacterial translocation. (A) Direct gut I/R. WT and IAP KO mice were exposed to 45 min of superior mesenteric ligation clamping followed by varying times of reperfusion. Sham laparotomy and no intervention were used as controls. Mesenteric tissues were harvested, and bacterial counts in the nodes were determined. Data are based on experiments repeated on multiple occasions, n=4 for no surgery, sham laparotomy, O and 4-h groups; n=7 for 24-, 48-, and 120-h groups. *, P<0.05, comparing the values with previous time points. **, P<0.05, comparing KO with WT mice. (B) Remote trauma. After hind-limb I/R, mesenteric tissues were harvested, and bacterial counts in the nodes were determined. Sham mice were used for control purposes in all experiments. *, P<0.05, comparing KO with WT mice. Data in this figure are presented as mean±SEM.

FIG. 16 shows the colitis associated cancer mode. The time course in weeks is shown below the structures for AOM and DSS.

FIG. 17 shows macroscopic colon tumors after 9 weeks of AOM/DSS treatment. AA indicates Ets2^{A72/A72} mice. Error bars show the standard deviation. Difference was highly significant by T-test (P=0.003).

FIG. 18 shows the tumor development after AOM/DSS treatment. (A) tumor incidence from the second trial analyzed 19 weeks after AOM injection. (B) average number of tumors/mouse; (C) average tumor weight. Differences in tumor weight were not significant. (P=0.097). Differences in tumor number/mouse in both trials were highly significant.

### DETAILED DESCRIPTION

The disclosed method and compositions may be understood more readily by reference to the following detailed description of particular embodiments and the Example included therein and to the Figures and their previous and following description.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a compound is disclosed and discussed and a number of modifications that can be made to a number of molecules including the compound are discussed, each and every combination and permutation of compound and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the method and compositions described herein. Such equivalents are intended to be encompassed by the following claims.

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

### A. COMPOSITIONS

This application is related to the subject matter of U.S. patent application Ser. No. 11/576,251, filed Mar. 28, 2007, the contents of which are incorporated herein by reference.

1. IAP Modulators

Provided herein are modulators of intenstinal alkaline phosphatase (IAP) that can be used, for example, as mucosal defense against bacterial invasion. In some aspects, the IAP is human IAP. Table 1 provides the nomenclature of the different alkaline phosphatase isozymes disclosed herein.

TABLE 1
Alkaline Phosphatase Isozymes

| Species | Gene | Protein | Common names in use |
|---|---|---|---|
| Human | ALPL | TNAP | Tissue-nonspecific alkaline phosphatase; |
| | | | TNSALP; “liver-bone-kidney type” AP |
| | ALPP | PLAP | Placental alkaline phosphatase; PLALP |
| | ALPP2 | GCAP | Germ cell alkaline phosphatase; GCALP |
| | ALPI | IAP | Intestinal alkaline phosphatase; IALP |
| Mouse | Akp2 | TNAP | Tissue-nonspecific alkaline phosphatase; |
| | | | TNSALP; “liver-bone-kidney type” AP |
| | Akp3 | dIAP | Duodenal Intestinal alkaline phosphatase; |
| | | | IALP |
| | Akp5 | EAP | Embryonic alkaline phosphatase |
| | Akp-ps1 | N/a | AP Pseudogene, pseudoAP |
| | Akp6 | gIAP | Global Intestinal alkaline phosphatase |
| Rat | Alp1 | TNAP | Tissue-nonspecific alkaline phosphatase; |
| | | | TNSALP; “liver-bone-kidney type” AP |
| | Alpi | IAPI | Intestinal alkaline phosphatase I |
| | Alpi2 | IAPII | Intestinal alkaline phosphatase II |

The Intestinal Alkaline Phosphatase modulators of the present disclosure are arranged into several categories to assist the formulator in applying a rational synthetic strategy for the preparation of analogs that are not expressly exemplified herein. The arrangement into categories does not imply increased or decreased efficacy for any of the Intestinal Alkaline Phosphatase modulators described herein.

One category of Intestinal Alkaline Phosphatase modulators relates to compounds having the formula:

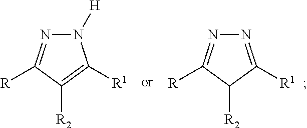

wherein R and R¹ are each independently chosen from:
i) hydrogen;
ii) substituted or unsubstituted C₆, C₁₀, or C₁₄ aryl; or
iii) —C(O)R⁴, wherein R⁴ is a hydrocarbyl unit;
   R and R² can be taken together to form a fused ring system having the formula:

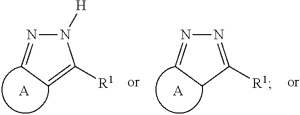

R¹ and R² can be taken together to form a fused ring system having the formula:

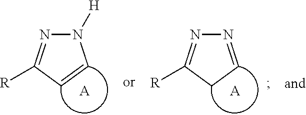

A is one or more substituted or unsubstituted cycloalkyl, aryl, heterocyclic, or heteroaryl rings having from 3 to 14 carbon atoms and from 1 to 5 heteroatoms chosen from oxygen, nitrogen, sulfur, or combinations thereof.

One aspect of this category relates to Intestinal Alkaline Phosphatase modulators having the formula:

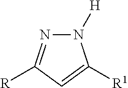

wherein R is a unit having the formula —C(O)R⁴ and R¹ is substituted or unsubstituted C₆ aryl (phenyl) or R¹ is a unit having the formula —C(O)R⁴ and R is substituted or unsubstituted C₆ aryl (phenyl). One embodiment of this aspect relates to modulators having the formula:

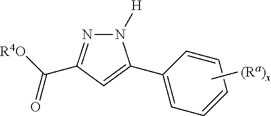

wherein R⁴ is chosen from:
a) substituted or unsubstituted C₁-C₁₀ linear, branched, or cyclic alkyl;
b) —OR⁵ wherein R⁵ is chosen from:
i) hydrogen;
ii) substituted or unsubstituted C₁-C₄ linear or branched alkyl; wherein each substitution on the alkyl chain is independently chosen from:
i) halogen; and
ii) —[C(R^{7a})(R^{7b})]_{w}C(O)R⁶;
   R⁶ is hydroxy, C₁-C₄ linear or branched alkoxy, or —N(R^{8a})(R^{8b}), each R^{8a} and R^{8b} is independently chosen from hydrogen or C₁-C₁₀ linear, branched or cyclic alkyl;
iii) —[C(R^{7a})(R^{7b})]_{w}N(R^{9a})(R^{9b});
   each R^{9a} and R^{9b} is independently chosen from hydrogen or C₁-C₁₀ linear, branched or cyclic alkyl; or R^{9a} and R^{9b} can be taken together to form a ring having from 3 to 7 atoms; each R^{7a} and R^{7b} is independently hydrogen or C₁-C₄ linear or branched alkyl; the index w is an integer from 0 to 5.

Each R^{a} represents from 1 to 5 optionally present substitutions for a hydrogen atom on the phenyl ring, as such the index x is an integer from 0 to 5. Each R^{a} is independently chosen from
i) C₁-C₁₂ substituted or unsubstituted linear, branched, or cyclic alkyl;
ii) C₂-C₁₂ substituted or unsubstituted linear, branched, or cyclic alkenyl;
iii) C₂-C₁₂ substituted or unsubstituted linear or branched alkynyl;
iv) C₆ or C₁₀ substituted or unsubstituted aryl;
v) C₁-C₉ substituted or unsubstituted heterocyclic;
vi) C₁-C₁₁ substituted or unsubstituted heteroaryl;
vii) —[C(R^{26a})(R^{26b})]ₓOR¹⁰;
   R¹⁰ is chosen from:
   a) —H;
   b) C₁-C₁₂ substituted or unsubstituted linear, branched, or cyclic alkyl;
   c) C₆ or C₁₀ substituted or unsubstituted aryl or alkylenearyl;
   d) C₁-C₉ substituted or unsubstituted heterocyclic;
   e) C₁-C₁₁ substituted or unsubstituted heteroaryl;
viii) —[C(R^{26a})(R^{26b})]ₙN(R^{11a})(R^{11b});
   R^{11a} and R^{11b} are each independently chosen from:
   a) —H;
   b) —OR¹²;
   R¹² is hydrogen or C₁-C4 linear alkyl;
   c) C₁-C₁₂ substituted or unsubstituted linear, branched, or cyclic alkyl;
   d) C₆ or C₁₀ substituted or unsubstituted aryl;
   e) C₁-C₉ substituted or unsubstituted heterocyclic;
   f) C₁-C₁₁ substituted or unsubstituted heteroaryl; or
   g) R^{11a} and R^{11b} can be taken together to form a substituted or unsubstituted ring having from 3 to 10 carbon atoms and from 0 to 3 heteroatoms chosen from oxygen, nitrogen, and sulfur;
ix) —[C(R^{26a})(R^{26b})]ₙC(O)R¹³;
   R¹³ is:
   a) C₁-C₁₂ substituted or unsubstituted linear, branched, or cyclic alkyl;
   b) —OR¹⁴;
   R¹⁴ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl;
   c) —N(R^{15a})(R^{15b});
   R^{15a} and R^{15b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{15a} and R^{15b} can be taken together to form a substituted or unsubstituted ring having from 3 to 10 carbon atoms and from 0 to 3 heteroatoms chosen from oxygen, nitrogen, and sulfur;
x) —[C(R^{24a})(R^{24b})]ₙOC(O)R¹⁶;
   R¹⁶ is:
   a) C₁-C₁₂ substituted or unsubstituted linear, branched, or cyclic alkyl;
   b) —N(R^{17a})(R^{17b});
   R^{17a} and R^{17b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{17a} and R^{17b} can be taken together to form a substituted or unsubstituted ring having from 3 to 10 carbon atoms and from 0 to 3 heteroatoms chosen from oxygen, nitrogen, and sulfur;
xi) —[C(R^{24a})(R^{24b})]ₙNR¹⁸C(O)R¹⁹;
   R¹⁸ is:
   a) —H; or
   b) C₁-C₄ substituted or unsubstituted linear, branched, or cyclic alkyl;
   R¹⁹ is
   a) C₁-C₁₂ substituted or unsubstituted linear, branched, or cyclic alkyl;
   b) —N(R^{20a})(R^{20b});
   R^{20a} and R^{20b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{20a} and R^{20b} can be taken together to form a substituted or unsubstituted ring having from 3 to 10 carbon atoms and from 0 to 3 heteroatoms chosen from oxygen, nitrogen, and sulfur;
xii) —[C(R^{24a})(R^{24b})]ₙCN;
xiii) —[C(R^{24a})(R^{24b})]ₙNO₂;
xiv) —[C(R^{24a})(R^{24b})]ₙR²¹;
   R²¹ is C₁-C₁₀ linear, branched, or cyclic alkyl substituted by from 1 to 21 halogen atoms chosen from —F, —Cl, —Br, or —I;
xv) —[C(R^{24a})(R^{24b})]ₙSO₂R²²;
   R²² is hydrogen, hydroxyl, substituted or unsubstituted C₁-C₄ linear or branched alkyl; substituted or unsubstituted C₆, C₁₀, or C₁₄ aryl; C₇-C₁₅ alkylenearyl; C₁-C₉ substituted or unsubstituted heterocyclic; or C₁-C₁₁ substituted or unsubstituted heteroaryl;
ii) two R^{a} units on the same carbon atom can be taken together to form a unit chosen from ═O, ═S, or ═NR²³;
   R²³ is hydrogen, hydroxyl, C₁-C₄ linear or branched alkyl, or C₁-C₄ linear or branched alkoxy;
      R^{24a} and R^{24b} are each independently hydrogen or C₁-C₄ alkyl;
      the index n is an integer from 0 to 5.

The R^{a} units disclosed herein can be further substituted by one or more organic radicals independently chosen from:
i) C₁-C₁₂ linear, branched, or cyclic alkyl, alkenyl, and alkynyl;
ii) substituted or unsubstituted C₆ or C₁₀ aryl;
iii) substituted or unsubstituted C₆ or C₁₀ alkylenearyl;
iv) substituted or unsubstituted C₁-C₉ heterocyclic rings;
v) substituted or unsubstituted C₁-C₉ heteroaryl rings;
vi) —(CR^{102a}R^{102b})_{z}OR¹⁰¹;
vii) —(CR^{102a}R^{102b})_{z}C(O)R¹⁰¹;
viii) —(CR^{102a}R^{102b})_{z}C(O)OR¹⁰¹;
ii) —(CR^{102a}R^{102b})_{z}C(O)N(R¹⁰¹)₂;
ix) —(CR^{102a}R^{102b})_{z}N(R¹⁰¹)₂;
xi) halogen;
xii) —(CR^{102a}R^{102b})_{z}CN;
xiii) —(CR^{102a}R^{102b})_{z}NO₂;
xiv) —CHⱼXₖ; wherein X is halogen, the index j is an integer from 0 to 2, j+k=3;
xv) —(CR^{102a}R^{102b})_{z}SR¹⁰¹;
xvi) —(CR^{102a}R^{102b})_{z}SO₂R¹⁰¹; and
xvii) —(CR^{102a}R^{102b})_{z}SO₃R¹⁰¹;
   wherein each R¹⁰¹ is independently hydrogen, substituted or unsubstituted C₁-C₄ linear, branched, or cyclic alkyl, phenyl, benzyl, heterocyclic, or heteroaryl; or two R¹⁰¹ units can be taken together to form a ring comprising 3-7 atoms; R^{102a} and R^{102b} are each independently hydrogen or C₁-C₄ linear or branched alkyl; the index z is from 0 to 4.

Non-limiting examples of R units according to this embodiment includes units chosen from:
i) —CO₂H;
ii) —CO₂CH₃;
iii) —CO₂CHCH₃;
iv) —CO₂CF₃;
v) —CONHCH₃; and
vi) —CON(CH₃)₂.

Non-limiting examples of R¹ units according to this embodiment include the following:

Halogen substituted phenyl, for example, 2-fluorophenyl, 3-fluorophenyl, 4-fluorophenyl, 2,3-difluorophenyl, 2,4-difluorophenyl, 2,5-difluorophenyl, 2,6-difluorophenyl, 3,4-difluorophenyl, 3,5-difluorophenyl, 2-chlorophenyl, 3-chlorophenyl, 4-chlorophenyl, 2,3-dichlorophenyl, 2,4-dichlorophenyl, 2,5-dichlorophenyl, 2,6-dichlorophenyl, 3,4-dichlorophenyl, 3,5-dichlorophenyl, 2-bromophenyl, 3-bromophenyl, 4-bromophenyl, 2,3-dibromophenyl, 2,4-dibromophenyl, 2,5-dibromophenyl, 2,6-dibromophenyl, 3,4-dibromophenyl, and 3,5-dibromophenyl.

Alkyl substituted phenyl, for example, 2-methylphenyl, 3-methylphenyl, 4-methylphenyl, 2,3-dimethylphenyl, 2,4-dimethylphenyl, 2,5-dimethylphenyl, 2,6-dimethylphenyl, 3,4-dimethylphenyl, 3,5-dimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 3,5-diethylphenyl, 2-n-propylphenyl, 3-n-propylphenyl, 4-n-propylphenyl, 2,3-di-n-propylphenyl, 2,4-di-n-propylphenyl, 2,5-di-n-propylphenyl, 2,6-di-n-propylphenyl, 3,4-di-n-propylphenyl, 3,5-di-n-propylphenyl, 2-iso-propylphenyl, 3-iso-propylphenyl, 4-iso-propylphenyl, 2,3-di-iso-propylphenyl, 2,4-dii-so-propylphenyl, 2,5-di-iso-propylphenyl, 2,6-di-iso-propylphenyl, 3,4-di-iso-propylphenyl, and 3,5-di-iso-propylphenyl.

Alkoxy substituted phenyl, for example, 2-methoxyphenyl, 3-methoxyphenyl, 4-methoxyphenyl, 2,3-dimethoxyphenyl, 2,4-dimethoxyphenyl, 2,5-dimethoxyphenyl, 2,6-dimethoxyphenyl, 3,4-dimethoxyphenyl, 3,5-dimethoxyphenyl, 2-ethoxyphenyl, 3-ethoxyphenyl, 4-ethoxyphenyl, 2,3-diethoxyphenyl, 2,4-diethoxyphenyl, 2,5-diethoxyphenyl, 2,6-diethoxyphenyl, 3,4-diethoxyphenyl, 3,5-diethoxyphenyl, 2-propoxyphenyl, 3-propoxyphenyl, 4-propoxyphenyl, 2,3-dipropoxyphenyl, 2,4-dipropoxyphenyl, 2,5-dipropoxyphenyl, 2,6-dipropoxyphenyl, 3,4-dipropoxyphenyl, and 3,5-dipropoxyphenyl.

Hydroxy, nitro, cyano, thiol, and amino substituted phenyl, for example, 2-hydroxyphenyl, 3-hydroxyphenyl, 4-hydroxyphenyl, 2,3-dihydroxyphenyl, 2,4-dihydroxyphenyl, 2,5-dihydroxyphenyl, 2,6-dihydroxyphenyl, 3,4-dihydroxyphenyl, 3,5-dihydroxyphenyl, 2-nitrophenyl, 3-nitrophenyl, 4-nitrophenyl, 2,3-dinitrophenyl, 2,4-dinitrophenyl, 2,5-dinitrophenyl, 2,6-dinitrophenyl, 3,4-dinitrophenyl, 3,5-dinitrophenyl, 2-cyanophenyl, 3-cyanophenyl, 4-cyanophenyl, 2,3-dicyanophenyl, 2,4-dicyanophenyl, 2,5-dicyanophenyl, 2,6-dicyanophenyl, 3,4-dicyanophenyl, 3,5-dicyanophenyl, 2-thiophenyl, 3-thiophenyl, 4-thiophenyl, 2,3-dithiophenyl, 2,4-dithiophenyl, 2,5-dithiophenyl, 2,6-dithiophenyl, 3,4-dithiophenyl, 3,5-dithiophenyl, 2-aminophenyl, 3-aminophenyl, 4-aminophenyl, 2,3-diaminophenyl, 2,4-diaminophenyl, 2,5-diaminophenyl, 2,6-diaminophenyl, 3,4-diaminophenyl, and 3,5-diaminophenyl.

Trifluoromethyl and sulfoxy substituted phenyl, for example, 2-trifluoromethylphenyl, 3-trifluoromethylphenyl, 4-trifluoromethylphenyl, 2,3-ditrifluoromethylphenyl, 2,4-ditrifluoromethylphenyl, 2,5-ditrifluoromethylphenyl, 2,6-ditrifluoromethylphenyl, 3,4-ditrifluoromethylphenyl, 3,5-ditrifluoromethylphenyl, 2-sulfoxyphenyl, 3-sulfoxyphenyl, 4-sulfoxyphenyl, 2,3-disulfoxyphenyl, 2,4-disulfoxyphenyl, 2,5-disulfoxyphenyl, 2,6-disulfoxyphenyl, 3,4-disulfoxyphenyl, and 3,5-disulfoxyphenyl.

One iteration of this embodiment relates to compounds having the formula:

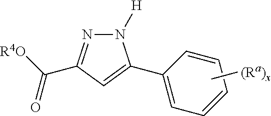

wherein R⁴ is —OR⁵, R⁵ is chosen from:
i) hydrogen; or
ii) substituted or unsubstituted C₁-C₄ linear or branched alkyl; each substitution is independently chosen from:
   a) —[C(R^{7a})(R^{7b})]_{w}C(O)R⁶; R⁶ is hydroxy, C₁-C₄ linear or branched alkoxy, or —N(R^{8a})(R^{8b}), each R^{8a} and R^{8b} is independently chosen from hydrogen or C₁-C₁₀ linear, branched or cyclic alkyl;
   b) —[C(R^{7a})(R^{7b})]_{w}N(R^{9a})(R^{9b}); each R^{9a} and R^{9b} is independently chosen from hydrogen or C₁-C₁₀ linear, branched or cyclic alkyl; or R^{9a} and R^{9b} can be taken together to form a ring having from 3 to 7 atoms;
   each R^{7a} and R^{7b} is independently hydrogen or C₁-C₄ linear or branched alkyl; the index w is an integer from 0 to 5; and
      each R^{a} is chosen from:
   i) C₁-C₄ linear or branched alkyl;
   ii) C₁-C₄ linear or branched alkoxy;
   iii) —OH;
   iv) —F;
   v) —Cl;
   vi) —Br;
   vii) —NO₂;
   viii) —NH₂; and
   ix) —CF₃;
      the index x is an integer from 0 to 5, and the integer w is from 0 to 2.

Non-limiting examples of this iteration include modulators having the general formula:
i) 2-oxoalkyl 5-(substituted or unsubstituted phenyl)-1H-pyrazole-3-carboxylates:

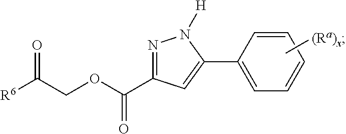

wherein R⁶ is chosen from methyl (C₁), ethyl (C₂), n-propyl (C₃), iso-propyl (C₃), n-butyl (C₄), sec-butyl (C₄), iso-butyl (C₄), and tert-butyl (C₄), for example, compounds having the formula:
a) 2-oxopropyl 5-phenyl-1H-pyrazole-3-carboxylate

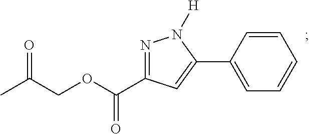

b) 2-oxopropyl 5-(4-bromophenyl)-1H-pyrazole-3-carboxylate

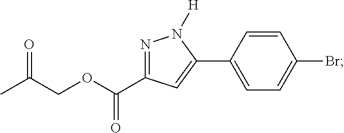

c) 3-methyl-2-oxobutyl 5-(4-bromophenyl)-1H-pyrazole-3-carboxylate

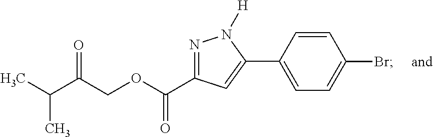

d) 3,3-dimethyl-2-oxobutyl 5-(4-bromophenyl)-1H-pyrazole-3-carboxylate

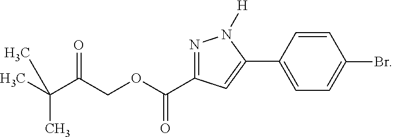

ii) N-alkylamino-oxoalkyl 5-(substituted or unsubstituted phenyl)-1H-pyrazole-3-carboxylates:

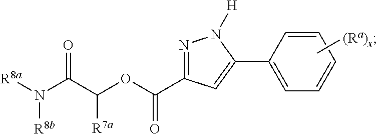

wherein R^{7a} is chosen from hydrogen, methyl (C₁), or ethyl (C₂); R^{7b} is hydrogen R^{8b} is hydrogen and R^{8a} is chosen from hydrogen, methyl (C₁), ethyl (C₂), n-propyl (C₃), iso-propyl (C₃), cyclopropyl (C₃), n-butyl (C₄), sec-butyl (C₄), iso-butyl (C₄), tert-butyl (C₄), cyclobutyl (C₄), cyclopentyl (C₅), or cyclohexyl (C₆). For example, compounds having the formula:
a) 1-(methylamino)-1-oxopropan-2-yl 5-phenyl-1H-pyrazole-3-carboxylate

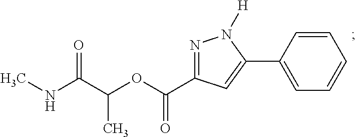

b) 2-(methylamino)-2-oxoethyl 5-(4-bromophenyl)-1H-pyrazole-3-carboxylate

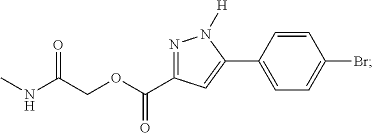

c) 2-(methylamino)-2-oxoethyl 5-(4-bromophenyl)-1H-pyrazole-3-carboxylate

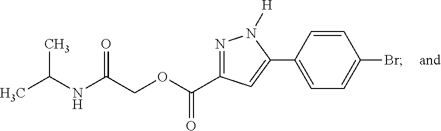

d) 1-(tert-butylamino)-1-oxopropan-2-yl 5-(4-bromophenyl)-1H-pyrazole-3-carboxylate

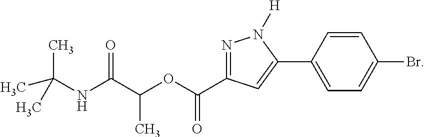

iii) N,N-dialkylamino-oxoalkyl 5-(substituted or unsubstituted phenyl)-1H-pyrazole-3-carboxylates:

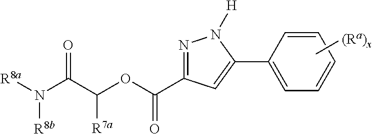

wherein R^{7a} is chosen from hydrogen, methyl (C₁), or ethyl (C₂); R^{7b} is hydrogen; and R^{8a} and R^{8b} are each independently chosen from hydrogen, methyl (C₁), ethyl (C₂), n-propyl (C₃), iso-propyl (C₃), cyclopropyl (C₃), n-butyl (C₄), sec-butyl (C₄), iso-butyl (C₄), tert-butyl (C₄), cyclobutyl (C₄), cyclopentyl (C₅), or cyclohexyl (C₆). For example, 2-[cyclohexyl(methyl)-amino]-2-oxoethyl 5-(4-bromophenyl)-1H-pyrazole-3-carboxylate having the formula:

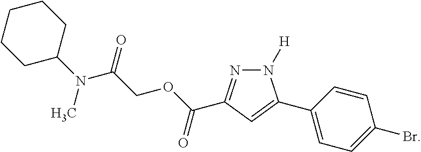

Non-limiting examples of this embodiment include:
i) 5-(2-chlorophenyl)-1H-pyrazole-3-carboxylic acid

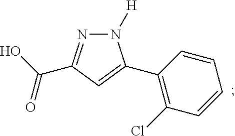

ii) 5-(4-hydroxyphenyl)-1H-pyrazole-3-carboxylic acid

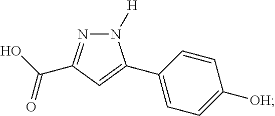

iii) 5-(2-hydroxyphenyl)-1H-pyrazole-3-carboxylic acid

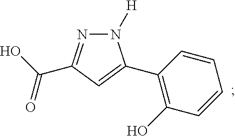

iv) 5-(4-chlorophenyl)-1H-pyrazole-3-carboxylic acid

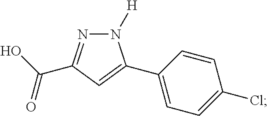

v) 5-(4-methoxyphenyl)-1H-pyrazole-3-carboxylic acid

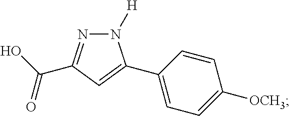

vi) 5-(4-methylphenyl)-1H-pyrazole-3-carboxylic acid

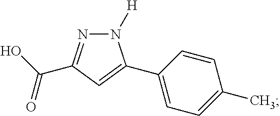

vii) 5-(4-aminophenyl)-1H-pyrazole-3-carboxylic acid

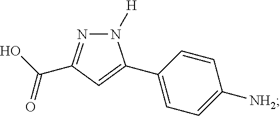

viii) ethyl 5-[3-(trifluoromethyl)phenyl]-1H-pyrazole-3-carboxylate

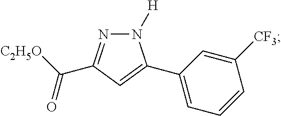

ix) methyl 5-(4-bromophenyl)-1H-pyrazole-3-carboxylate

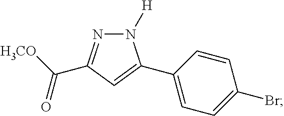

x) methyl 5-phenyl-1H-pyrazole-3-carboxylate

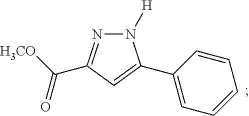

xi) methyl 5-(4-methylphenyl)-1H-pyrazole-3-carboxylate

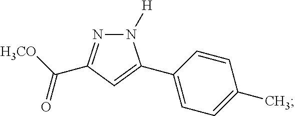

xii) methyl 5-(4-nitrophenyl)-1H-pyrazole-3-carboxylate

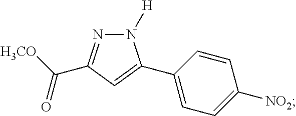

xiii) 2-[cyclohexyl(methyl)amino]-2-oxoethyl 5-(4-bromophenyl)-1H-pyrazole-3-carboxylate

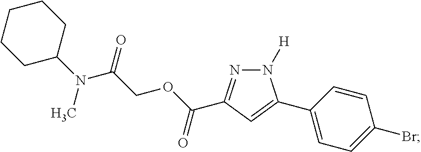

xiv) 3,3-dimethyl-2-oxobutyl 5-(4-bromophenyl)-1H-pyrazole-3-carboxylate

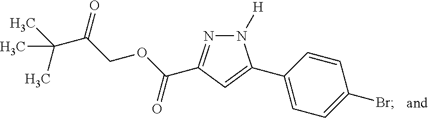

xv) 1-(tert-butylamino)-1-oxopropan-2-yl 5-(4-bromophenyl)-1H-pyrazole-3-carboxylate

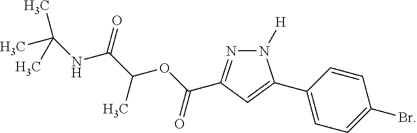

Table A provides non-limiting examples of Intestinal Alkaline Phosphatase activators and inhibitors according to this category.

TABLE A
IAP modulators (A)

| | | IC₅₀ | |
|---|---|---|---|
| No. | Compound | (μM) | n* |
| A1 | 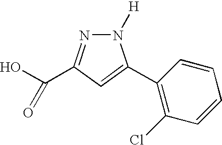 | 43.35 | 0.9625 |
| | 5-(2-chlorophenyl)-1H-pyrazole-3-carboxylic | | |
| | acid | | |
| A2 | 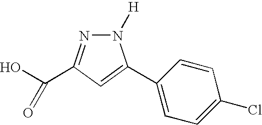 | >100 | — |
| | 5-(4-chlorophenyl)-1H-pyrazole-3-carboxylic | | |
| | acid | | |
| A3 | 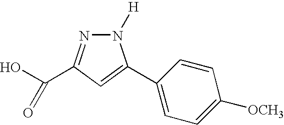 | >100 | — |
| | 5-(4-methoxyphenyl)-1H-pyrazole-3-carboxylic | | |
| | acid | | |
| A4 | 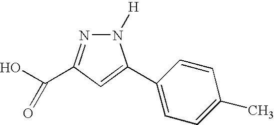 | >100 | — |
| | 5-(4-methylphenyl)-1H-pyrazole-3-carboxylic | | |
| | acid | | |
| A5 | 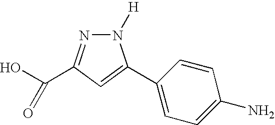 | >100 | — |
| | 5-(4-aminophenyl)-1H-pyrazole-3-carboxylic | | |
| | acid | | |
| A6 | 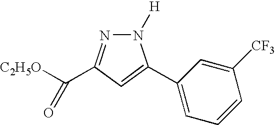 | 45.4 | −1.45 |
| | ethyl 5-[3-(trifluoromethyl)phenyl]-1H- | | |
| | pyrazole-3-carboxylate | | |
| A7 | 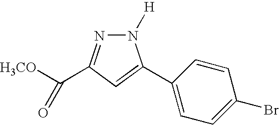 | >100 | — |
| | methyl 5-(4-bromophenyl)-1H-pyrazole-3- | | |
| | carboxylate | | |
| A8 | 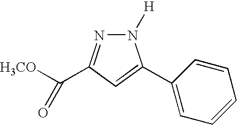 | >100 | — |
| | methyl 5-phenyl-1H-pyrazole-3-carboxylate | | |
| A9 | 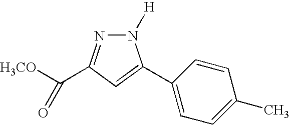 | >100 | — |
| | methyl 5-(4-methylphenyl)-1H-pyrazole-3- | | |
| | carboxylate | | |
| A10 | 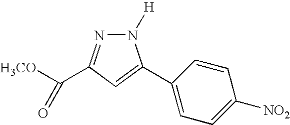 | >100 | — |
| | methyl 5-(4-nitrophenyl)-1H-pyrazole-3- | | |
| | carboxylate | | |
| A11 | 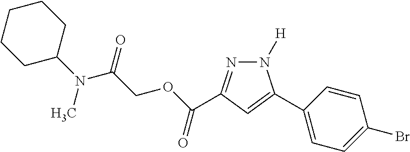 | >100 | — |
| | 2-[cyclohexyl(methyl)amino]-2-oxoethyl 5-(4- | | |
| | bromophenyl)-1H-pyrazole-3-carboxylate | | |
| A12 | 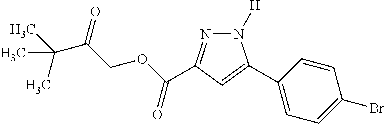 | 42.8 | −1.09 |
| | 3,3-dimethyl-2-oxobutyl 5-(4-bromophenyl)-1H- | | |
| | pyrazole-3-carboxylate | | |
| A13 | 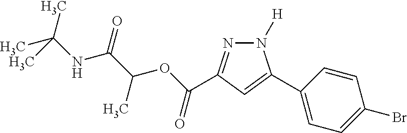 | 93.4 | −1 |
| | 1-(tert-butylamino)-l-oxopropan-2-yl 5-(4- | | |
| | bromophenyl)-1H-pyrazole-3-carboxylate | | |

*n represents the Hill coefficient. This coefficient is derived from the Hill equation which has the formula:

$$Θ = \frac{{[ L ]}^{n}}{{( {K}_{a} )}^{n} + {[ L ]}^{n}}$$

wherein Θ is the fraction of ligand binding sites filled, L is the inhibitor concentration, Kₐ is the inhibitor concentration producing half occupation of the ligand binding sites, and n is the Hill coefficient. Throughout Tables B-H the Hill coefficient, n, is the same as defined herein. Preferred activators have a Hill coefficient that is a negative number, for example, −0.023, −4, and −23.9. Preferred inhibitors have a Hill coefficient that is a positive number, for example, 0.01, 2.4, and 7.

Another embodiment of this aspect relates to modulators having the formula:

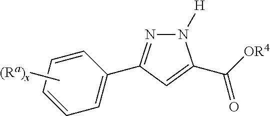

wherein R⁴ and R^{a} are the same as defined herein above.

Non-limiting examples of R units according to this embodiment includes units chosen from:
i) —CO₂H;
ii) —CO₂CH₃;
iii) —CO₂CHCH₃;
iv) —CO₂CF₃;
v) —CONHCH₃; and
vi) −CON(CH₃)₂.

Non-limiting examples of R¹ units according to this embodiment include the following:

Halogen substituted phenyl, for example, 2-fluorophenyl, 3-fluorophenyl, 4-fluorophenyl, 2,3-difluorophenyl, 2,4-difluorophenyl, 2,5-difluorophenyl, 2,6-difluorophenyl, 3,4-difluorophenyl, 3,5-difluorophenyl, 2-chlorophenyl, 3-chlorophenyl, 4-chlorophenyl, 2,3-dichlorophenyl, 2,4-dichlorophenyl, 2,5-dichlorophenyl, 2,6-dichlorophenyl, 3,4-dichlorophenyl, 3,5-dichlorophenyl, 2-bromophenyl, 3-bromophenyl, 4-bromophenyl, 2,3-dibromophenyl, 2,4-dibromophenyl, 2,5-dibromophenyl, 2,6-dibromophenyl, 3,4-dibromophenyl, and 3,5-dibromophenyl.

Alkyl substituted phenyl, for example, 2-methylphenyl, 3-methylphenyl, 4-methylphenyl, 2,3-dimethylphenyl, 2,4-dimethylphenyl, 2,5-dimethylphenyl, 2,6-dimethylphenyl, 3,4-dimethylphenyl, 3,5-dimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 3,5-diethylphenyl, 2-n-propylphenyl, 3-n-propylphenyl, 4-n-propylphenyl, 2,3-di-n-propylphenyl, 2,4-di-n-propylphenyl, 2,5-di-n-propylphenyl, 2,6-di-n-propylphenyl, 3,4-di-n-propylphenyl, 3,5-di-n-propylphenyl, 2-iso-propylphenyl, 3-iso-propylphenyl, 4-iso-propylphenyl, 2,3-di-iso-propylphenyl, 2,4-dii-so-propylphenyl, 2,5-di-iso-propylphenyl, 2,6-di-iso-propylphenyl, 3,4-di-iso-propylphenyl, and 3,5-di-iso-propylphenyl.

Alkoxy substituted phenyl, for example, 2-methoxyphenyl, 3-methoxyphenyl, 4-methoxyphenyl, 2,3-dimethoxyphenyl, 2,4-dimethoxyphenyl, 2,5-dimethoxyphenyl, 2,6-dimethoxyphenyl, 3,4-dimethoxyphenyl, 3,5-dimethoxyphenyl, 2-ethoxyphenyl, 3-ethoxyphenyl, 4-ethoxyphenyl, 2,3-diethoxyphenyl, 2,4-diethoxyphenyl, 2,5-diethoxyphenyl, 2,6-diethoxyphenyl, 3,4-diethoxyphenyl, 3,5-diethoxyphenyl, 2-propoxyphenyl, 3-propoxyphenyl, 4-propoxyphenyl, 2,3-dipropoxyphenyl, 2,4-dipropoxyphenyl, 2,5-dipropoxyphenyl, 2,6-dipropoxyphenyl, 3,4-dipropoxyphenyl, and 3,5-dipropoxyphenyl.

Hydroxy, nitro, cyano, thiol, and amino substituted phenyl, for example, 2-hydroxyphenyl, 3-hydroxyphenyl, 4-hydroxyphenyl, 2,3-dihydroxyphenyl, 2,4-dihydroxyphenyl, 2,5-dihydroxyphenyl, 2,6-dihydroxyphenyl, 3,4-dihydroxyphenyl, 3,5-dihydroxyphenyl, 2-nitrophenyl, 3-nitrophenyl, 4-nitrophenyl, 2,3-dinitrophenyl, 2,4-dinitrophenyl, 2,5-dinitrophenyl, 2,6-dinitrophenyl, 3,4-dinitrophenyl, 3,5-dinitrophenyl, 2-cyanophenyl, 3-cyanophenyl, 4-cyanophenyl, 2,3-dicyanophenyl, 2,4-dicyanophenyl, 2,5-dicyanophenyl, 2,6-dicyanophenyl, 3,4-dicyanophenyl, 3,5-dicyanophenyl, 2-thiophenyl, 3-thiophenyl, 4-thiophenyl, 2,3-dithiophenyl, 2,4-dithiophenyl, 2,5-dithiophenyl, 2,6-dithiophenyl, 3,4-dithiophenyl, 3,5-dithiophenyl, 2-aminophenyl, 3-aminophenyl, 4-aminophenyl, 2,3-diaminophenyl, 2,4-diaminophenyl, 2,5-diaminophenyl, 2,6-diaminophenyl, 3,4-diaminophenyl, and 3,5-diaminophenyl.

Trifluoromethyl and sulfoxy substituted phenyl, for example, 2-trifluoromethylphenyl, 3-trifluoromethylphenyl, 4-trifluoromethylphenyl, 2,3-ditrifluoromethylphenyl, 2,4-ditrifluoromethylphenyl, 2,5-ditrifluoromethylphenyl, 2,6-ditrifluoromethylphenyl, 3,4-ditrifluoromethylphenyl, 3,5-ditrifluoromethylphenyl, 2-sulfoxyphenyl, 3-sulfoxyphenyl, 4-sulfoxyphenyl, 2,3-disulfoxyphenyl, 2,4-disulfoxyphenyl, 2,5-disulfoxyphenyl, 2,6-disulfoxyphenyl, 3,4-disulfoxyphenyl, and 3,5-disulfoxyphenyl.

One iteration of this embodiment relates to compounds having the formula:

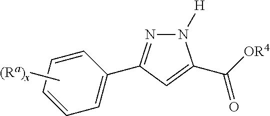

wherein R⁴ is chosen from:
i) hydrogen;
ii) C₁-C₄ linear or branched alkyl; or
iii) —[CH₂]_{w}C(O)N(R^{8a})(R^{8b}); and
   each R^{a} is chosen from:
i) C₁-C₄ linear or branched alkyl;
ii) C₁-C₄ linear or branched alkoxy;
iii) —OH;
iv) —F;
v) —Cl;
vi) —Br;
vii) —NO₂;
viii) —NH₂;
ix) —CF₃; and
x) two adjacent R^{a} units can be taken together to form a fused ring wherein R comprises from 8 to 12 atoms;
   the index x is an integer from 0 to 5, and the integer w is from 0 to 2.

Non-limiting examples of this embodiment include:
i) 3-(4-hydroxyphenyl)-1H-pyrazole-5-carboxylic acid

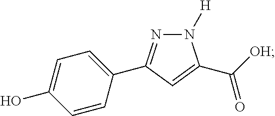

ii) 3-(2-hydroxyphenyl)-1H-pyrazole-5-carboxylic acid

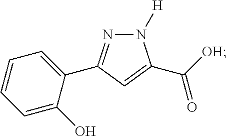

iii) 3-(4-isopropylphenyl)-1H-pyrazole-5-carboxylic acid

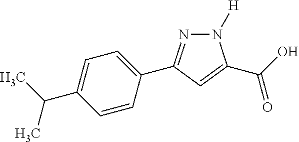

iv) 3-(4-fluorophenyl)-1H-pyrazole-5-carboxylic acid

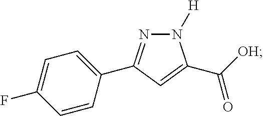

v) 3-(3-methoxyphenyl)-1H-pyrazole-5-carboxylic acid

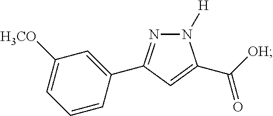

vi) 3-(4-ethylphenyl)-1H-pyrazole-5-carboxylic acid

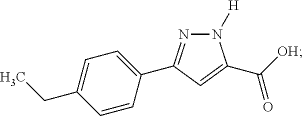

vii) 3-(2,4-dimethylphenyl)-1H-pyrazole-5-carboxylic acid

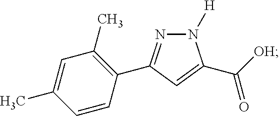

viii) 3-(3,4-dimethylphenyl)-1H-pyrazole-5-carboxylic acid

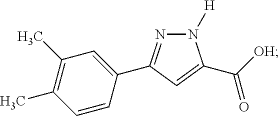

ix) 3-(4-ethoxyphenyl)-1H-pyrazole-5-carboxylic acid

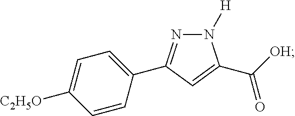

x) 3-(2,4-diethoxyphenyl)-1H-pyrazole-5-carboxylic acid

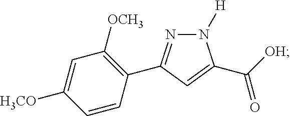

xi) 3-(2,3-dihydrobenzo[b][1,4]dioxyin-6-yl)-1H-pyrazole-5-carboxylic acid

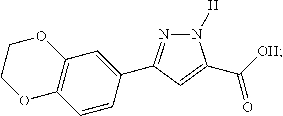

xii) methyl 3-(2,4-dichlorophenyl)-1H-pyrazole-5-carboxylate

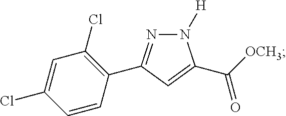

xiii) methyl 3-(2,4-dimethylphenyl)-1H-pyrazole-5-carboxylate

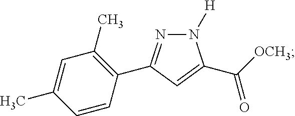

xiv) methyl 3-(4-methoxyphenyl)-1H-pyrazole-5-carboxylate

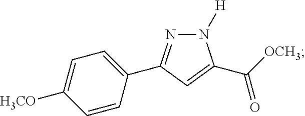

xv) methyl 3-(4-butoxyphenyl)-1H-pyrazole-5-carboxylate

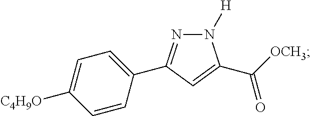

xvi) ethyl 3-(2,3-dihydrobenzo[b][1,4]dioxyin-6-yl)-1H-pyrazole-5-carboxylate

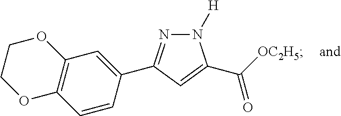

xvii) ethyl 3-(4-chlorophenyl)-1H-pyrazole-5-carboxylate

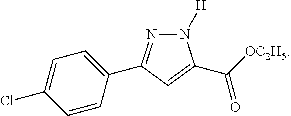

Table B provides non-limiting examples of Intestinal Alkaline Phosphatase activators and inhibitors according to this category.

TABLE B
IAP modulators (B)

| | | IC₅₀ | |
|---|---|---|---|
| No. | Compound | (μM) | n* |
| B1 | 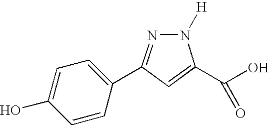 | 79.2 | 1.1 |
| | 3-(4-hydroxyphenyl)-1H-pyrazole-5-carboxylic acid | | |
| B2 | 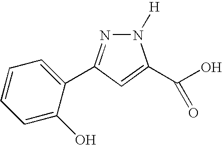 | 7.85 | −1.92 |
| | 3-(2-hydroxyphenyl)-1H-pyrazole-5-carboxylic acid | | |
| B3 | 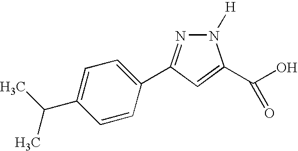 | 98.3 | −4.38 |
| | 3-(4-isopropylphenyl)-1H-pyrazole-5-carboxylic | | |
| | acid | | |
| B4 | 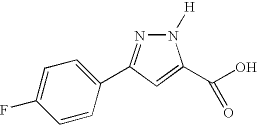 | >100 | — |
| | 3-(4-fluorophenyl)-1H-pyrazole-5-carboxylic | | |
| | acid | | |
| B5 | 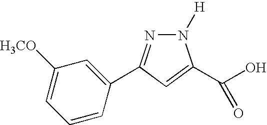 | >100 | — |
| | 3-(3-methoxyphenyl)-1H-pyrazole-5-carboxylic | | |
| | acid | | |
| B6 | 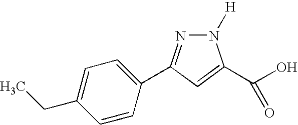 | >100 | — |
| | 3-(4-ethylphenyl)-1H-pyrazole-5-carboxylic acid | | |
| B7 | 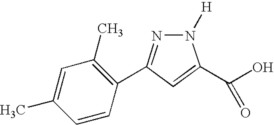 | >100 | — |
| | 3-(2,4-dimethylphenyl)-1H-pyrazole-5-carboxylic | | |
| | acid | | |
| B8 | 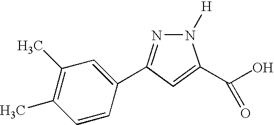 | >100 | — |
| | 3-(3,4-dimethylphenyl)-1H-pyrazole-5- | | |
| | carboxylic acid | | |
| B9 | 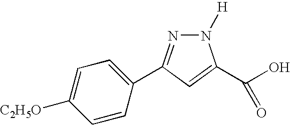 | >100 | — |
| | 3-(4-ethoxyphenyl)-1H-pyrazole-5-carboxylic | | |
| | acid | | |
| B10 | 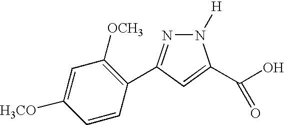 | >100 | — |
| | 3-(2,4-diethoxyphenyl)-1H-pyrazole-5- | | |
| | carboxylic acid | | |
| B11 | 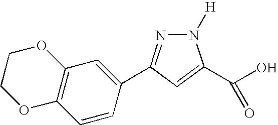 | >100 | — |
| B12 | 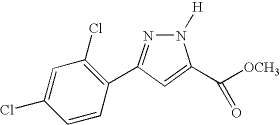 | 9.32 | −1.3 |
| | methyl 3-(2,4-dichlorophenyl)-1H-pyrazole-5- | | |
| | carboxylate | | |
| B13 | 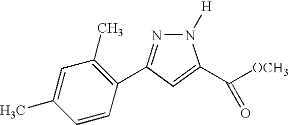 | 66.1 | −3.035 |
| | methyl 3-(2,4-dimethylphenyl)-1H-pyrazole-5- | | |
| | carboxylate | | |
| B14 | 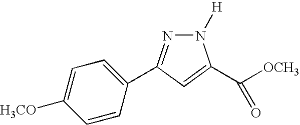 | >100 | — |
| | methyl 3-(4-methoxyphenyl)-1H-pyrazole-5- | | |
| | carboxylate | | |
| B15 | 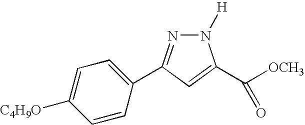 | >100 | — |
| | methyl 3-(4-butoxyphenyl)-1H-pyrazole-5- | | |
| | carboxylate | | |
| B16 | 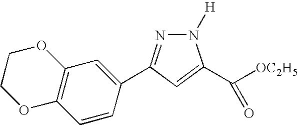 | >100 | — |
| | ethyl 3-(2,3-dihydrobenzo[b][1,4]dioxyin-6-yl)- | | |
| | 1H-pyrazole-5-carboxylate | | |
| B17 | 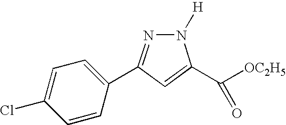 | >100 | — |
| | ethyl 3-(4-chlorophenyl)-1H-pyrazole-5- | | |
| | carboxylate | | |

A further aspect of this category relates to Intestinal Alkaline Phosphatase modulators having the formula:

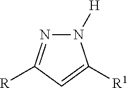

wherein R is a unit having the formula —C(O)R⁴ and R¹ is substituted or unsubstituted C₁₀ aryl (naphthalenyl) or R¹ is a unit having the formula —C(O)R⁴ and R is substituted or unsubstituted C₁₀ aryl (naphthalenyl). One embodiment of this aspect relates to modulators having the formula:

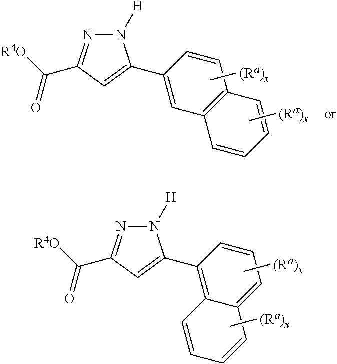

wherein each R^{a} is the same as defined herein above, the index x is from 0 to 4. R⁴ is chosen from:
a) hydrogen;
b) substituted or unsubstituted C₁-C₁₀ linear, branched, or cyclic alkyl;
c) —OR⁵ wherein R⁵ is chosen from:
i) hydrogen;
ii) substituted or unsubstituted C₁-C₄ linear or branched alkyl; wherein each substitution on the alkyl chain is independently chosen from:
a) halogen; and
b) —[C(R^{7a})(R^{7b})]_{w}C(O)R⁶;
   R⁶ is hydroxy, C₁-C₄ linear or branched alkoxy, or —N(R^{8a})(R^{8b}), each R^{8a} and R^{8b} is independently chosen from hydrogen or C₁-C₁₀ linear, branched or cyclic alkyl;
c) —[C(R^{7a})(R^{7b})]_{w}N(R^{9a}) (R^{9b});
   each R^{9a} and R^{9b} is independently chosen from hydrogen or C₁-C₁₀ linear, branched or cyclic alkyl; or R^{9a} and R^{9b} can be taken together to form a ring having from 3 to 7 atoms; each R^{7a} and R^{7b} is independently hydrogen or C₁-C₄ linear or branched alkyl; the index w is an integer from 0 to 5.

Another embodiment of this aspect relates to modulators having the formula:

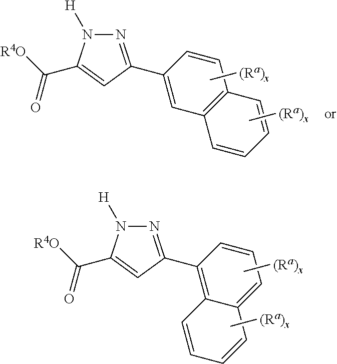

wherein each R^{a} is the same as defined herein above, the index x is from 0 to 4. R⁴ is chosen from:
a) hydrogen;
b) substituted or unsubstituted C₁-C₁₀ linear, branched, or cyclic alkyl;
c) —OR⁵ wherein R⁵ is chosen from:
i) hydrogen;
ii) substituted or unsubstituted C₁-C₄ linear or branched alkyl; wherein each substitution on the alkyl chain is independently chosen from:
a) halogen; and
b) —[C(R^{7a})(R^{7b})]_{w}C(O)R⁶;
   R⁶ is hydroxy, C₁-C₄ linear or branched alkoxy, or —N(R^{8a})(R^{8b}), each R^{8a} and R^{8b} is independently chosen from hydrogen or C₁-C₁₀ linear, branched or cyclic alkyl;
c) —[C(R^{7a})(R^{7b})]_{w}N(R^{9a})(R^{9b});
   each R^{9a} and R^{9b} is independently chosen from hydrogen or C₁-C₁₀ linear, branched or cyclic alkyl; or R^{9a} and R^{9b} can be taken together to form a ring having from 3 to 7 atoms; each R^{7a} and R^{7b} is independently hydrogen or C₁-C₄ linear or branched alkyl; the index w is an integer from 0 to 5.

Table C provides non-limiting examples of Intestinal Alkaline Phosphatase activators and inhibitors according to this category.

TABLE C
IAP modulator (C)

| | | IC₅₀ | |
|---|---|---|---|
| No. | Compound | (μM) | n* |
| C1 | 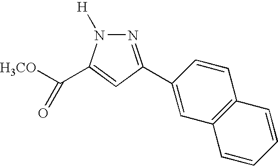 | >100 | — |
| | methyl 3-(nahthylen-2-yl)-1H-pyrazole-5-carboxylate | | |

A further aspect of this category relates to Intestinal Alkaline Phosphatase modulators having the formula:

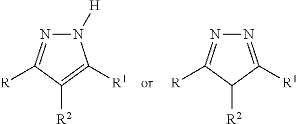

wherein R is a unit having the formula —C(O)R⁴ and R¹ is substituted or unsubstituted C₆ aryl (phenyl) or R¹ is a unit having the formula —C(O)R⁴ and R is substituted or unsubstituted C₆ aryl (phenyl), R² is methyl, and R, R¹, and R⁴ are the same as defined herein above.

A non-limiting example of modulators according to this aspect includes 4-methyl-5-phenyl-1H-pyrazole-3-carboxylic acid having the formula:

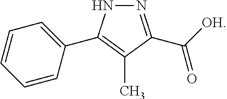

A yet further aspect of this category relates to Intestinal Alkaline Phosphatase modulators having the formula:

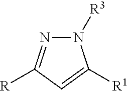

wherein R is a unit having the formula —C(O)R⁴ and R¹ is substituted or unsubstituted C₆ aryl (phenyl) or R¹ is a unit having the formula —C(O)R⁴ and R is substituted or unsubstituted C₆ aryl (phenyl), R³ is methyl, and R, R¹, and R⁴ are the same as defined herein above.

Non-limiting examples of modulators according to this aspect include:
i) 3-(4-fluorophenyl)-1-methyl 1H-pyrazole-5 carboxylic acid:

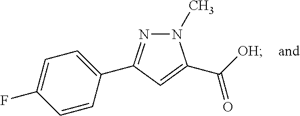

ii) 5-(4-fluorophenyl)-1-methyl 1H-pyrazole-3 carboxylic acid:

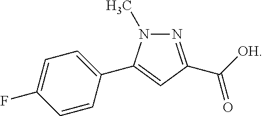

Table D provides non-limiting examples of Intestinal Alkaline Phosphatase activators and inhibitors according to this category.

TABLE D
IAP modulators (D)

| | | IC₅₀ | |
|---|---|---|---|
| No. | Compound | (μM) | n* |
| D1 | 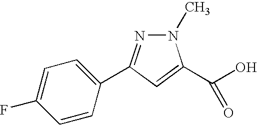 | >100 | — |
| | 3-(4-fluorophenyl)-1-methyl 1H-pyrazole-5 carboxylic | | |
| | acid | | |
| D2 | 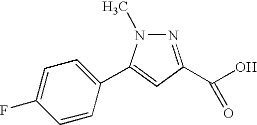 | >100 | — |
| | 5-(4-fluorophenyl)-1-methyl 1H-pyrazole-3 carboxylic | | |
| | acid | | |
| D3 | 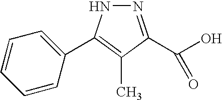 | >100 | — |
| | 4-methyl-5-phenyl-1H-pyrazole-3-carboxylic acid | | |

Another aspect of this category relates to Intestinal Alkaline Phosphatase modulators having the formula:

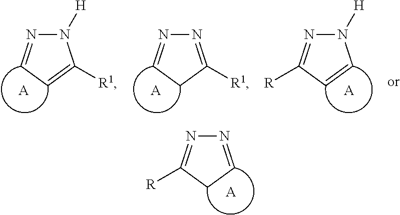

wherein A is one or more substituted or unsubstituted cycloalkyl, aryl, heterocyclic, or heteroaryl rings having from 3 to 14 carbon atoms and from 1 to 5 heteroatoms chosen from oxygen, nitrogen, sulfur, or combinations thereof.

A first embodiment of this aspect relates to fused rings having the formula:

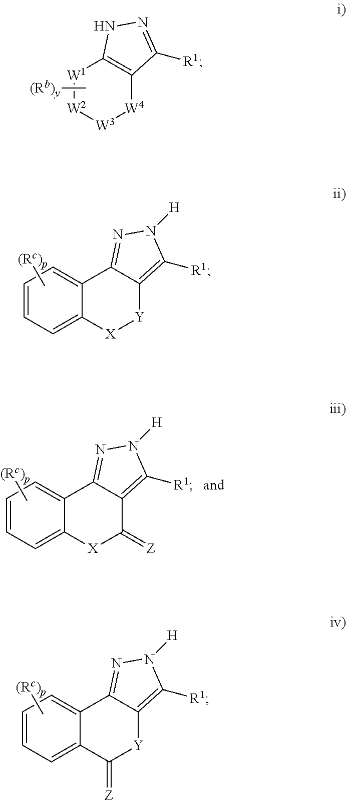

wherein W¹, W², W³, W⁴, X, and Y are each independently chosen from:
ii) —CH═;
iii) —CH₂—;
iv) —N═;
v) —NH—;
vi) —S—; and
vii) —O—;
   wherein the hydrogen atoms of W¹, W², W³, W⁴, X, and Y can be substituted by a R^{c} unit; Z is O, S, or NH.

Each R^{b} represents from 1 to 5 optionally present substitutions for a hydrogen atom on a ring, as such the index y is an integer from 0 to 5. Each R^{a} is independently chosen from
i) C₁-C₁₂ substituted or unsubstituted linear, branched, or cyclic alkyl;
ii) C₂-C₁₂ substituted or unsubstituted linear, branched, or cyclic alkenyl;
iii) C₂-C₁₂ substituted or unsubstituted linear or branched alkynyl;
iv) C₆ or C₁₀ substituted or unsubstituted aryl;
v) C₁-C₉ substituted or unsubstituted heterocyclic;
vi) C₁-C₁₁ substituted or unsubstituted heteroaryl;
vii) —[C(R^{39a})(R^{39b})]ₘOR²⁵;
   R²⁵ is chosen from:
   a) —H;
   b) C₁-C₁₂ substituted or unsubstituted linear, branched, or cyclic alkyl;
   c) C₆ or C₁₀ substituted or unsubstituted aryl or alkylenearyl;
   d) C₁-C₉ substituted or unsubstituted heterocyclic;
   e) C₁-C₁₁ substituted or unsubstituted heteroaryl;
viii) —[C(R^{39a})(R^{39b})]ₘN(R^{26a})(R^{26b});
   R^{26a} and R^{26b} are each independently chosen from:
   a) —H;
   b) —OR²⁷;
   R²⁷ is hydrogen or C₁-C4 linear alkyl;
   c) C₁-C₁₂ substituted or unsubstituted linear, branched, or cyclic alkyl;
   d) C₆ or C₁₀ substituted or unsubstituted aryl;
   e) C₁-C₉ substituted or unsubstituted heterocyclic;
   f) C₁-C₁₁ substituted or unsubstituted heteroaryl; or
   g) R^{26a} and R^{26b} can be taken together to form a substituted or unsubstituted ring having from 3 to 10 carbon atoms and from 0 to 3 heteroatoms chosen from oxygen, nitrogen, and sulfur;
ix) —[C(R^{39a})(R^{39b})]ₘC(O)R²⁸;
   R²⁸ is
   a) C₁-C₁₂ substituted or unsubstituted linear, branched, or cyclic alkyl;
   b) —OR²⁹;
   R²⁹ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl;
   c) —N(R^{30a})(R^{30b});
   R^{30a} and R^{30b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{30a} and R^{30b} can be taken together to form a substituted or unsubstituted ring having from 3 to 10 carbon atoms and from 0 to 3 heteroatoms chosen from oxygen, nitrogen, and sulfur;
x) —[C(R^{39a})(R^{39b})]ₘOC(O)R³¹;
   R³¹ is
   a) C₁-C₁₂ substituted or unsubstituted linear, branched, or cyclic alkyl;
   b) —N(R^{32a})(R^{32b});
   R^{32a} and R^{32b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{32a} and R^{32b} can be taken together to form a substituted or unsubstituted ring having from 3 to 10 carbon atoms and from 0 to 3 heteroatoms chosen from oxygen, nitrogen, and sulfur;
xi) —[C(R^{39a})(R^{39b})]ₘNR³³C(O)R³⁴;
   R³³ is:
   a) —H; or
   b) C₁-C₄ substituted or unsubstituted linear, branched, or cyclic alkyl;
   R³⁴ is
   a) C₁-C₁₂ substituted or unsubstituted linear, branched, or cyclic alkyl;
   b) —N(R^{35a})(R^{35b});
   R^{35a} and R^{35b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{35a} and R^{35b} can be taken together to form a substituted or unsubstituted ring having from 3 to 10 carbon atoms and from 0 to 3 heteroatoms chosen from oxygen, nitrogen, and sulfur;
xii) —[C(R^{39a})(R^{39b})]ₘCN;
xiii) —[C(R^{39a})(R^{39b})]ₘNO₂;
xiv) —[C(R^{39a})(R^{39b})]ₘR³⁶;
   R³⁶ is C₁-C₁₀ linear, branched, or cyclic alkyl substituted by from 1 to 21 halogen atoms chosen from —F, —Cl, —Br, or —I;
xv) —[C(R^{39a})(R^{39b})]ₘSO₂R³⁷;
   R³⁷ is hydrogen, hydroxyl, substituted or unsubstituted C₁-C₄ linear or branched alkyl; substituted or unsubstituted C₆, C₁₀, or C₁₄ aryl; C₇-C₁₅ alkylenearyl; C₁-C₉ substituted or unsubstituted heterocyclic; or C₁-C₁₁ substituted or unsubstituted heteroaryl;
iii) two R units on the same carbon atom can be taken together to form a unit chosen from ═O, ═S, or ═NR³⁸;
   R³⁸ is hydrogen, hydroxyl, C₁-C₄ linear or branched alkyl, or C₁-C₄ linear or branched alkoxy;
      R^{39a} and R^{39b} are each independently hydrogen or C₁-C₄ alkyl; and
      the index y is an integer from 0 to 5.

Each R^{c} represents from 1 to 5 optionally present substitutions for a hydrogen atom on a ring, as such the index p is an integer from 0 to 5. Each R^{c} is independently chosen from
i) C₁-C₁₂ substituted or unsubstituted linear, branched, or cyclic alkyl;
ii) C₂-C₁₂ substituted or unsubstituted linear, branched, or cyclic alkenyl;
iii) C₂-C₁₂ substituted or unsubstituted linear or branched alkynyl;
iv) C₆ or C₁₀ substituted or unsubstituted aryl;
v) C₁-C₉ substituted or unsubstituted heterocyclic;
vi) C₁-C₁₁ substituted or unsubstituted heteroaryl;
vii) —[C(R^{54a})(R^{54b})]_{q}OR⁴⁰;
   R⁴⁰ is chosen from:
   a) —H;
   b) C₁-C₁₂ substituted or unsubstituted linear, branched, or cyclic alkyl;
   c) C₆ or C₁₀ substituted or unsubstituted aryl or alkylenearyl;
   d) C₁-C₉ substituted or unsubstituted heterocyclic;
   e) C₁-C₁₁ substituted or unsubstituted heteroaryl;
viii) —[C(R^{54a})(R^{54b})]_{q}N(R^{41a})(R^{41b});
   R^{41a} and R^{41b} are each independently chosen from:
   a) —H;
   b) —OR⁴²;
   R⁴² is hydrogen or C₁-C₄ linear alkyl;
   c) C₁-C₁₂ substituted or unsubstituted linear, branched, or cyclic alkyl;
   d) C₆ or C₁₀ substituted or unsubstituted aryl;
   e) C₁-C₉ substituted or unsubstituted heterocyclic;
   f) C₁-C₁₁ substituted or unsubstituted heteroaryl; or
   g) R^{41a} and R^{41b} can be taken together to form a substituted or unsubstituted ring having from 3 to 10 carbon atoms and from 0 to 3 heteroatoms chosen from oxygen, nitrogen, and sulfur;
ix) —[C(R^{54a})(R^{54b})]_{q}C(O)R⁴³;
   R⁴³ is
   a) C₁-C₁₂ substituted or unsubstituted linear, branched, or cyclic alkyl;
   b) —OR⁴⁴;
   R⁴⁴ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl;
   c) —N(R^{45a})(R^{45b});
   R^{45a} and R^{45b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{45a} and R^{45b} can be taken together to form a substituted or unsubstituted ring having from 3 to 10 carbon atoms and from 0 to 3 heteroatoms chosen from oxygen, nitrogen, and sulfur;
x) —[C(R^{54a})(R^{54b})]_{q}OC(O)R⁴⁶;
   R⁴⁶ is
   a) C₁-C₁₂ substituted or unsubstituted linear, branched, or cyclic alkyl;
   b) —N(R^{47a})(R^{47b});
   R^{47a} and R^{47b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{47a} and R^{47b} can be taken together to form a substituted or unsubstituted ring having from 3 to 10 carbon atoms and from 0 to 3 heteroatoms chosen from oxygen, nitrogen, and sulfur;
xi) —[C(R^{54a})(R^{54b})]_{q}NR⁴⁸C(O)R⁴⁹;
   R⁴⁸ is:
   a) —H; or
   b) C₁-C₄ substituted or unsubstituted linear, branched, or cyclic alkyl;
   R⁴⁹ is
   a) C₁-C₁₂ substituted or unsubstituted linear, branched, or cyclic alkyl;
   b) —N(R^{50a})(R^{50b});
   R^{50a} and R^{50b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{50a} and R^{50b} can be taken together to form a substituted or unsubstituted ring having from 3 to 10 carbon atoms and from 0 to 3 heteroatoms chosen from oxygen, nitrogen, and sulfur;
xii) —[C(R^{54a})(R^{54b})]_{q}CN;
xiii) —[C(R^{54a})(R^{54b})]_{q}NO₂;
xiv) —[C(R^{54a})(R^{54b})]_{q}R⁵¹;
   R⁵¹ is C₁-C₁₀ linear, branched, or cyclic alkyl substituted by from 1 to 21 halogen atoms chosen from —F, —Cl, —Br, or —I;
xv) —[C(R^{54a})(R^{54b})]_{q}SO₂R⁵²;
   R⁵² is hydrogen, hydroxyl, substituted or unsubstituted C₁-C₄ linear or branched alkyl; substituted or unsubstituted C₆, C₁₀, or C₁₋₄ aryl; C₇-C₁₅ alkylenearyl; C₁-C₉ substituted or unsubstituted heterocyclic; or C₁-C₁₁ substituted or unsubstituted heteroaryl;
iv) two R^{c} units on the same carbon atom can be taken together to form a unit chosen from ═O, ═S, or ═NR⁵³;
   R⁵³ is hydrogen, hydroxyl, C₁-C₄ linear or branched alkyl, or C₁-C₄ linear or branched alkoxy;
      R^{54a} and R^{54b} are each independently hydrogen or C₁-C₄ alkyl; and
      the index p is an integer from 0 to 5.

The R^{b} and R^{c} units disclosed herein can be further substituted by one or more organic radicals independently chosen from:
i) C₁-C₁₂ linear, branched, or cyclic alkyl, alkenyl, and alkynyl;
ii) substituted or unsubstituted C₆ or C₁₀ aryl;
iii) substituted or unsubstituted C₆ or C₁₀ alkylenearyl;
iv) substituted or unsubstituted C₁-C₉ heterocyclic rings;
v) substituted or unsubstituted C₁-C₉ heteroaryl rings;
vi) —(CR^{102a}R^{102b})_{z}OR¹⁰¹;
vii) —(CR^{102a}R^{102b})_{z}C(O)R¹⁰¹;
viii) —(CR^{102a}R^{102b})_{z}C(O)OR¹⁰¹;
iii) —(CR^{102a}R^{102b})_{z}C(O)N(R¹⁰¹)₂;
ix) —(CR^{102a}R^{102b})_{z}N(R¹⁰¹)₂;
xi) halogen;
xii) —(CR^{102a}R^{102b})_{z}CN;
xiii) —(CR^{102a}R^{102b})_{z}NO₂;
xiv) —CHⱼXₖ; wherein X is halogen, the index j is an integer from 0 to 2, j+k=3;
XV) —(CR^{102a}R^{102b})_{z}SR¹⁰¹;
xvi) —(CR^{102a}R^{102b})_{z}SO₂R¹⁰¹; and
xvii) —(CR^{102a}R^{102b})_{z}SO₃R¹⁰¹;
   wherein each R¹⁰¹ is independently hydrogen, substituted or unsubstituted C₁-C₄ linear, branched, or cyclic alkyl, phenyl, benzyl, heterocyclic, or heteroaryl; or two R¹⁰¹ units can be taken together to form a ring comprising 3-7 atoms; R^{102a} and R^{102b} are each independently hydrogen or C₁-C₄ linear or branched alkyl; the index z is from 0 to 4.

Non-limiting examples of this aspect are modulators having the formula:
i) 2,4-dihydrochromeno[3,4-c]pyrazole-3-carboxylic acid

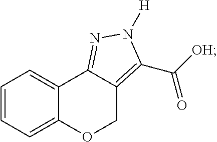

ii) (2,4-dihydrochromeno[3,4-c]pyrazol-3-yl)(pyrrolidin-1-yl)methanone

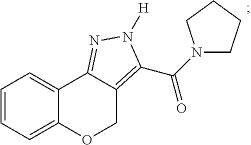

iii) ethyl 2,4-dihydrochromeno[3,4-c]pyrazole-3-carboxylate

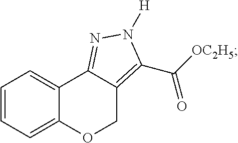

iv) 3-(4-methoxyphenyl)-4-methylpyrano[2,3-c]pyrazol-6(1H)-one

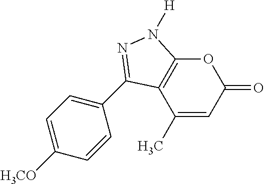

v) 3-(4-methoxyphenyl)-4-methylpyrano[2,3-c]prazol-6-ol

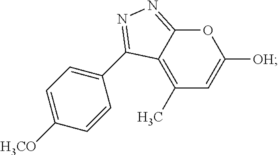

vi) 4-methyl-3-phenylpyrano[2,3-c]pyrazol-6-ol

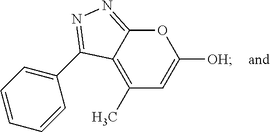

vii) 4-(2-hydroxyethyl)-3-phenylpyrano[2,3-c]pyrazol-6(1H)-one

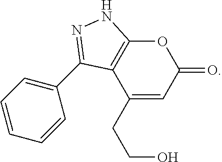

Table E provides non-limiting examples of Intestinal Alkaline Phosphatase activators and inhibitors according to this category.

TABLE E
IAP modulators (E)

| | | IC₅₀ | |
|---|---|---|---|
| No. | Compound | (μM) | n* |
| E1 | 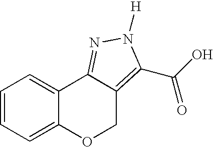 | 13.9 | 0.533 |
| | 2,4-dihydrochromeno[3,4-c]pyrazole-3- | | |
| | carboxylic acid | | |
| E2 | 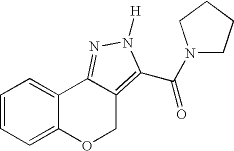 | >100 | — |
| | (2,4-dihydrochromeno[3,4-c]pyrazol-3- | | |
| | yl)(pyrrolidin-1-yl)methanone | | |
| E3 | 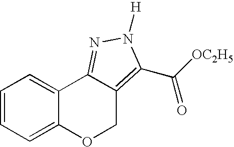 | >100 | — |
| | ethyl 2,4-dihydrochromeno[3,4-c]pyrazole-3- | | |
| | carboxylate | | |
| E4 | 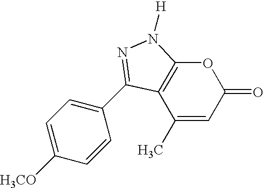 | 23.75 | −1.49 |
| | 3-(4-methoxyphenyl)-4-methylpyrano[2,3- | | |
| | c]pyrazol-6(1H)-one | | |
| E5 | 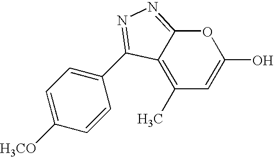 | 21.1 | −1.89 |
| | 3-(4-methoxyphenyl)-4-methylpyrano[2,3- | | |
| | c]prazol-6-ol | | |
| E6 | 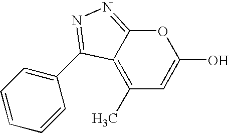 | 62.7 | −5 |
| | 4-methyl-3-phenylpyrano[2,3-c]pyrazol-6-ol | | |
| E7 | 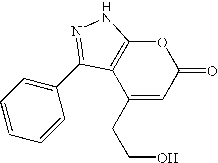 | >100 | — |
| | 4-(2-hydroxyethyl)-3-phenylpyrano[2,3- | | |
| | c]pyrazol-6(1H)-one | | |

Another category of Intestinal Adrenaline Phosphatase modulators has the formula:

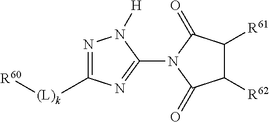

wherein R⁶⁰ is chosen from:
i) hydrogen;
ii) substituted or unsubstituted C₆ or C₁₀ aryl;
iii) substituted or unsubstituted C₁-C₉ heteroaryl; or
iv) substituted or unsubstituted C₁-C₉ heterocyclic;
   R⁶¹ and R⁶² are taken together to form a ring chosen from:
i) saturated or unsaturated cycloalkyl;
ii) saturated or unsaturated bicycloalkyl; or
iii) aryl;
   L is a linking unit having from 1 to 5 carbon atoms; and
   the index k is 0 or 1.

R⁶⁰ in one embodiment is hydrogen. The disclosed modulators according to this embodiment of R⁶⁰ have the formula:

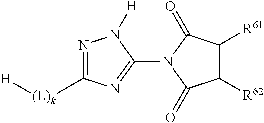

In another embodiment, R⁶⁰ is substituted or unsubstituted phenyl (C₆ aryl), substituted or unsubstituted naphthalene-1-yl (C₁₀ aryl), or substituted or unsubstituted naphthalene-2-yl (C₁₀ aryl). The disclosed modulators according to this embodiment of R⁶⁰ have the formula:

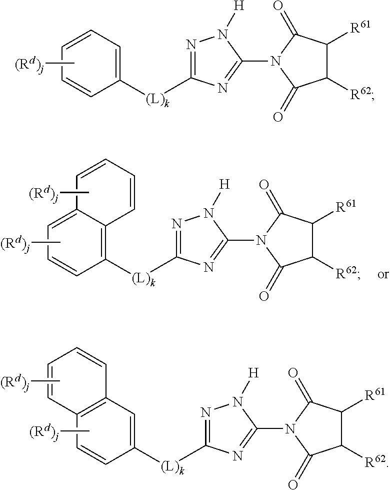

In a further embodiment, R⁶⁰ is substituted or unsubstituted C₁-C₉ heteroaryl, or substituted or unsubstituted C₁-C₉ heterocyclic. The disclosed modulators according to this embodiment of R⁶⁰ have the formula:

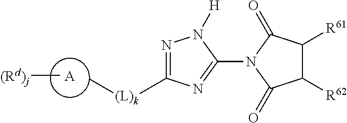

wherein A is a substituted or unsubstituted C₁-C₉ heteroaryl ring, or substituted or unsubstituted C₁-C₉ heterocyclic ring.

Each R^{d} represents from 1 to 5 optionally present substitutions for a hydrogen atom on a ring, as such the index j is an integer from 0 to 5. Each R^{d} is independently chosen from
i) C₁-C₁₂ substituted or unsubstituted linear, branched, or cyclic alkyl;
ii) C₂-C₁₂ substituted or unsubstituted linear, branched, or cyclic alkenyl;
iii) C₂-C₁₂ substituted or unsubstituted linear or branched alkynyl;
iv) C₆ or C₁₀ substituted or unsubstituted aryl;
v) C₁-C₉ substituted or unsubstituted heterocyclic;
vi) C₁-C₁₁ substituted or unsubstituted heteroaryl;
vii) —[C(R^{69a})(R^{69b})]ᵤOR⁵⁵;
   R⁵⁵ is chosen from:
   a) —H;
   b) C₁-C₁₂ substituted or unsubstituted linear, branched, or cyclic alkyl;
   c) C₆ or C₁₀ substituted or unsubstituted aryl or alkylenearyl;
   d) C₁-C₉ substituted or unsubstituted heterocyclic;
   e) C₁-C₁₁ substituted or unsubstituted heteroaryl;
viii) —[C(R^{69a})(R^{69b})]ᵤN(R^{56a})(R^{56b});
   R^{56a} and R^{56b} are each independently chosen from:
   a) —H;
   b) —OR⁵⁷
   R⁵⁷ is hydrogen or C₁-C₄ linear alkyl;
   c) C₁-C₁₂ substituted or unsubstituted linear, branched, or cyclic alkyl;
   d) C₆ or C₁₀ substituted or unsubstituted aryl;
   e) C₁-C₉ substituted or unsubstituted heterocyclic;
   f) C₁-C₁₁ substituted or unsubstituted heteroaryl; or
   g) R^{56a} and R^{56b} can be taken together to form a substituted or unsubstituted ring having from 3 to 10 carbon atoms and from 0 to 3 heteroatoms chosen from oxygen, nitrogen, and sulfur;
ix) —[C(R^{69a})(R^{69b})]ᵤC(O)R⁵⁸;
   R⁵⁸ is
   a) C₁-C₁₂ substituted or unsubstituted linear, branched, or cyclic alkyl;
   b) —OR⁵⁹;
   R⁵⁹ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl;
   c) —N(R^{60a})(R^{60b});
   R^{60a} and R^{60b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{60a} and R^{60b} can be taken together to form a substituted or unsubstituted ring having from 3 to 10 carbon atoms and from 0 to 3 heteroatoms chosen from oxygen, nitrogen, and sulfur;
x) —[C(R^{69a})(R^{69b})]ᵤOC(O)R⁶¹;
   R⁶¹ is:
   a) C₁-C₁₂ substituted or unsubstituted linear, branched, or cyclic alkyl;
   b) —N(R^{62a})(R^{62b});
   R^{62a} and R^{62b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{62a} and R^{62b} can be taken together to form a substituted or unsubstituted ring having from 3 to 10 carbon atoms and from 0 to 3 heteroatoms chosen from oxygen, nitrogen, and sulfur;
xi) —[C(R^{69a}) (R^{69b})]ᵤNR⁶³C(O)R⁶⁴;
   R⁶³ is:
   a) —H; or
   b) C₁-C₄ substituted or unsubstituted linear, branched, or cyclic alkyl;
   R⁶⁴ is:
   a) C₁-C₁₂ substituted or unsubstituted linear, branched, or cyclic alkyl;
   b) —N(R^{65a})(R^{65b});
   R^{65a} and R^{65b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{65a} and R^{65b} can be taken together to form a substituted or unsubstituted ring having from 3 to 10 carbon atoms and from 0 to 3 heteroatoms chosen from oxygen, nitrogen, and sulfur;
xii) —[C(R^{69a})(R^{69b})]ᵤCN;
xiii) —[C(R^{69a})(R^{69b})]ᵤNO₂;
xiv) —[C(R^{69a})(R^{69b})]ᵤR⁶⁶;
   R⁶⁶ is C₁-C₁₀ linear, branched, or cyclic alkyl substituted by from 1 to 21 halogen atoms chosen from —F, —Cl, —Br, or —I;
xv) —[C(R^{69a})(R^{69b})]ᵤSO₂R⁶⁷;
   R⁶⁷ is hydrogen, hydroxyl, substituted or unsubstituted C₁-C₄ linear or branched alkyl; substituted or unsubstituted C₆, C₁₀, or C₁₄ aryl; C₇-C₁₅ alkylenearyl; C₁-C₉ substituted or unsubstituted heterocyclic; or C₁-C₁₁ substituted or unsubstituted heteroaryl;
v) two R^{d} units on the same carbon atom can be taken together to form a unit chosen from ═O, ═S, or ═NR⁶⁸;
   R⁶⁸ is hydrogen, hydroxyl, C₁-C₄ linear or branched alkyl, or C₁-C₄ linear or branched alkoxy;
      R^{69a} and R^{69b} are each independently hydrogen or C₁-C₄ alkyl; and
      the index j is an integer from 0 to 5.

The R^{d} units disclosed herein can be further substituted by one or more organic radicals independently chosen from:
i) C₁-C₁₂ linear, branched, or cyclic alkyl, alkenyl, and alkynyl;
ii) substituted or unsubstituted C₆ or C₁₀ aryl;
iii) substituted or unsubstituted C₆ or C₁₀ alkylenearyl;
iv) substituted or unsubstituted C₁-C₉ heterocyclic rings;
v) substituted or unsubstituted C₁-C₉ heteroaryl rings;
vi) —(CR^{102a}R^{102b})_{z}OR¹⁰¹;
vii) —(CR^{102a}R^{102b})_{z}C(O)R¹⁰¹;
viii) —(CR^{102a}R^{102b})_{z}C(O)OR¹⁰¹;
iv) —(CR^{102a}R^{102b})_{z}C(O)N(R¹⁰¹);
ix) —(CR^{102a}R^{102b})_{z}N(R¹⁰¹)₂;
xi) halogen;
xii) —(CR^{102a}R^{102b})_{z}CN;
xiii) —(CR^{102a}R^{102b})_{z}NO₂;
xiv) —CHⱼXₖ; wherein X is halogen, the index j is an integer from 0 to 2, j+k 3;
xv) —(CR^{102a}R^{102b})_{z}SR¹⁰¹;
xvi) —(CR^{102a}R^{102b})_{z}SO₂R¹⁰¹; and
xvii) —(CR^{102a}R^{102b})_{z}SO₃R¹⁰¹;
   wherein each R¹⁰¹ is independently hydrogen, substituted or unsubstituted C₁-C₄ linear, branched, or cyclic alkyl, phenyl, benzyl, heterocyclic, or heteroaryl; or two R¹⁰¹ units can be taken together to form a ring comprising 3-7 atoms; R^{102a} and R^{102b} are each independently hydrogen or C₁-C₄ linear or branched alkyl; the index z is from 0 to 4.

One iteration of this embodiment of R⁶⁰ relates to R⁶⁰ units that are a substituted or unsubstituted C₁, C₂, C₃, or C₄ heteroaryl or heterocyclic 5-member ring. Non-limiting examples of R⁶⁰ units are the following:
i) a pyrrolidinyl ring having the formula;

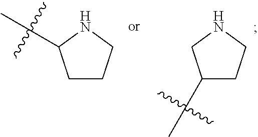

ii) a pyrrolyl ring having the formula:

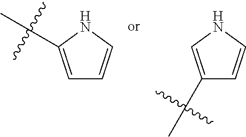

iii) a 4,5-dihydroimidazolyl ring having the formula:

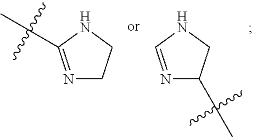

iv) a pyrazolyl ring having the formula:

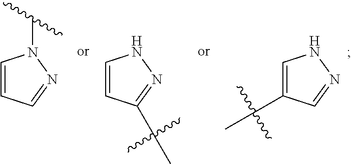

v) an imidazolyl ring having the formula:

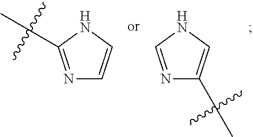

vi) a [1,2,3]triazolyl ring having the formula:

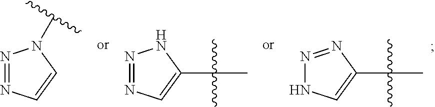

vii) a [1,2,4]triazolyl ring having the formula:

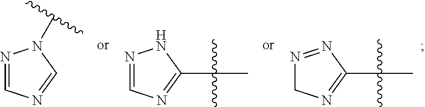

viii) tetrazolyl ring having the formula:

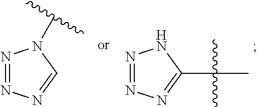

ix) a [1,3,4] or [1,2,4]oxadiazolyl ring having the formula:

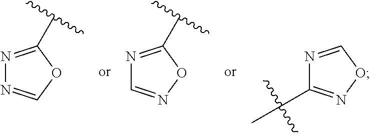

x) a pyrrolidinonyl ring having the formula:

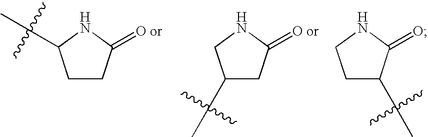

xi) an imidazolidinonyl ring having the formula:

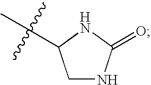

xii) an imidazol-2-only ring having the formula:

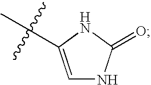

xiii) an oxazolyl ring having the formula:

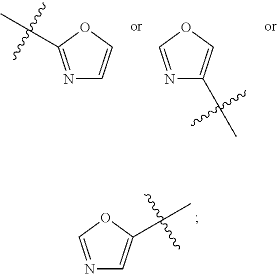

xiv) an isoxazolyl ring having the formula:

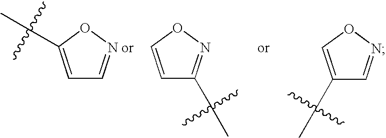

xv) a dihydrothiazolyl ring having the formula:

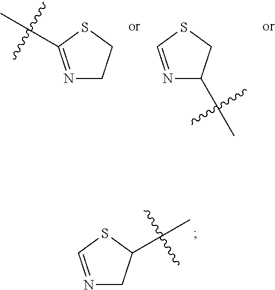

xvi) a furanly ring having the formula:

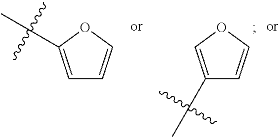

xvii) a thiophenyl having the formula:

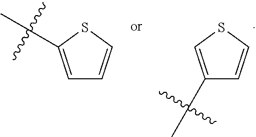

A non-limiting example of this iteration includes a compound having the formula:

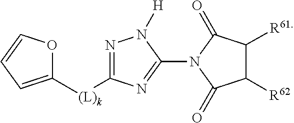

Another iteration of this embodiment of R⁶⁰ relates to R⁶⁰ units that are a substituted or unsubstituted C₃, C₄ or C₅ heterocyclic or heteroaryl 6-member ring. Non-limiting examples of R⁶⁰ units are the following:
i) a morpholinyl ring having the formula:

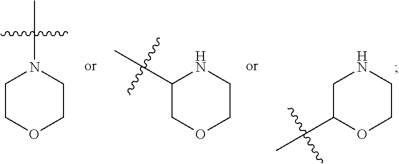

ii) a piperidinyl ring having the formula:

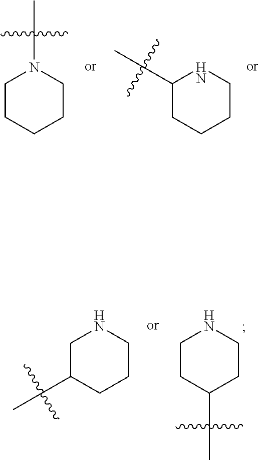

iii) a pyridinyl ring having the formula:

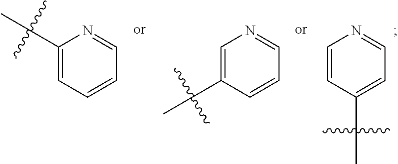

iv) a pyrimidinyl ring having the formula:

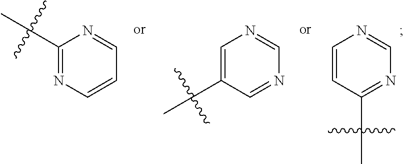

v) a piperazinyl ring having the formula:

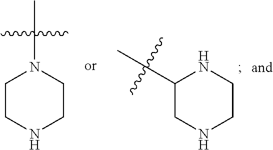

vi) a triazinyl ring having the formula:

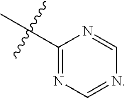

A non-limiting example of this iteration includes a compound having the formula:

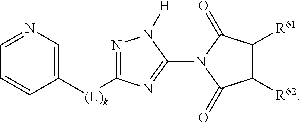

Another iteration of this embodiment of R⁶⁰ relates to R⁶⁰ units that are a substituted or unsubstituted C₇, C₈ or C₉ heterocyclic or heteroaryl fused ring. Non-limiting examples of R⁶⁰ units are the following:
i) benzoimidazolyl rings having the formula:

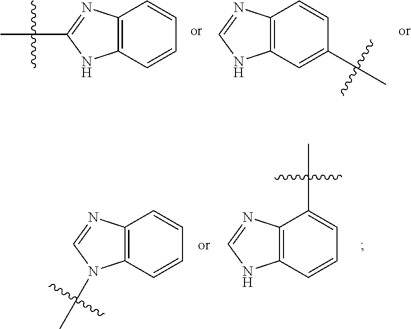

ii) benzothiazolyl rings having the formula:

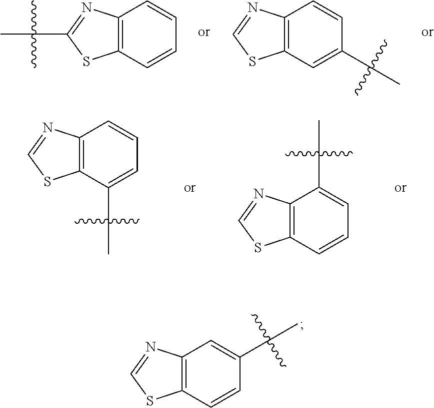

iii) benzoxazolyl rings having the formula:

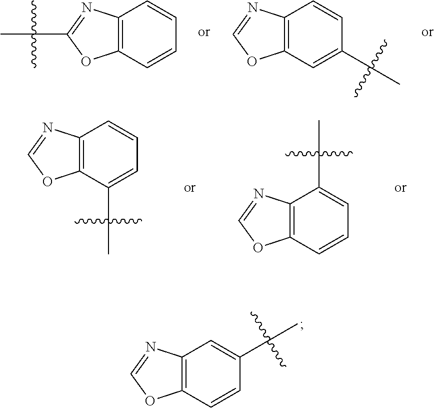

iv) quinazolinyl rings having the formula:

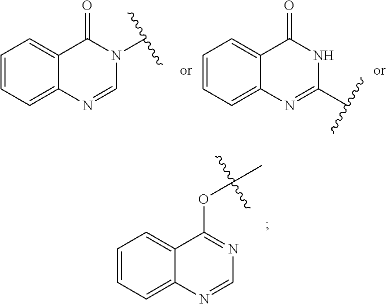

v) 2,3-dihydrobenzo[1,4]dioxinyl rings having the formula:

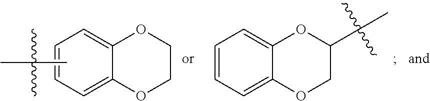

vi) tetrahydroquinolinyl rings having the formula:

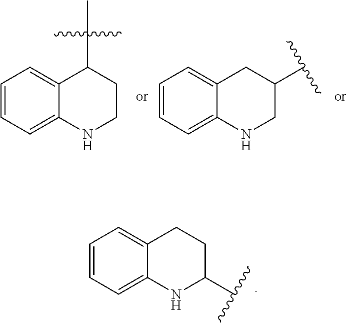

A non-limiting example of this iteration includes a compound having the formula:

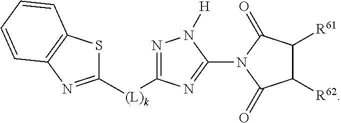

R⁶¹ and R⁶² are taken together to form a ring chosen from:
ii) saturated or unsaturated cycloalkyl having from 4-8 carbon atoms;
iii) saturated or unsaturated bicycloalkyl having from 6 to 8 carbon atoms; or
iv) C₆ or C₁₀ aryl.

In one embodiment, R⁶¹ and R⁶² are taken together to form a saturated cycloalkyl ring. The disclosed modulators according to this embodiment of R⁶¹ and R⁶² have the formula:

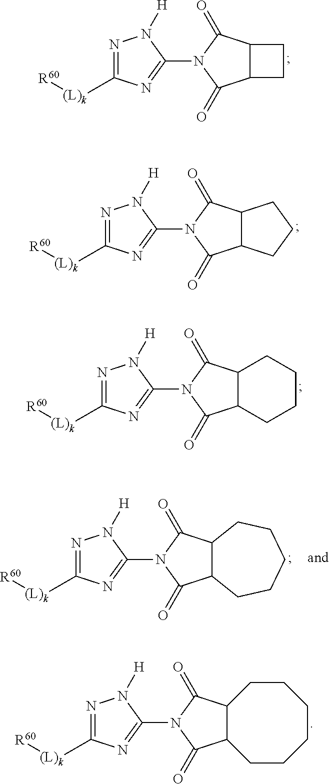

In another embodiment, R⁶¹ and R⁶² are taken together to form an unsaturated cycloalkyl ring. Non-limiting examples of the disclosed modulators according to this embodiment of R⁶¹ and R⁶² have the formula:

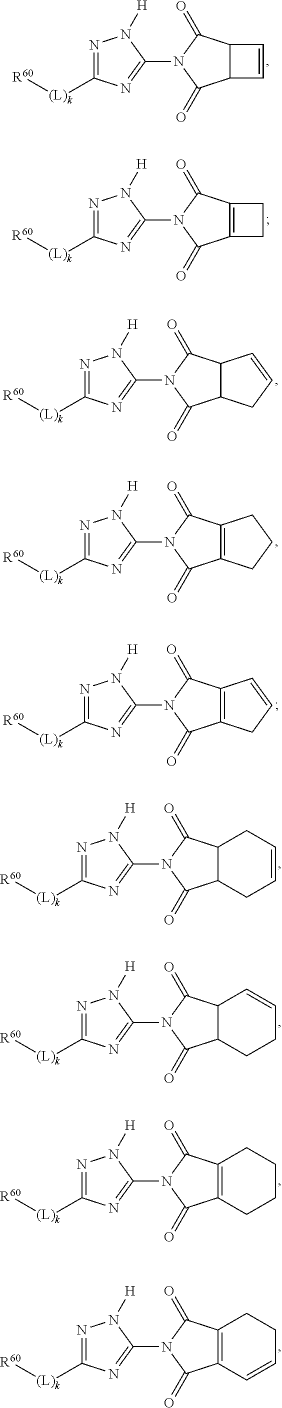

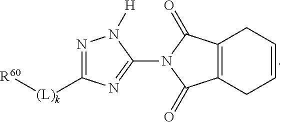

In a further embodiment, R⁶¹ and R⁶² are taken together to form a saturated cycloalkyl ring. The disclosed modulators according to this embodiment of R⁶¹ and R⁶² have the formula:

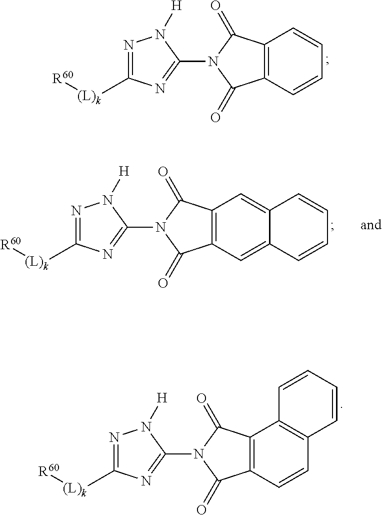

L is a linking unit having from 1 to 5 carbon atoms when L is present. The index k is equal to 1 when L is present. The index k is equal to 0 when L is absent.

One embodiment of L units relates to linear and branched alkylene units chosen from:
i) —CH₂—;
ii) —CH₂CH₂—;
iii) —CH₂CH₂CH₂—;
iv) —CH₂CH₂CH₂CH₂—;
v) —CH₂CH(CH₃)CH₂—; or
vi) —CH₂CH(CH₃)CH₂CH₂—.

One iteration of this embodiment relates to L units that are methylene (—CH₂—) units thereby providing Intestinal Alkaline Phosphatase modulators having the formula:

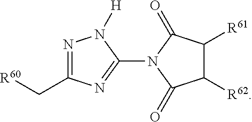

Another iteration of this embodiment relates to L units that are ethylene (—CH₂CH₂—) units thereby providing Intestinal Alkaline Phosphatase modulators having the formula:

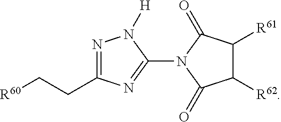

Another embodiment of L units relates to linear and branched alkenylene units chosen from:
i) —CH═CH—;
ii) —CH₂CH═CH—;
iii) —CH═CHCH₂—
iv) —CH═CHCH₂CH₂—;
v) —CH₂CH₂CH═CH—; or
vi) —CH₂CH═CHCH₂—.

One iteration of this embodiment relates to L units that are ethylene (—CH₂CH₂—) units thereby providing Intestinal Alkaline Phosphatase modulators having the formula:

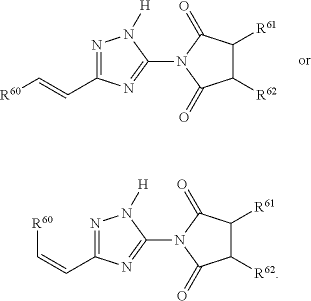

When linking unit, L, is absent the Alkaline Phosphatase modulators have the formula:

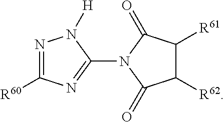

One aspect of this category of Intestinal Adrenalin Phosphatase modulators relates to compounds having a saturated ring, for example, isoindoline-1,3-dionyl compounds having the formula:

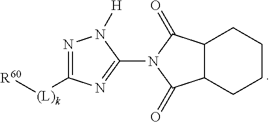

Non-limiting examples of compounds according to this aspect include:
i) 2-(1H-1,2,4-triazol-5-yl)-hexahydro-1H-isoindole-1,3(2H)-dione

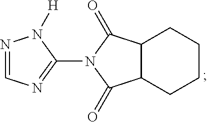

i) 2-(3-benzyl-1H-1,2,4-triazol-5-yl)-hexahydro-1H-isoindole-1,3(2H)-dione

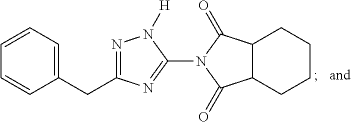

ii) 2-(3-phenethyl-1H-1,2,4-triazol-5-yl)-hexahydro-1H-isoindole-1,3(2H)-dione

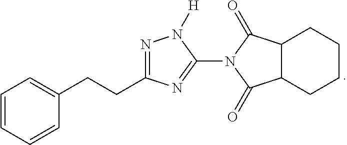

Another aspect of this category of Intestinal Adrenalin Phosphatase modulators relates to compounds having an unsaturated ring, for example, isoindole-1,3(2H)-dionyl compounds having the formula:

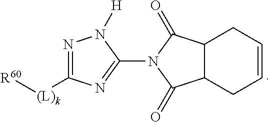

i) 2-(1H-1,2,4-triazol-5-yl)-3a,4,7,7a-tetrahydro-1H-isoindole-1 ,3(2H)-dione

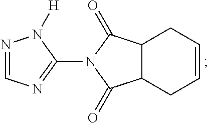

ii) 2-(3-phenyl-1H-1,2,4-triazol-5-yl)-3a,4,7,7a-tetrahydro-1H-isoindole-1,3(2H)-dione

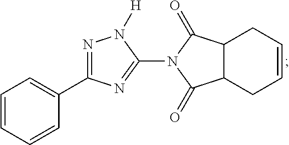

iii) 2-[3-(furan-2-yl)-1H-1,2,4-triazol-5-yl]-3a,4,7,7a-tetrahydro-1H-isoindole-1,3(2H)-dione

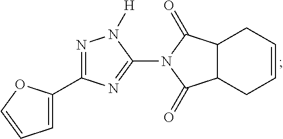

iv) 2-[3-(pyridin-3-yl)-1H-1,2,4-triazol-5-yl)-3a,4,7,7a-tetrahydro-1H-isoindole-1,3(2H)-dione

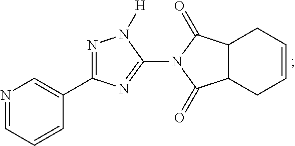

v) 2-(3-benzyl-1H-1,2,4-triazol-5-yl)-3a,4,7,7a-tetrahydro-1H-isoindole-1,3(2H)-dione

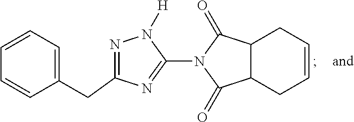

vi) 2-(3-phenethyl-1H-1,2,4-triazol-5-yl)-3a,4,7,7a-tetrahydro-1H-isoindole-1,3(2H)-dione

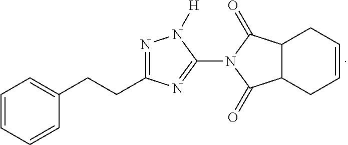

In addition, the compounds of this category can comprise bicyclic rings, for example, the compound having the formula:

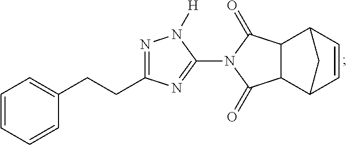

A further aspect of this category of Intestinal Adrenalin Phosphatase modulators relates to compounds having an unsaturated ring, for example, isoindoline-1,3-dionyl compounds having the formula:

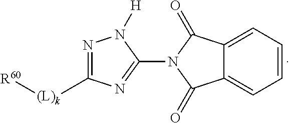

A non-limiting example of this aspect includes 2-(3-benzyl-1H-1,2,4-triazol-5-yl)isoindoline-1,3-dione having the formula:

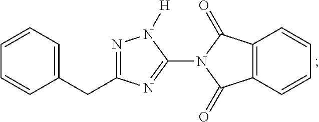

A further example of compounds according to this category include relates to N-aryl substituted 1H-1,2,4-triazoles, for example, 2-(5-amino-1-phenyl-1H-1,2,4-triazol-3-yl)-3a,4,7,7a-tetrahydro-1H-isoindole-1,3(2H)-dione having the formula:

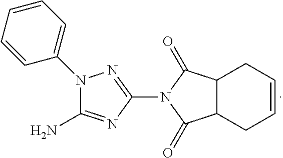

Table F provides non-limiting examples of Intestinal Alkaline Phosphatase activators and inhibitors according to this category.

TABLE F
IAP modulators (F)

| | | IC₅₀ | |
|---|---|---|---|
| No. | Compound | (μM) | n* |
| F1 | 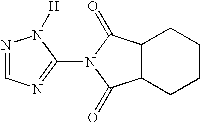 | 13.8 | −2.03 |
| | 2-(1H-1,2,4-triazol-5-yl)-hexahydro-1H- | | |
| | isoindole-1,3(2H)-dione | | |
| F2 | 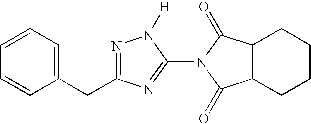 | 0.0613 | 1.01 |
| | 2-(3-benzyl-1H-1,2,4-triazol-5-yl)-hexahydro- | | |
| | 1H-isoindole-1,3(2H)-dione | | |
| F3 | 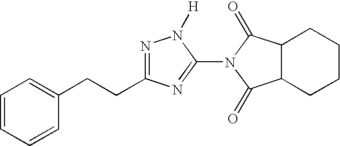 | 1.75 | 0.694 |
| | 2-(3-phenethyl-1H-1,2,4-triazol-5-yl)-hexahydro- | | |
| | 1H-isoindole-1,3(2H)-dione | | |
| F4 | 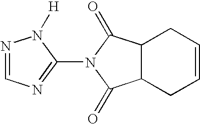 | 34.5 | −1.61 |
| | 2-(1H-1,2,4-triazol-5-yl)-3a,4,7,7a-tetrahydro-1H- | | |
| | isoindole-1,3(2H)-dione | | |
| F5 | 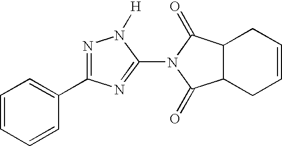 | 0.0625 | 0.8895 |
| | 2-(3-phenyl-1H-1,2,4-triazol-5-yl)-3a,4,7,7a- | | |
| | tetrahydro-1H-isoindole-1,3(2H)-dione | | |
| F6 | 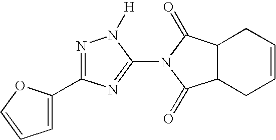 | 0.411 | 0.894 |
| | 2-[3-(furan-2-yl)-1H-1,2,4-triazol-5-yl]- | | |
| | 3a,4,7,7a-tetrahydro-1H-isoindole-1,3(2H)- | | |
| | dione | | |
| F7 | 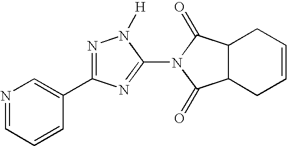 | 1.77 | 0.927 |
| | 2-[3-(pyridin-3-yl)-1H-1,2,4-triazol-5-yl)- | | |
| | 3a,4,7,7a-tetrahydro-1H-isoindole-1,3(2H)- | | |
| | dione | | |
| F8 | 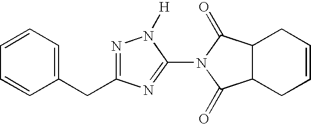 | 0.5035 | 1.28 |
| | 2-(3-benzyl-1H-1,2,4-triazol-5-yl)-3a,4,7,7a- | | |
| | tetrahydro-1H-isoindole-1,3(2H)-dione | | |
| F9 | 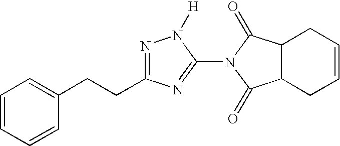 | 0.724 | 0.616 |
| | 2-(3-phenethyl-1H-1,2,4-triazol-5-yl)-3a,4,7,7a- | | |
| | tetrahydro-1H-isoindole-1,3(2H)-dione | | |
| F10 | 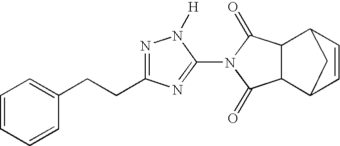 | 4.75 | 0.871 |
| F11 | 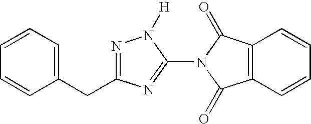 | 8.865 | 0.5445 |
| | 2-(3-benzyl-1H-1,2,4-triazol-5-yl)isoindoline- | | |
| | 1,3-dione | | |
| F12 | 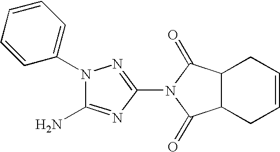 | >100 | — |
| | 2-(5-amino-1-phenyl-1H-1,2,4-triazol-3-yl)- | | |
| | 3a,4,7,7a-tetrahydro-1H-isoindole-1,3(2H)- | | |
| | dione | | |

A further category of Intestinal Alkaline Phosphatase modulators relates to modulators having the formula:

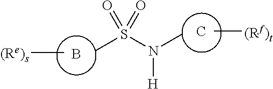

wherein B and C are a ring independently chosen from:
i) C₆ or C₁₀ aryl; or
ii) C₁-C₉ heteroaryl;
   R^{e} and R^{f} represent from 1 to 9 substitutions for hydrogen on the B and C rings respectively and each R^{e} and R^{f} is independently chosen from:
i) substituted or unsubstituted C₁-C₁₀ linear, branched or cyclic alkyl;
ii) substituted or unsubstituted C₂-C₁₀ linear, branched or cyclic alkenyl;
iii) substituted or unsubstituted C₂-C₁₀ linear or branched or alkynyl;
iv) substituted or unsubstituted C₁-C₁₀ linear, branched or cyclic alkoxy;
v) substituted or unsubstituted C₂-C₁₀ linear, branched or cyclic alkenoxy;
vi) substituted or unsubstituted C₂-C₁₀ linear or branched alkynoxy;
vii) halogen; or
viii) hydroxy;
   the index s is an integer from 0 to 9; and the index t is an integer from 0 to 9. The indices or t are equal to 0, there are no substitutions for hydrogen on the corresponding ring.

One aspect of B and C rings relates to C₁-C₉ heteroaryl rings. A first embodiment of this aspect relates to substituted or unsubstituted C₁, C₂, C₃, or C₄ heteroaryl 5-member ring having a formula chosen from:

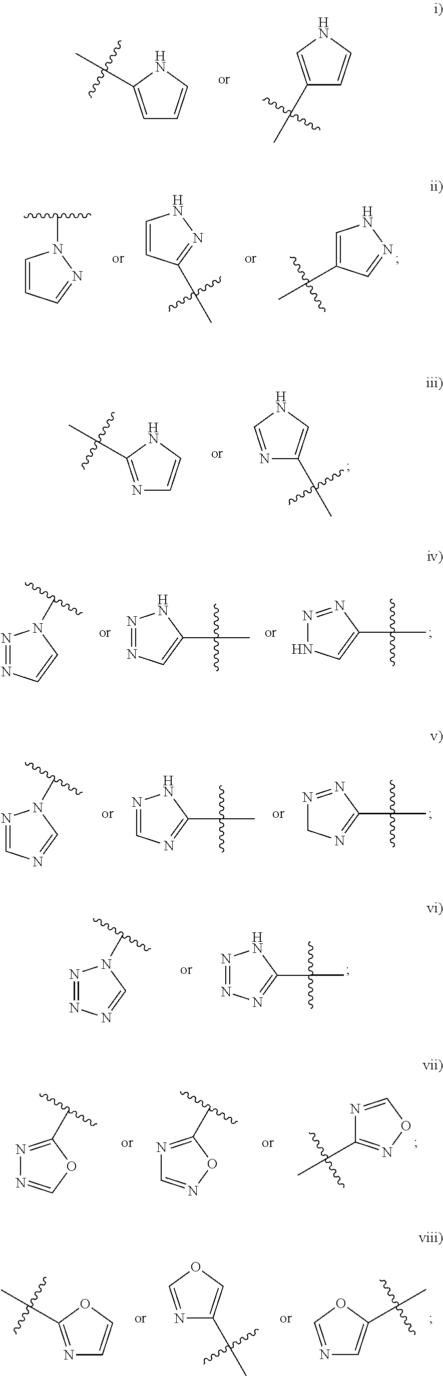

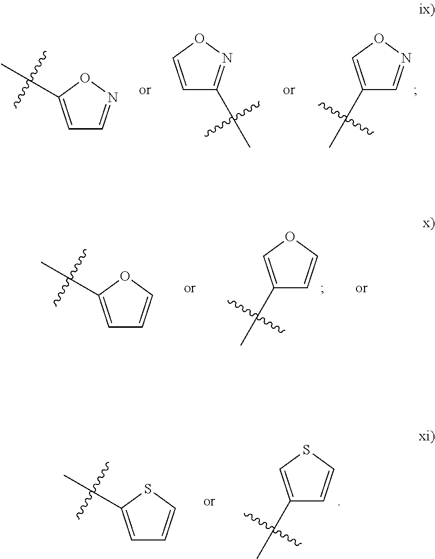

A further embodiment relates to C₃, C₄, or C₅ heteroaryl 6-member rings having a formula chosen from:

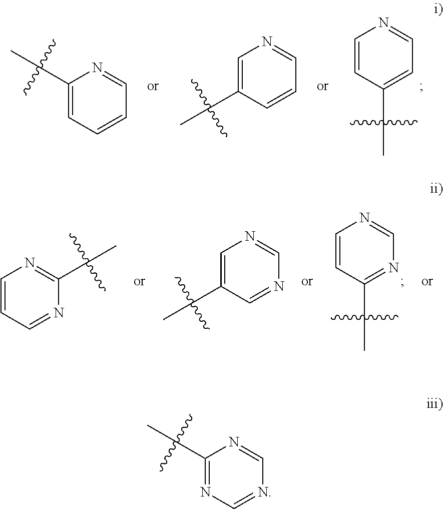

The first aspect of B rings relates to compounds wherein B is substituted or unsubstituted C₆ aryl (phenyl) or C₁₀ aryl (naphthalen-1-yl or naphthalen-2-yl). One embodiment of this aspect relates to B rings that are unsubstituted C₆ (phenyl) thereby providing compounds having the formula:

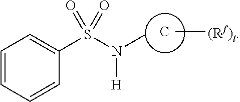

The following are non-limiting iterations of compounds according to this embodiment:
i) substituted or unsubstituted N-(phenyl)benzenesulfonamides:

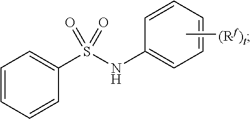

ii) substituted or unsubstituted N-(pyridin-3-yl)benzenesulfonamides:

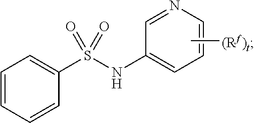

iii) substituted or unsubstituted N-(pyrazin-2-yl)benzenesulfonamides:

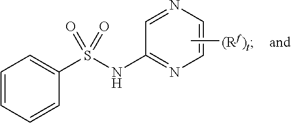

iv) substituted or unsubstituted N-(quinolin-3-yl)benzenesulfonamides:

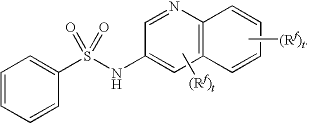

The following are non-limiting examples of compounds according to this aspect:

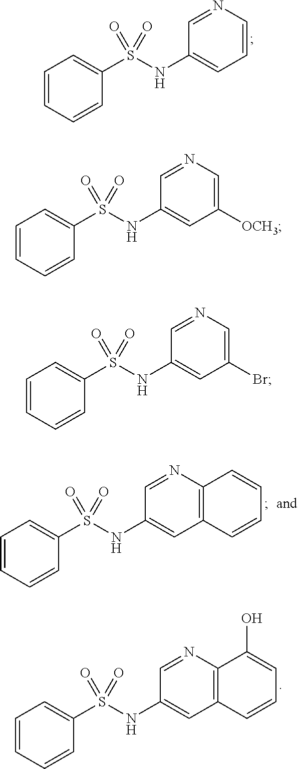

Another embodiment of this aspect relates to B rings that are substituted or unsubstituted phenyl. Non-limiting examples of substitutions on the B phenyl ring include:
i) C₁-C₆ linear, branched, or cyclic alkyl, alkenyl, and alkynyl; for example, methyl (C₁), ethyl (C₂), ethenyl (C₂), ethynyl (C₂), n-propyl (C₃), iso-propyl (C₃), cyclopropyl (C₃), 3-propenyl (C₃), 1-propenyl (also 2-methylethenyl) (C₃), isopropenyl (also 2-methylethen-2-yl) (C₃), prop-2-ynyl (also propargyl) (C₃), propyn-1-yl (C₃), n-butyl (C₄), sec-butyl (C₄), iso-butyl (C₄), tert-butyl (C₄), cyclobutyl (C₄), buten-4-yl (C₄), cyclopentyl (C₅), and cyclohexyl (C₆);
ii) —(CR^{102a}R^{102b})_{z}OR¹⁰¹; for example, —OH, —CH₂OH, —OCH₃, —CH₂OCH₃, —OCH₂CH₃, —CH₂OCH₂CH₃, —OCH₂CH₂CH₃, and —CH₂OCH₂CH₂CH₃; and
iii) halogen; —F, —Cl, —Br, and —I;
   wherein each R¹⁰¹ is independently hydrogen, substituted or unsubstituted C₁-C₄ linear, branched, or cyclic alkyl, phenyl, benzyl, heterocyclic, or heteroaryl; or two R¹⁰¹ units can be taken together to form a ring comprising 3-7 atoms; R^{102a} and R^{102b} are each independently hydrogen or C₁-C₄ linear or branched alkyl; the index z is from 0 to 4.

The following are non-limiting iterations of compounds according to this embodiment:
i) substituted or unsubstituted N-(phenyl)(substituted)benzenesulfonamides:

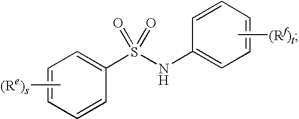

ii) substituted or unsubstituted N-(pyridin-3-yl)(substituted)benzenesulfonamides:

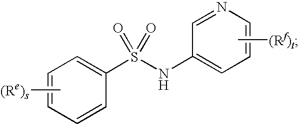

iii) substituted or unsubstituted N-(pyrazin-2-yl)(substituted)benzenesulfonamides:

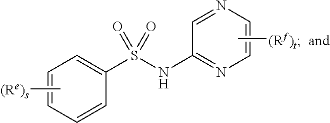

iv) substituted or unsubstituted N-(quinolin-3-yl)(substituted)benzenesulfonamides:

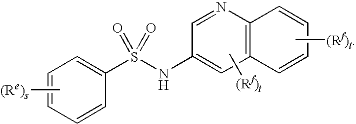

Non-limiting examples of compounds according to this embodiment include:
i) 5-bromo-2-methoxy-N-(pyridin-3-yl)benzenesulfonamide:

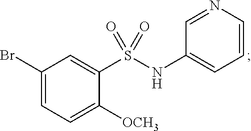

ii) 5-bromo-2-methoxy-N-(quinolin-3-yl)benzenesulfonamide:

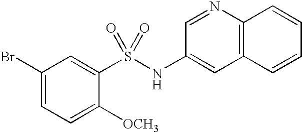

iii) 5-bromo-2-methoxy-N-(quinoxalin-2-yl)benzenesulfonamide:

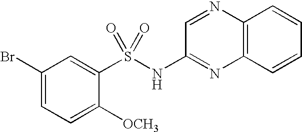

iv) 2,5-dimethoxy-N-(pyrazin-2-yl)benzenesulfonamide:

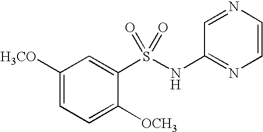

v) 2,5-dimethoxy-N-(quinolin-3-yl)benzenesulfonamide:

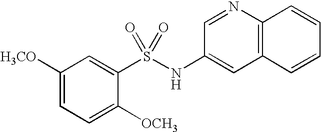

vi) 2,5-dimethoxy-N-(quinoxalin-2-yl)benzenesulfonamide:

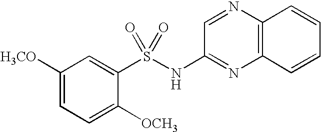

vii) 5-chloro-2-ethoxy-N-(quinoxalin-2-yl)benzenesulfonamide:

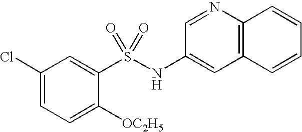

viii) 5-chloro-2-ethoxy-N-(pyridin-3-yl)benzenesulfonamide:

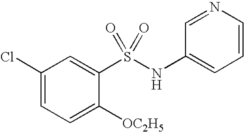

ix) 5-chloro-2-ethoxy-N-(quinoxalin-2-yl)benzenesulfonamide:

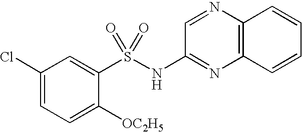

x) 2-methyl-N-(pyridin-3-yl)benzenesulfonamide:

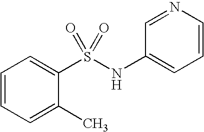

xi) 2-methyl-N-(quinolin-3-yl)benzenesulfonamide:

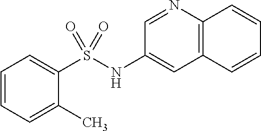

xii) 2-methyl-N-(quinoxalin-3-yl)benzenesulfonamide:

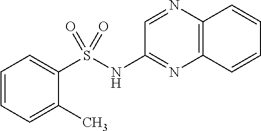

xiii) 2-methoxy-4-methyl-5-chloro-N-(pyridin-3-yl)benzenesulfonamide:

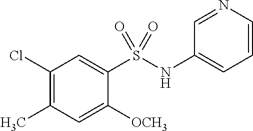

xiv) 2-methoxy-4-methyl-5-chloro-N-(quinolin-3-yl)benzenesulfonamide:

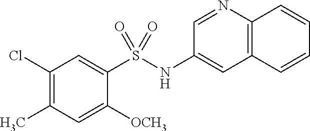

xv) 2-methoxy-4-methyl-5-chloro-N-(quinoxalin-2-yl)benzenesulfonamide:

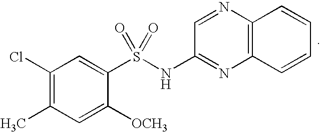

Table G provides non-limiting examples of Intestinal Alkaline Phosphatase activators and inhibitors according to this category.

TABLE G
IAP modulators (G)

| | | IC₅₀ | |
|---|---|---|---|
| No. | Compound | (μM) | n* |
| G1 | 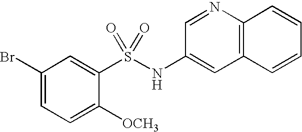 | 51.5 | 0.716 |
| | 5-bromo-2-methoxy-N-(quinolin-3- | | |
| | yl)benzenesulfonamide | | |
| G2 | 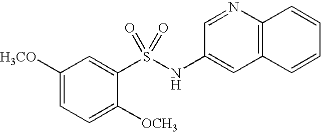 | >100 | — |
| | 2,5-dimethoxy-N-(quinolin-3- | | |
| | yl)benzenesulfonamide | | |
| G3 | 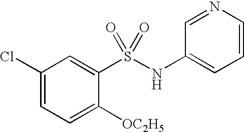 | >100 | — |
| | 5-chloro-2-ethoxy-N-(pyridin-3- | | |
| | yl)benzenesulfonamide | | |
| G4 | 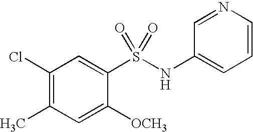 | >100 | — |
| | 2-methoxy-4-methyl-5-chloro-N-(pyridin-3- | | |
| | yl)benzenesulfonamide | | |
| G5 | 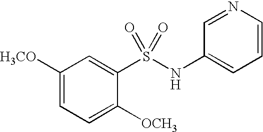 | >100 | — |
| | 2,5-dimethoxy-N-(pyridin-2- | | |
| | yl)benzenesulfonamide | | |
| G6 | 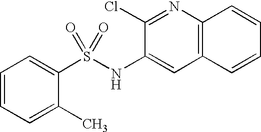 | >100 | — |
| | N-(2-chloroquinolin-3-yl)-2- | | |
| | methylbenzenesulfonamide | | |

Table H provides further non-limiting examples of Intestinal Alkaline Phosphatase activators and inhibitors.

TABLE H
IAP modulators (H)

| | | IC₅₀ | |
|---|---|---|---|
| No. | Compound | (μM) | n* |
| H1 | 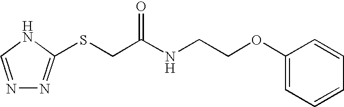 | >100 | — |
| | 2-(4H-1,2,4-triazol-3-ylthio)-N-(2- | | |
| | phenoxyethyl)acetamide | | |
| H2 | 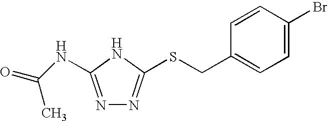 | 86.4 | −0.563 |
| | N-[5-(4-bromobenzylthio)-4H-1,2,4-triazol-3- | | |
| | yl)acetamide | | |
| H3 | 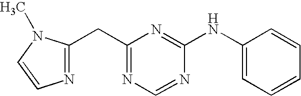 | 30.5 | −1.32 |
| | 4-[(1-methyl-4H-imidazol-2-yl)methyl]-N-phenyl- | | |
| | 1,3,5-triazin-2-amine | | |
| H4 | 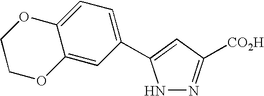 | >100 | — |
| | 5-(2,3-dihydrobenzo[b][1,4]dioxin-6-yl)-1H- | | |
| | pyrazole-3-carboxylic acid | | |
| H5 | 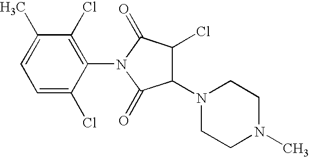 | >100 | — |
| | 3-chloro-1-(2,6-dichloro-3-methylphenyl)-4-(4- | | |
| | methylpiperazin-1-yl)pyrrolidine-2,5-dione | | |

2. Formulations

Disclosed herein are compositions that comprise one or more of the disclosed compounds, for example, a composition comprising: an effective amount of one or more intestinal alkaline phosphatase modulators as disclosed herein; and a pharmaceutically acceptable carrier.

Further disclosed are compositions comprising: an effective amount of one or more intestinal alkaline phosphatase activators as disclosed herein; and a pharmaceutically acceptable carrier.

Also disclosed are compositions comprising: an effective amount of one or more intestinal alkaline phosphatase inhibitors as disclosed herein; and a pharmaceutically acceptable carrier.

Those skilled in the art based upon the present description and the nature of any given inhibitor identified by the assays disclosed herein will understand how to determine a therapeutically effective dose thereof.

The pharmaceutical compositions can be manufactured using any suitable means, e.g., by means of conventional mixing, dissolving, granulating, dragee-making, levigating, emulsifying, encapsulating, entrapping or lyophilizing processes.

Pharmaceutical compositions for use in accordance with the present disclosure thus can be formulated in a conventional manner using one or more physiologically or pharmaceutically acceptable carriers (vehicles, or diluents) comprising excipients and auxiliaries which facilitate processing of the active compounds into preparations which can be used pharmaceutically. Proper formulation is dependent upon the route of administration chosen.

Any suitable method of administering a pharmaceutical composition to a subject can be used in the disclosed treatment method, including injection, transmucosal, oral, inhalation, ocular, rectal, long acting implantation, liposomes, emulsion, or sustained release means.

For injection, the disclosed agents can be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the disclosed compounds to be formulated as tablets, pills, dragees, capsules, liquids, gels, syrups, slurries, suspensions and the like, for oral ingestion by a patient to be treated. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl-pyrrolidone (PVP). If desired, disintegrating agents can be added, such as cross-linked polyvinylpyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of tablets or lozenges formulated in conventional manner.

For administration by inhalation, the disclosed compounds can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin, for use in an inhaler or insufflator, can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions.

Alternatively, the active ingredient can be in powder form for constitution with a suitable vehicle, such as sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

One type of pharmaceutical carrier for hydrophobic compounds is a cosolvent system comprising benzyl alcohol, a nonpolar surfactant, a water-miscible organic polymer, and an aqueous phase.

The cosolvent system can be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system can be varied considerably without destroying its solubility and toxicity characteristics. Furthermore, the identity of the co-solvent components can be varied: for example, other low-toxicity nonpolar surfactants can be used instead of polysorbate 80; the fraction size of polyethylene glycol can be varied; other biocompatible polymers can replace polyethylene glycol, e.g., polyvinyl pyrrolidone; and other sugars or polysaccharides can be substituted for dextrose.

Alternatively, other delivery systems for hydrophobic pharmaceutical compounds can be employed. Liposomes and emulsions are well known examples of delivery vehicles or carriers for hydrophobic drugs. Certain organic solvents such as dimethylsulfoxide also can be employed.

Additionally, the compounds can be delivered using any suitable sustained-release system, such as semipermeable matrices of solid hydrophobic polymers containing the therapeutic agent. Various sustained-release materials have been established and are well known by those skilled in the art. Sustained-release capsules can, depending on their chemical nature, release the compounds for a prolonged period of time. Depending on the chemical nature and the biological stability of the therapeutic reagent, additional strategies for protein stabilization can be employed.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the disclosed agents can be provided as salts with pharmaceutically acceptable counterions. Salts tend to be more soluble in aqueous or other protonic solvents than are the corresponding free base forms.

Also disclosed are methods of treating a condition or a disease in a mammal comprising administering to said mammal a pharmaceutical composition disclosed herein.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

The disclosed IAP modulator can be combined, conjugated or coupled with or to carriers and other compositions to aid administration, delivery or other aspects of the inhibitors and their use. For convenience, such composition are referred to herein as carriers. Carriers can, for example, be a small molecule, pharmaceutical drug, fatty acid, detectable marker, conjugating tag, nanoparticle, or enzyme.

The disclosed compositions can be used therapeutically in combination with a pharmaceutically acceptable carrier. By “pharmaceutically acceptable” is meant a material that is not biologically or otherwise undesirable, i.e., the material can be administered to a subject, along with the composition, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A.R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers can be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds can be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration include powders or granules, suspensions or solutions in water or non-aqueous media, capsules, sachets, or tablets. Thickeners, flavorings, diluents, emulsifiers, dispersing aids or binders can be desirable.

Some of the compositions can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

The term “nanoparticle” refers to a nanoscale particle with a size that is measured in nanometers, for example, a nanoscopic particle that has at least one dimension of less than about 100 nm. Examples of nanoparticles include paramagnetic nanoparticles, superparamagnetic nanoparticles, metal nanoparticles, fullerene-like materials, inorganic nanotubes, dendrimers (such as with covalently attached metal chelates), nanofibers, nanohoms, nano-onions, nanorods, nanoropes and quantum dots. A nanoparticle can produce a detectable signal, for example, through absorption and/or emission of photons (including radio frequency and visible photons) and plasmon resonance.

Microspheres (or microbubbles) can also be used with the methods disclosed herein. Microspheres containing chromophores have been utilized in an extensive variety of applications, including photonic crystals, biological labeling, and flow visualization in microfluidic channels. See, for example, Y. Lin, et al., Appl. Phys Lett. 2002, 81, 3134; D. Wang, et al., Chem. Mater. 2003, 15, 2724; X. Gao, et al., J. Biomed. Opt. 2002, 7, 532; M. Han, et al., Nature Biotechnology. 2001, 19, 631; V. M. Pai, et al., Mag. & Magnetic Mater. 1999, 194, 262, each of which is incorporated by reference in its entirety. Both the photostability of the chromophores and the monodispersity of the microspheres can be important.

Nanoparticles, such as, for example, silica nanoparticles, metal nanoparticles, metal oxide nanoparticles, or semiconductor nanocrystals can be incorporated into microspheres. The optical, magnetic, and electronic properties of the nanoparticles can allow them to be observed while associated with the microspheres and can allow the microspheres to be identified and spatially monitored. For example, the high photostability, good fluorescence efficiency and wide emission tunability of colloidally synthesized semiconductor nanocrystals can make them an excellent choice of chromophore. Unlike organic dyes, nanocrystals that emit different colors (i.e. different wavelengths) can be excited simultaneously with a single light source. Colloidally synthesized semiconductor nanocrystals (such as, for example, core-shell CdSe/ZnS and CdS/ZnS nanocrystals) can be incorporated into microspheres. The microspheres can be monodisperse silica microspheres.

The nanoparticle can be a metal nanoparticle, a metal oxide nanoparticle, or a semiconductor nanocrystal. The metal of the metal nanoparticle or the metal oxide nanoparticle can include titanium, zirconium, hafnium, vanadium, niobium, tantalum, chromium, molybdenum, tungsten, manganese, technetium, rhenium, iron, ruthenium, osmium, cobalt, rhodium, iridium, nickel, palladium, platinum, copper, silver, gold, zinc, cadmium, scandium, yttrium, lanthanum, a lanthanide series or actinide series element (e.g., cerium, praseodymium, neodymium, promethium, samarium, europium, gadolinium, terbium, dysprosium, holmium, erbium, thulium, ytterbium, lutetium, thorium, protactinium, and uranium), boron, aluminum, gallium, indium, thallium, silicon, germanium, tin, lead, antimony, bismuth, polonium, magnesium, calcium, strontium, and barium. In certain embodiments, the metal can be iron, ruthenium, cobalt, rhodium, nickel, palladium, platinum, silver, gold, cerium or samarium. The metal oxide can be an oxide of any of these materials or combination of materials. For example, the metal can be gold, or the metal oxide can be an iron oxide, a cobalt oxide, a zinc oxide, a cerium oxide, or a titanium oxide. Preparation of metal and metal oxide nanoparticles is described, for example, in U.S. Pat. Nos. 5,897,945 and 6,759,199, each of which is incorporated by reference in its entirety.

For example, the disclosed compounds can be immobilized on silica nanoparticles (SNPs). SNPs have been widely used for biosensing and catalytic applications owing to their favorable surface area-to-volume ratio, straightforward manufacture and the possibility of attaching fluorescent labels, magnetic nanoparticles (Yang, H. H. et al. 2005) and semiconducting nanocrystals (Lin, Y. W., et al. 2006).

The nanoparticle can also be, for example, a heat generating nanoshell. As used herein, “nanoshell” is a nanoparticle having a discrete dielectric or semi-conducting core section surrounded by one or more conducting shell layers. U.S. Pat. No. 6,530,944 is hereby incorporated by reference herein in its entirety for its teaching of the methods of making and using metal nanoshells.

Targeting molecules can be attached to the disclosed compositions and/or carriers. For example, the targeting molecules can be antibodies or fragments thereof, ligands for specific receptors, or other proteins specifically binding to the surface of the cells to be targeted.

“Liposome” as the term is used herein refers to a structure comprising an outer lipid bi- or multi-layer membrane surrounding an internal aqueous space. Liposomes can be used to package any biologically active agent for delivery to cells.

Materials and procedures for forming liposomes are well-known to those skilled in the art. Upon dispersion in an appropriate medium, a wide variety of phospholipids swell, hydrate and form multilamellar concentric bilayer vesicles with layers of aqueous media separating the lipid bilayers. These systems are referred to as multilamellar liposomes or multilamellar lipid vesicles (“MLVs”) and have diameters within the range of 10 nm to 100 μm. These MLVs were first described by Bangham, et al., J. Mol. Biol. 13:238-252 (1965). In general, lipids or lipophilic substances are dissolved in an organic solvent. When the solvent is removed, such as under vacuum by rotary evaporation, the lipid residue forms a film on the wall of the container. An aqueous solution that typically contains electrolytes or hydrophilic biologically active materials is then added to the film. Large MLVs are produced upon agitation. When smaller MLVs are desired, the larger vesicles are subjected to sonication, sequential filtration through filters with decreasing pore size or reduced by other forms of mechanical shearing. There are also techniques by which MLVs can be reduced both in size and in number of lamellae, for example, by pressurized extrusion (Barenholz, et al., FEBS Lett. 99:210-214 (1979)).

Liposomes can also take the form of unilamnellar vesicles, which are prepared by more extensive sonication of MLVs, and consist of a single spherical lipid bilayer surrounding an aqueous solution. Unilamellar vesicles (“ULVs”) can be small, having diameters within the range of 20 to 200 nm, while larger ULVs can have diameters within the range of 200 nm to 2 μm. There are several well-known techniques for making unilamellar vesicles. In Papahadjopoulos, et al., Biochim et Biophys Acta 135:624-238 (1968), sonication of an aqueous dispersion of phospholipids produces small ULVs having a lipid bilayer surrounding an aqueous solution. Schneider, U.S. Pat. No. 4,089,801 describes the formation of liposome precursors by ultrasonication, followed by the addition of an aqueous medium containing amphiphilic compounds and centrifugation to form a biomolecular lipid layer system.

Small ULVs can also be prepared by the ethanol injection technique described by Batzri, et al., Biochim et Biophys Acta 298:1015-1019 (1973) and the ether injection technique of Deamer, et al., Biochim et Biophys Acta 443:629-634 (1976). These methods involve the rapid injection of an organic solution of lipids into a buffer solution, which results in the rapid formation of unilamellar liposomes. Another technique for making ULVs is taught by Weder, et al. in “Liposome Technology”, ed. G. Gregoriadis, CRC Press Inc., Boca Raton, Fla., Vol. I, Chapter 7, pg. 79-107 (1984). This detergent removal method involves solubilizing the lipids and additives with detergents by agitation or sonication to produce the desired vesicles.

Papahadjopoulos, et al., U.S. Pat. No. 4,235,871, describes the preparation of large ULVs by a reverse phase evaporation technique that involves the formation of a water-in-oil emulsion of lipids in an organic solvent and the drug to be encapsulated in an aqueous buffer solution. The organic solvent is removed under pressure to yield a mixture which, upon agitation or dispersion in an aqueous media, is converted to large ULVs. Suzuki et al., U.S. Pat. No. 4,016,100, describes another method of encapsulating agents in unilamellar vesicles by freezing/thawing an aqueous phospholipid dispersion of the agent and lipids.

In addition to the MLVs and ULVs, liposomes can also be multivesicular. Described in Kim, et al., Biochim et Biophys Acta 728:339-348 (1983), these multivesicular liposomes are spherical and contain internal granular structures. The outer membrane is a lipid bilayer and the internal region contains small compartments separated by bilayer septum. Still yet another type of liposomes are oligolamellar vesicles (“OLVs”), which have a large center compartment surrounded by several peripheral lipid layers. These vesicles, having a diameter of 2-15 μm, are described in Callo, et al., Cryobiology 22(3):251-267 (1985).

Mezei, et al., U.S. Pat. Nos. 4,485,054 and 4,761,288 also describe methods of preparing lipid vesicles. More recently, Hsu, U.S. Pat. No. 5,653,996 describes a method of preparing liposomes utilizing aerosolization and Yiournas, et al., U.S. Pat. No. 5,013,497 describes a method for preparing liposomes utilizing a high velocity-shear mixing chamber. Methods are also described that use specific starting materials to produce ULVs (Wallach, et al., U.S. Pat. No. 4,853,228) or OLVs (Wallach, U.S. Pat. Nos. 5,474,848 and 5,628,936).

A comprehensive review of all the aforementioned lipid vesicles and methods for their preparation are described in “Liposome Technology”, ed. G. Gregoriadis, CRC Press Inc., Boca Raton, Fla., Vol. I, II & III (1984). This and the aforementioned references describing various lipid vesicles suitable for use herein are incorporated herein by reference.

Fatty acids (i.e., lipids) that can be conjugated to the provided compositions include those that allow the efficient incorporation of the proprotein convertase inhibitors into liposomes. Generally, the fatty acid is a polar lipid. Thus, the fatty acid can be a phospholipid The provided compositions can comprise either natural or synthetic phospholipid. The phospholipids can be selected from phospholipids containing saturated or unsaturated mono or disubstituted fatty acids and combinations thereof. These phospholipids can be dioleoylphosphatidylcholine, dioleoylphosphatidylserine, dioleoylphosphatidylethanolamine, dioleoylphosphatidylglycerol, dioleoylphosphatidic acid, palmitoyloleoylphosphatidylcholine, palmitoyloleoylphosphatidylserine, palmitoyloleoylphosphatidylethanolamine, palmitoyloleoylphophatidylglycerol, palmitoyloleoylphosphatidic acid, palmitelaidoyloleoylphosphatidylcholine, palmitelaidoyloleoylphosphatidylserine, palmitelaidoyloleoylphosphatidylethanolamine, palmitelaidoyloleoylphosphatidylglycerol, palmitelaidoyloleoylphosphatidic acid, myristoleoyloleoylphosphatidylcholine, myristoleoyloleoylphosphatidylserine, myristoleoyloleoylphosphatidylethanoamine, myristoleoyloleoylphosphatidylglycerol, myristoleoyloleoylphosphatidic acid, dilinoleoylphosphatidylcholine, dilinoleoylphosphatidylserine, dilinoleoylphosphatidylethanolamine, dilinoleoylphosphatidylglycerol, dilinoleoylphosphatidic acid, palmiticlinoleoylphosphatidylcholine, palmiticlinoleoylphosphatidylserine, palmiticlinoleoylphosphatidylethanolamine, palmiticlinoleoylphosphatidylglycerol, palmiticlinoleoylphosphatidic acid. These phospholipids can also be the monoacylated derivatives of phosphatidylcholine (lysophophatidylidylcholine), phosphatidylserine (lysophosphatidylserine), phosphatidylethanolamine (lysophosphatidylethanolamine), phophatidylglycerol (lysophosphatidylglycerol) and phosphatidic acid (lysophosphatidic acid). The monoacyl chain in these lysophosphatidyl derivatives can be palimtoyl, oleoyl, palmitoleoyl, linoleoyl myristoyl or myristoleoyl. The phospholipids can also be synthetic. Synthetic phospholipids are readily available commercially from various sources, such as AVANTI Polar Lipids (Albaster, Ala.); Sigma Chemical Company (St. Louis, Mo.). These synthetic compounds can be varied and can have variations in their fatty acid side chains not found in naturally occurring phospholipids. The fatty acid can have unsaturated fatty acid side chains with C14, C16, C18 or C20 chains length in either or both the PS or PC. Synthetic phospholipids can have dioleoyl (18:1)-PS; palmitoyl (16:0)-oleoyl (18:1)-PS, dimyristoyl (14:0)-PS; dipalmitoleoyl (16:1)-PC, dipalmitoyl (16:0)-PC, dioleoyl (18:1)-PC, palmitoyl (16:0)-oleoyl (18:1)-PC, and myristoyl (14:0)-oleoyl (18:1)-PC as constituents. Thus, as an example, the provided compositions can comprise palmitoyl 16:0.

### B. METHODS

1. Modulating IAP

Disclosed herein are methods for modulating the activity of Intestinal Alkaline Phosphatase (IAP). The disclosed methods include activation of intestinal alkaline phosphatase, as well as inhibition of intestinal alkaline phosphatase.

Disclosed herein are methods for treating various conditions, syndromes, or diseases which are caused by or which result from the lack of or reduced levels of Intestinal Alkaline Phosphatase (IAP). Thus, disclosed is a method for increasing the level of IAP in a subject, comprising administering to a subject in need of treatment an effective amount of one or more compounds disclosed herein. In some aspects, the conditions, syndromes, or diseases involve toxin producing agents. Thus, in some aspects, the conditions, syndromes, or diseases involve LPS from overgrowing bacteria.

Lipopolysaccharide (LPS) is a large molecule consisting of a lipid and a polysaccharide (carbohydrate) joined by a covalent bond. LPS is a major component of the outer membrane of Gram-negative bacteria, contributing greatly to the structural integrity of the bacteria, and protecting the membrane from certain kinds of chemical attack. LPS is an endotoxin, and induces a strong response from normal animal immune systems. The only Gram-positive bacteria that possesses LPS is Listeria monocytogenes, the common infective agent in unpasteurized milk. LPS acts as the prototypical endotoxin, because it binds the CD14/TLR4/MD2 receptor complex, which promotes the secretion of pro-inflammatory cytokines in many cell types, but especially in macrophages. An “LPS challenge” in immunology is the exposing of the subject to an LPS which may act as a toxin. LPS also increases the negative charge of the cell membrane and helps stabilize the overall membrane structure. LPS is additionally an exogenous pyrogen (external fever-inducing compound).

Intestinal alkaline phosphatase (IAP) can detoxify LPS by removing the two phosphate groups found on LPS carbohydrates. This can function as an adaptive mechanism to help the host manage potentially toxic effects of gram-negative bacteria normally found in the small intestine.

However, IAP levels are decreased during malnutrition. As such, the mucosal protection afforded by this enzyme against toxin producing agents, inter alia, bacterial lipopolysaccharide (LPS) is compromised. In addition, growth of luminal microbes which produce other toxins can rapidly occur in the absence of sufficient IAP.

Thus, disclosed herein are methods of treating or preventing bacterial infection resulting from severe malnutrition. The malnutrition can be the result of famine, poverty, digestive disease, malabsorption, depression, anorexia nervosa, bulimia nervosa, fasting, or coma.

Also disclosed herein are methods of treating or preventing bacterial infection in combination with enternal feedings. Tropic enternal feedings are commonly given to small babies, infants, or adult patients that have been treated for long durations, for example, coma, major surgery, or trauma. These feedings are given by tube and contain minimal amounts of food or liquid. These feedings are important so as to prevent the gastrointestinal system from shutting down. Tropic feedings are important in assuring the bowels of these patients continue to function in at least a minimal capacity.

Also disclosed herein are methods of treating or preventing sepsis. Sepsis is a serious medical condition characterized by a whole-body inflammatory state caused by infection. Sepsis is broadly defined as the presence of various pus-forming and other pathogenic organisms, or their toxins, in the blood or tissues. While the term sepsis is frequently used to refer to septicemia (blood poisoning), septicemia is but one type of sepsis. Bacteremia specifically refers to the presence of bacteria in the bloodstream (viremia and fungemia are analogous terms for viruses and fungi).

Also disclosed herein are methods of treating or preventing gastroenteritis. Gastroenteritis refers to inflammation of the gastrointestinal tract, involving both the stomach and the small intestine (see also gastritis and enteritis) and resulting in acute diarrhea. The inflammation is caused most often by infection with certain viruses, bacteria or their toxins, parasites, or adverse reaction to something in the diet or medication. Many different bacteria can cause gastroenteritis, including Salmonella, Shigella, Staphylococcus, Campylobacter jejuni, Clostridium, Escherichia coli, Yersinia, and others. Some sources of the infection are improperly prepared food, reheated meat dishes, seafood, dairy, and bakery products. Each organism causes slightly different symptoms but all result in diarrhea. Colitis, inflammation of the large intestine, may also be present.

Also disclosed herein are methods of treating or preventing bacterial infection coincident with inflammatory bowel disease (IBD). IBD is a group of inflammatory conditions of the large intestine and, in some cases, the small intestine. The main forms of IBD are Crohn's disease and ulcerative colitis (UC). Risk factors are consumption of improperly prepared foods or contaminated water and travel or residence in areas of poor sanitation. The incidence is 1 in 1,000 people.

Another embodiment relates to a method for providing mucosal protection to a subject, comprising administering to a subject in need of treatment an effective amount of one or more compounds disclosed herein.

A further embodiment relates to a method for up regulating the release of intestinal alkaline phosphatase in vivo, in vitro, or ex vivo, comprising administering to a subject in need of treatment an effective amount of one or more compounds disclosed herein.

The luminal phase is the phase in which dietary fats, proteins, and carbohydrates are hydrolyzed and solubilized by secreted digestive enzymes and bile. The mucosal phase relies on the integrity of the brush-border membrane of intestinal epithelial cells to transport digested products from the lumen into the cells. In the postabsorptive phase, reassembled lipids and other key nutrients are transported via lymphatics and portal circulation from epithelial cells to other parts of the body. Perturbation by disease processes in any of these phases frequently results in malabsorption, thus leading to steatorrhea.

Disclosed herein are methods for treating various conditions, syndromes, and disease which are caused by or which result from the poor absorption of fat in the intestine.

Further disclosed is the use of an activator disclosed herein for the use in making a medicament.

Also disclosed is the use of an activator disclosed herein for the use in protecting the intestinal tract of a human or mammal.

Also disclosed is the use of an activator disclosed herein for the use in protecting the intestinal tract of a human or mammal against toxins released by microorganims.

Any of the herein provided methods can further comprise administering to the subject an IAP peptide.

Also provided is a method of enhancing the pyrophosphatase activity of IAP, comprising contacting the IAP with an IAP activator. Although not wishing to be bound by theory, the disclosed IAP activator can facilitate the release of inorganic pyrophosphate (PPᵢ) from the active site, thereby increasing the effective rate of PPᵢ hydrolysis.

The IAP activator of the provided methods can be a macromolecule, such as a polymer. The IAP activator of the provided methods can be a small molecule. Thus, the IAP activator can be a compound disclosed herein. The IAP activator can further be a compound identified as disclosed herein.

The term “effective amount” as used herein means “an amount of one or more compounds, effective at dosages and for periods of time necessary to achieve the desired or therapeutic result.” An effective amount can vary according to factors known in the art, such as the disease state, age, sex, and weight of the human or animal being treated. Although particular dosage regimes can be described in examples herein, a person skilled in the art would appreciated that the dosage regime can be altered to provide optimum therapeutic response. For example, several divided doses can be administered daily or the dose can be proportionally reduced as indicated by the exigencies of the therapeutic situation. In addition, the compositions of this disclosure can be administered as frequently as necessary to achieve a therapeutic amount.

2. Combination Therapies

Provided herein is a composition that comprises an IAP modulator disclosed herein and any known or newly discovered substance that can be administered to the gut mucosa. For example, the provided composition can further comprise one or more of classes of antibiotics (e.g. Aminoglycosides, Cephalosporins, Chloramphenicol, Clindamycin, Erythromycins, Fluoroquinolones, Macrolides, Azolides, Metronidazole, Penicillin's, Tetracycline's, Trimethoprim-sulfamethoxazole, Vancomycin), steroids (e.g. Andranes (e.g. Testosterone), Cholestanes (e.g. Cholesterol), Cholic acids (e.g. Cholic acid), Corticosteroids (e.g. Dexamethasone), Estraenes (e.g. Estradiol), Pregnanes (e.g. Progesterone), narcotic and non-narcotic analgesics (e.g. Morphine, Codeine, Heroin, Hydromorphone, Levorphanol, Meperidine, Methadone, Oxydone, Propoxyphene, Fentanyl, Methadone, Naloxone, Buprenorphine, Butorphanol, Nalbuphine, Pentazocine), anti-inflammatory agents (e.g. Alclofenac; Alclometasone Dipropionate; Algestone Acetonide; alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Decanoate; Deflazacort; Delatestryl; Depo-Testosterone; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Mesterolone; Methandrostenolone; Methenolone; Methenolone Acetate; Methylprednisolone Suleptanate; Morniflumate; Nabumetone; Nandrolone; Naproxen; Naproxen Sodium; Naproxol; Nimazone; Olsalazine Sodium; Orgotein; Orpanoxin; Oxandrolane; Oxaprozin; Oxyphenbutazone; Oxymetholone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Stanozolol; Sudoxicam; Sulindac; Suprofen; Talmetacin; Talniflumate; Talosalate; Tebufelone; Tenidap; Tenidap Sodium; Tenoxicam; Tesicam; Tesimide; Testosterone; Testosterone Blends; Tetrydamine; Tiopinac; Tixocortol Pivalate; Tolmetin; Tolmetin Sodium; Triclonide; Triflumidate; Zidometacin; Zomepirac Sodium), or anti-histaminic agents (e.g. Ethanolamines (like diphenhydrmine carbinoxamine), Ethylenediamine (like tripelennamine pyrilamine), Alkylamine (like chlorpheniramine, dexchlorpheniramine, brompheniramine, triprolidine), other anti-histamines like astemizole, loratadine, fexofenadine, Bropheniramine, Clemastine, Acetaminophen, Pseudoephedrine, Triprolidine).

3. Administration

The disclosed compounds and compositions can be administered in any suitable manner. The manner of administration can be chosen based on, for example, whether local or systemic treatment is desired, and on the area to be treated. For example, the compositions can be administered orally, parenterally (e.g., intravenous, subcutaneous, intraperitoneal, or intramuscular injection), by inhalation, extracorporeally, topically (including transdermally, ophthalmically, vaginally, rectally, intranasally) or the like.

As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The exact amount of the compositions required can vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. Thus, effective dosages and schedules for administering the compositions can be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage can vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products.

For example, a typical daily dosage of the IAP modulators disclosed herein used alone might range from about 1 μg/kg to up to 100 mg/kg of body weight or more per day, depending on the factors mentioned above.

Following administration of a disclosed composition for treating, inhibiting, or preventing a gut mucosal infection, the efficacy of the therapeutic IAP modulator can be assessed in various ways well known to the skilled practitioner.

The IAP modulators disclosed herein can be administered prophylactically to patients or subjects who are at risk for gut mucosal infections or who have been newly diagnosed with a gut mucosal infection.

The disclosed compositions and methods can also be used for example as tools to isolate and test new drug candidates for a variety of gastrointestinal related diseases.

4. Screening Method

Disclosed herein is a method of screening compounds to identify an IAP activator. In general, the method involves detecting dephosphorylation of an AP substrate. For example, the method can be a chemiluminescent method of detecting substrate dephosphorylation.

i. Substrates

The AP substrate can be, for example, a 1,2-dioxetane compound. 1,2-dioxetane enzyme substrates have been well established as highly efficient chemiluminescent reporter molecules for use in enzyme immunoassays of a wide variety of types. These assays provide an alternative to conventional assays that rely on radioisotopes, fluorophores, complicated color shifting, secondary reactions and the like. Dioxetanes developed for this purpose include those disclosed in U.S. Pat. No. 4,978,614 and U.S. Pat. No. 5,112,960. U.S. Pat. No. 4,978,614 discloses, among others, 3-(2′-spiroadamantane)-4-methoxy-4-(3″-phosphoryloxy)phenyl-1,2-dioxetane, which commercially available under the trade name AMPPD. U.S. Pat. No. 5,112,960, discloses dioxetane compounds, wherein the adamantyl stabilizing ring is substituted, at either bridgehead position, with a variety of substituents, including hydroxy, halogen, and the like, which convert the otherwise static or passive adamantyl stabilizing group into an active group involved in the kinetics of decomposition of the dioxetane ring. CSPD is a spiroadamantyl dioxetane phenyl phosphate with a chlorine substituent on the adamantyl group.

The AP substrate can be CSPD® (Disodium 3-(4-methoxyspiro{1,2-dioxetane-3,2′-(5′-chloro)tricyclo[3.3.1.13,7]decan}-4-yl)phenyl phosphate) or CDP-Star® (Disodium 2-chloro-5-(4-methoxyspiro {1,2-dioxetane-3,2′-(5′-chloro)-ricyclo[3.3.1.13,7]decan}-4-yl)-1-phenyl phosphate) substrates (Applied Biosystems, Bedford, Mass.). CSPD® and CDP-Star® substrates produce a luminescent signal when acted upon by AP, which dephosphorylates the substrates and yields anions that ultimately decompose, resulting in light emission. Light production resulting from chemical decomposition exhibits an initial delay followed by a persistent glow that lasts as long as free substrate is available. The glow signal can endure for hours or even days if signal intensity is low; signals with very high intensities may only last for a few hours. With CSPD® substrate, peak light emission is obtained in 10-20 min in solution assays, or in about four hours on a nylon membrane; CDP-Star® substrate exhibits solution kinetics similar to CSPD® substrate, but reaches peak light emission on a membrane in only 1-2 hours. Despite these long times to peak signal intensity, however, X-ray film exposure usually only requires 15 sec to 15 min with standard X-ray film. Both substrates provide high detection sensitivity, fast X-ray film exposure, superior band resolution, and glow light emission kinetics, enabling acquisition of multiple film exposures and use of luminometers without automatic reagent injectors. CDP-Star® substrate exhibits a brighter signal (5-10-fold) and a faster time to peak light emission on membranes, making CDP-Star® substrate the preferred choice when imaging membranes on digital signal acquisition systems.

AP substrates can be in an alkaline hydrophobic environment. Thus, substrate formulations can be in an alkaline buffer solution.

The AP substrates can be used in conjunction with enhancement agents, which include natural and synthetic water-soluble macromolecules, which are disclosed in detail in U.S. Pat. No. 5,145,772. Example enhancement agents include water-soluble polymeric quaternary ammonium salts, such as poly(vinylbenzyltrimethylammonium chloride) (TMQ), poly(vinylbenzyltributylammonium chloride) (TBQ) and poly(vinylbenzyldimethylbenzylammonium chloride) (BDMQ). These enhancement agents improve the chemiluminescent signal of the dioxetane reporter molecules, by providing a hydrophobic environment in which the dioxetane is sequestered. Water, an unavoidable aspect of most assays, due to the use of body fluids, is a natural “quencher” of the dioxetane chemiluminescence. The enhancement molecules can exclude water from the microenvironment in which the dioxetane molecules, or at least the excited state emitter species reside, resulting in enhanced chemiluminescence. Other effects associated with the enhancer-dioxetane interaction could also contribute to the chemiluminescence enhancement.

Additional advantages can be secured by the use of selected membranes, including nylon membranes and treated nitrocellulose, providing a similarly hydrophobic surface for membrane-based assays, and other membranes coated with the enhancer-type polymers described.

The disclosed reaction is 2, 3, or 4 orders of magnitude more sensitive than previously utilized colorimetric assays, a quality that allowed a decrease the concentration of AIP, but more importantly the ability to screen in the presence of a 5-fold, 6-fold, 7-fold, 8-fold, 9-fold, or 10-fold lower concentration of diethanolamine (DEA). The luminescence signal can be linear over a 2-, 3-, or 4-orders-of-magnitude range of AIP concentrations.

The disclosed luminescent assay can be further optimized to ensure its maximum sensitivity to compounds activating AIP. For example, DEA buffer can be replaced with CAPS that does not contain any alcohol phosphoacceptor. This assay can provide a more accurate measure of phosphatase activity, as opposed to transphosphorylation activity that might be more relevant to in vivo conditions.

The concentration of CDP-star® can be fixed at 25 uM (˜Kₘ) to provide enough sensitivity even for compounds competitive with the CDP-star® substrate.

Half-maximal activation can correspond to 127 mM DEA. Maximal activation can result in 9.4-fold higher activity than in the absence of DEA. 600 mM DEA (pH 9.8) (e.g., in 2% DMSO) can be chosen as a positive control for AIP activation screening. The performance of the assay can be tested in the presence and absence of DEA.

Also disclosed is a method of screening for modulators of AIP using a colorimetric assay system, wherein the colorimetric assay system uses a phosphate-based substrate. The screening can be performed in the presence of saturating concentrations of diethanolamine. The phosphate can be p-nitrophenyl phosphate or dioxetane-phosphate.

Also disclosed is a method of identifying compounds which are capable of activating AIP activity in animals comprising the steps of selecting compounds to be screened for activating AIP; determining the activity of the AIP in an in vitro assay in the presence and the absence of each compound to be screened; and comparing the activity of the AIP in the presence and the absence of the compounds to be screened to identify compounds which are capable of activating AIP activity in animals.

In this method, the compounds can be capable of activating the AIP's pyrophosphatase activity. The compounds can be further administered alone for the treatment of osteoporosis in animals. Alternatively, the compounds can be administered with recombinant AIP for the treatment of osteoporosis in animals. Similarly, the compounds can be administered alone or with recombinant AIP to reduce the effects of hypophosphatasia in animals. The compounds can allow tapering of administration of recombinant AIP. The compounds can serve as a means of upregulating the AIP activity in conjunction with enzyme replacement therapy for treatment of heritable bone disorders. Alternatively, the compounds can serve as a means of upregulating the AIP activity without using enzyme replacement therapy in animals suffering from osteoporosis. The compounds can also serve as a means of inducing higher bone mineral densities by upregulating AIP activity or as a means of inducing higher bone mineral densities by reducing calcification inhibitors.

ii. Compounds

Libraries of compounds, such as Molecular Libraries Screening Center Network (MLSCN) compounds, can be screened using the disclosed assay in search of compounds that are potent activators of IAP. In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetic libraries are produced, if desired, according to methods known in the art, e.g., by standard extraction and fractionation methods. Furthermore, if desired, any library or compound is readily modified using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect on the activity of AIP should be employed whenever possible.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that stimulates or inhibits AIP. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions in which it is desirable to regulate or mimic activity of AIP.

### C. METHODS OF MAKING THE COMPOSITIONS

The compositions disclosed herein and the compositions necessary to perform the disclosed methods can be made using any method known to those of skill in the art for that particular reagent or compound unless otherwise specifically noted.

### D. DEFINITIONS

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

It must be noted that as used herein and in the appended claims, the singular forms “a,” “an,” and “the” include plural reference unless the context clearly dictates otherwise. Thus, for example, reference to “a composition” includes a plurality of such compositions, reference to “the composition” is a reference to one or more compositions and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

The following chemical hierarchy is used throughout the specification to describe and enable the scope of the disclosed compounds and to particularly point out and distinctly claim the units which comprise the disclosed compounds, however, unless otherwise specifically defined, the terms used herein are the same as those of the artisan of ordinary skill. The term “hydrocarbyl” stands for any carbon atom-based unit (organic molecule), said units optionally containing one or more organic functional group, including inorganic atom comprising salts, inter alia, carboxylate salts, quaternary ammonium salts. Within the broad meaning of the term “hydrocarbyl” are the classes “acyclic hydrocarbyl” and “cyclic hydrocarbyl” which terms are used to divide hydrocarbyl units into cyclic and non-cyclic classes.

As it relates to the following definitions, “cyclic hydrocarbyl” units can comprise only carbon atoms in the ring (carbocyclic and aryl rings) or can comprise one or more heteroatoms in the ring (heterocyclic and heteroaryl). For “carbocyclic” rings the lowest number of carbon atoms in a ring are 3 carbon atoms; cyclopropyl. For “aryl” rings the lowest number of carbon atoms in a ring are 6 carbon atoms; phenyl. For “heterocyclic” rings the lowest number of carbon atoms in a ring is 1 carbon atom; diazirinyl. Ethylene oxide comprises 2 carbon atoms and is a C₂ heterocycle. For “heteroaryl” rings the lowest number of carbon atoms in a ring is 1 carbon atom; 1,2,3,4-tetrazolyl. The following is a non-limiting description of the terms “acyclic hydrocarbyl” and “cyclic hydrocarbyl” as used herein.

A. Substituted and unsubstituted acyclic hydrocarbyl:

As used herein, the term “substituted and unsubstituted acyclic hydrocarbyl” encompasses 3 categories of units:

1) linear or branched alkyl, non-limiting examples of which include, methyl (C₁), ethyl (C₂), n-propyl (C₃), iso-propyl (C₃), n-butyl (C₄), sec-butyl (C₄), iso-butyl (C₄), tert-butyl (C₄), and the like; substituted linear or branched alkyl, non-limiting examples of which includes, hydroxymethyl (C₁), chloromethyl (C₁), trifluoromethyl (C₁), aminomethyl (C₁), 1-chloroethyl (C₂), 2-hydroxyethyl (C₂), 1,2-difluoroethyl (C₂), 3-carboxypropyl (C₃), and the like.

2) linear or branched alkenyl, non-limiting examples of which include, ethenyl (C₂), 3-propenyl (C₃), 1-propenyl (also 2-methylethenyl) (C₃), isopropenyl (also 2-methylethen-2-yl) (C₃), buten-4-yl (C₄), and the like; substituted linear or branched alkenyl, non-limiting examples of which include, 2-chloroethenyl (also 2-chlorovinyl) (C₂), 4-hydroxybuten-1-yl (C₄), 7-hydroxy-7-methyloct-4-en-2-yl (C₉), 7-hydroxy-7-methyloct-3,5-dien-2-yl (C₉), and the like.

3) linear or branched alkynyl, non-limiting examples of which include, ethynyl (C₂), prop-2-ynyl (also propargyl) (C₃), propyn-1-yl (C₃), and 2-methyl-hex-4-yn-1-yl (C₇); substituted linear or branched alkynyl, non-limiting examples of which include, 5-hydroxy-5-methylhex-3-ynyl (C₇), 6-hydroxy-6-methylhept-3-yn-2-yl (C₈), 5-hydroxy-5-ethylhept-3-ynyl (C₉), and the like.

B. Substituted and unsubstituted cyclic hydrocarbyl:

As used herein, the term “substituted and unsubstituted cyclic hydrocarbyl” encompasses 5 categories of units:

1) The term “carbocyclic” is defined herein as “encompassing rings comprising from 3 to 20 carbon atoms, wherein the atoms which comprise said rings are limited to carbon atoms, and further each ring can be independently substituted with one or more moieties capable of replacing one or more hydrogen atoms.” The following are non-limiting examples of “substituted and unsubstituted carbocyclic rings” which encompass the following categories of units:

i) carbocyclic rings having a single substituted or unsubstituted hydrocarbon ring, non-limiting examples of which include, cyclopropyl (C₃), 2-methyl-cyclopropyl (C₃), cyclopropenyl (C₃), cyclobutyl (C₄), 2,3-dihydroxycyclobutyl (C₄), cyclobutenyl (C₄), cyclopentyl (C₅), cyclopentenyl (C₅), cyclopentadienyl (C₅), cyclohexyl (C₆), cyclohexenyl (C₆), cycloheptyl (C₇), cyclooctanyl (C₈), decalinyl (C₁₀), 2,5-dimethylcyclopentyl (C₅), 3,5-dichlorocyclohexyl (C₆), 4-hydroxycyclohexyl (C₆), and 3,3,5-trimethylcyclohex-1-yl (C₆).

ii) carbocyclic rings having two or more substituted or unsubstituted fused hydrocarbon rings, non-limiting examples of which include, octahydropentalenyl (C₈), octahydro-1H-indenyl (C₉), 3a,4,5,6,7,7a-hexahydro-3H-inden-4-yl (C₉), decahydroazulenyl (C₁₀).

iii) carbocyclic rings which are substituted or unsubstituted bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3,3]undecanyl.

2) The term “aryl” is defined herein as “units encompassing at least one phenyl or naphthyl ring and wherein there are no heteroaryl or heterocyclic rings fused to the phenyl or naphthyl ring and further each ring can be independently substituted with one or more moieties capable of replacing one or more hydrogen atoms.” The following are non-limiting examples of “substituted and unsubstituted aryl rings” which encompass the following categories of units:

i) C₆ or C₁₀ substituted or unsubstituted aryl rings; phenyl and naphthyl rings whether substituted or unsubstituted, non-limiting examples of which include, phenyl (C₆), naphthylen-1-yl (C₁₀), naphthylen-2-yl (C₁₀), 4-fluorophenyl (C₆), 2-hydroxyphenyl (C₆), 3-methylphenyl (C₆), 2-amino-4-fluorophenyl (C₆), 2-(N,N-diethylamino)phenyl (C₆), 2-cyanophenyl (C₆), 2,6-di-tert-butylphenyl (C₆), 3-methoxyphenyl (C₆), 8-hydroxynaphthylen-2-yl (C₁₀), 4,5-dimethoxynaphthylen-1-yl (C₁₀), and 6-cyano-naphthylen-1-yl (C₁₀).

ii) C₆ or C₁₀ aryl rings fused with 1 or 2 saturated rings non-limiting examples of which include, bicyclo[4.2.0]octa-1,3,5-trienyl (C₈), and indanyl (C₉).

3) The terms “heterocyclic” and/or “heterocycle” are defined herein as “units comprising one or more rings having from 3 to 20 atoms wherein at least one atom in at least one ring is a heteroatom chosen from nitrogen (N), oxygen (O), or sulfur (S), or mixtures of N, O, and S, and wherein further the ring which comprises the heteroatom is also not an aromatic ring.” The following are non-limiting examples of “substituted and unsubstituted heterocyclic rings” which encompass the following categories of units:

i) heterocyclic units having a single ring containing one or more heteroatoms, non-limiting examples of which include, diazirinyl (C₁), aziridinyl (C₂), urazolyl (C₂), azetidinyl (C₃), pyrazolidinyl (C₃), imidazolidinyl (C₃), oxazolidinyl (C₃), isoxazolinyl (C₃), isoxazolyl (C₃), thiazolidinyl (C₃), isothiazolyl (C₃), isothiazolinyl (C₃), oxathiazolidinonyl (C₃), oxazolidinonyl (C₃), hydantoinyl (C₃), tetrahydrofuranyl (C₄), pyrrolidinyl (C₄), morpholinyl (C₄), piperazinyl (C₄), piperidinyl (C₄), dihydropyranyl (C₅), tetrahydropyranyl (C₅), piperidin-2-onyl (valerolactam) (C₅), 2,3,4,5-tetrahydro-1H-azepinyl (C₆), 2,3-dihydro-1H-indole (C₈), and 1,2,3,4-tetrahydro-quinoline (C₉).

ii) heterocyclic units having 2 or more rings one of which is a heterocyclic ring, non-limiting examples of which include hexahydro-1H-pyrrolizinyl (C₇), 3a,4,5,6,7,7a-hexahydro-1H-benzo[d]imidazolyl (C₇), 3a,4,5,6,7,7a-hexahydro-1H-indolyl (C₈), 1,2,3,4-tetrahydroquinolinyl (C₉), and decahydro-1H-cycloocta[b]pyrrolyl (C₁₀).

4) The term “heteroaryl” is defined herein as “encompassing one or more rings comprising from 5 to 20 atoms wherein at least one atom in at least one ring is a heteroatom chosen from nitrogen (N), oxygen (O), or sulfur (S), or mixtures of N, O, and S, and wherein further at least one of the rings which comprises a heteroatom is an aromatic ring.” The following are non-limiting examples of “substituted and unsubstituted heterocyclic rings” which encompass the following categories of units:

i) heteroaryl rings containing a single ring, non-limiting examples of which include, 1,2,3,4-tetrazolyl (C₁), [1,2,3]triazolyl (C₂), [1,2,4]triazolyl (C₂), triazinyl (C₃), thiazolyl (C₃), 1H-imidazolyl (C₃), oxazolyl (C₃), furanyl (C₄), thiopheneyl (C₄), pyrimidinyl (C₄), 2-phenylpyrimidinyl (C₄), pyridinyl (C₅), 3-methylpyridinyl (C₅), and 4-dimethylaminopyridinyl (C₅)

ii) heteroaryl rings containing 2 or more fused rings one of which is a heteroaryl ring, non-limiting examples of which include: 7H-purinyl (C₅), 9H-purinyl (C₅), 6-amino-9H-purinyl (C₅), 5H-pyrrolo[3,2-d]pyrimidinyl (C₆), 7H-pyrrolo[2,3-d]pyrimidinyl (C₆), pyrido[2,3-d]pyrimidinyl (C₇), 2-phenylbenzo[d]thiazolyl (C₇), 1H-indolyl (C₈), 4,5,6,7-tetrahydro-1-H-indolyl (C₈), quinoxalinyl (C₈), 5-methylquinoxalinyl (C₈), quinazolinyl (C₈), quinolinyl (C₉), 8-hydroxy-quinolinyl (C₉), and isoquinolinyl (C₉).

5) C₁-C₆ tethered cyclic hydrocarbyl units (whether carbocyclic units, C₆ or C₁₀ aryl units, heterocyclic units, or heteroaryl units) which connected to another moiety, unit, or core of the molecule by way of a C₁-C₆ alkylene unit. Non-limiting examples of tethered cyclic hydrocarbyl units include benzyl C₁-(C₆) having the formula:

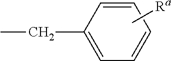

wherein R^{a} is optionally one or more independently chosen substitutions for hydrogen. Further examples include other aryl units, inter alia, (2-hydroxyphenyl)hexyl C₆-(C₆); naphthalen-2-ylmethyl C₁-(C₁₀), 4-fluorobenzyl C₁-(C₆), 2-(3-hydroxy-phenyl)ethyl C₂-(C₆), as well as substituted and unsubstituted C₃-C₁₀ alkylenecarbocyclic units, for example, cyclopropylmethyl C₁-(C₃), cyclopentylethyl C₂-(C₅), cyclohexylmethyl C₁-(C₆). Included within this category are substituted and unsubstituted C₁-C₁₀ alkylene-heteroaryl units, for example a 2-picolyl C₁-(C₆) unit having the formula:

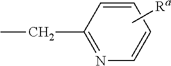

wherein R^{a} is the same as defined above. In addition, C₁-C₁₂ tethered cyclic hydrocarbyl units include C₁-C₁₀ alkyleneheterocyclic units and alkylene-heteroaryl units, non-limiting examples of which include, aziridinylmethyl C₁-(C₂) and oxazol-2-ylmethyl C₁-(C₃).

As used herein, carbocyclic rings are from C₃ to C₂₀; aryl rings are C₆ or C₁₀; heterocyclic rings are from C₁ to C₉; and heteroaryl rings are from C₁ to C₉.

As used herein, fused ring units, as well as spirocyclic rings, bicyclic rings and the like, which comprise a single heteroatom are characterized and referred to herein as being encompassed by the cyclic family corresponding to the heteroatom containing ring, although the artisan can have alternative characterizations. For example, 1,2,3,4-tetrahydroquinoline having the formula:

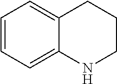

is considered a heterocyclic unit. 6,7-Dihydro-5H-cyclopentapyrimidine having the formula:

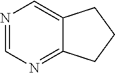

is considered a heteroaryl unit. When a fused ring unit contains heteroatoms in both a saturated ring (heterocyclic ring) and an aryl ring (heteroaryl ring), the aryl ring can predominate and determine the type of category to which the ring is assigned herein. For example, 1,2,3,4-tetrahydro-[1,8]naphthyridine having the formula:

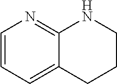

is considered a heteroaryl unit.

The term “substituted” is used throughout the specification. The term “substituted” is applied to the units described herein as “substituted unit or moiety is a hydrocarbyl unit or moiety, whether acyclic or cyclic, which has one or more hydrogen atoms replaced by a substituent or several substituents as defined herein below.” The units, when substituting for hydrogen atoms are capable of replacing one hydrogen atom, two hydrogen atoms, or three hydrogen atoms of a hydrocarbyl moiety at a time. In addition, these substituents can replace two hydrogen atoms on two adjacent carbons to form said substituent, new moiety, or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. Three hydrogen replacement includes cyano, and the like. The term substituted is used throughout the present specification to indicate that a hydrocarbyl moiety, inter alia, aromatic ring, alkyl chain; can have one or more of the hydrogen atoms replaced by a substituent. When a moiety is described as “substituted” any number of the hydrogen atoms can be replaced. For example, 4-hydroxyphenyl is a “substituted aromatic carbocyclic ring (aryl ring)”, (N,N-dimethyl-5-amino)octanyl is a “substituted C₈ linear alkyl unit, 3-guanidinopropyl is a “substituted C₃ linear alkyl unit,” and 2-carboxypyridinyl is a “substituted heteroaryl unit.”

The following are non-limiting examples of units which can substitute for hydrogen atoms on a carbocyclic, aryl, heterocyclic, or heteroaryl unit:
i) C₁-C₁₂ linear, branched, or cyclic alkyl, alkenyl, and alkynyl; methyl (C₁), ethyl (C₂), ethenyl (C₂), ethynyl (C₂), n-propyl (C₃), iso-propyl (C₃), cyclopropyl (C₃), 3-propenyl (C₃), 1-propenyl (also 2-methylethenyl) (C₃), isopropenyl (also 2-methylethen-2-yl) (C₃), prop-2-ynyl (also propargyl) (C₃), propyn-1-yl (C₃), n-butyl (C₄), sec-butyl (C₄), iso-butyl (C₄), tert-butyl (C₄), cyclobutyl (C₄), buten-4-yl (C₄), cyclopentyl (C₅), cyclohexyl (C₆);
ii) substituted or unsubstituted C₆ or C₁₀ aryl; for example, phenyl, naphthyl (also referred to herein as naphthylen-1-yl (C₁₀) or naphthylen-2-yl (C₁₀));
iii) substituted or unsubstituted C₆ or C₁₀ alkylenearyl; for example, benzyl, 2-phenylethyl, naphthylen-2-ylmethyl;
iv) substituted or unsubstituted C₁-C₉ heterocyclic rings; as described herein below;
v) substituted or unsubstituted C₁-C₉ heteroaryl rings; as described herein below;
vi) —(CR^{102a}R^{102b})_{z}OR¹⁰¹; for example, —OH, —CH₂OH, —OCH₃, —CH₂OCH₃, —OCH₂CH₃, —CH₂OCH₂CH₃, —OCH₂CH₂CH₃, and —CH₂OCH₂CH₂CH₃;
vii) —(CR^{102a}R^{102b})_{z}C(O)R¹⁰¹; for example, —COCH₃, —CH₂COCH₃, —OCH₂CH₃, —CH₂COCH₂CH₃, —COCH₂CH₂CH₃, and —CH₂COCH₂CH₂CH₃;
viii) —(CR^{102a}R^{102b})_{z}C(O)OR¹⁰¹; for example, —CO₂CH₃, —CH₂CO₂CH₃, —CO₂CH₂CH₃, —CH₂CO₂CH₂CH₃, —CO₂CH₂CH₂CH₃, and —CH₂CO₂CH₂CH₂CH₃;
ix) —(CR^{102a}R^{102b})_{z}C(O)N(R¹⁰¹)₂; for example, —CONH₂, —CH₂CONH₂, —CONHCH₃, —CH₂CONHCH₃, —CON(CH₃)₂, and —CH₂CON(CH₃)₂;
x) —(CR^{102a}R^{102b})_{z}N(R¹⁰¹)₂; for example, —NH₂, —CH₂NH₂, —NHCH₃, —CH₂NHCH₃, —N(CH₃)₂, and —CH₂N(CH₃)₂;
xi) halogen; —F, —Cl, —Br, and —I;
xii) —(CR^{102a}R^{102b})_{z}CN;
xiii) —(CR^{102a}R^{102b})_{z}NO₂;
xiv) —CHⱼXₖ; wherein X is halogen, the index j is an integer from 0 to 2, j+k 3; for example, —CH₂F, —CHF₂, —CF₃, —CCl₃, or —CBr₃;
xv) —(CR^{102a}R^{102b})_{z}SR¹⁰¹; —SH, —CH₂SH, —SCH₃, —CH₂SCH₃, —SC₆H₅, and —CH₂SC₆H₅;
xvi) —(CR^{102a}R^{102b})_{z}SO₂R¹⁰¹; for example, —SO₂H, —CH₂SO₂H, —SO₂CH₃, —CH₂SO₂CH₃, —SO₂C₆H₅, and —CH₂SO₂C₆H₅; and
xvii) —(CR^{102a}R^{102b})_{z}SO₃R¹⁰¹; for example, —SO₃H, —CH₂SO₃H, —SO₃CH₃, —CH₂SO₃CH₃, —SO₃C₆H₅, and —CH₂SO₃C₆H₅;

wherein each R¹⁰¹ is independently hydrogen, substituted or unsubstituted C₁-C₄ linear, branched, or cyclic alkyl, phenyl, benzyl, heterocyclic, or heteroaryl; or two R¹⁰¹ units can be taken together to form a ring comprising 3-7 atoms; R^{102a} and R^{102b} are each independently hydrogen or C₁-C₄ linear or branched alkyl; the index z is from 0 to 4

For the purposes of the present disclosure the terms “compound,” “analog,” and “composition of matter” stand equally well for the Intestinal Alkaline Phosphatase (AIP) activators or inhibitors described herein, including all enantiomeric forms, diastereomeric forms, salts, and the like, and the terms “compound,” “analog,” and “composition of matter” are used interchangeably throughout the present specification.

The compounds disclosed herein include all salt forms, for example, salts of both basic groups, inter alia, amines, as well as salts of acidic groups, inter alia, carboxylic acids. The following are non-limiting examples of anions that can form salts with basic groups: chloride, bromide, iodide, sulfate, bisulfate, carbonate, bicarbonate, phosphate, formate, acetate, propionate, butyrate, pyruvate, lactate, oxalate, malonate, maleate, succinate, tartrate, fumarate, citrate, and the like. The following are non-limiting examples of cations that can form salts of acidic groups: sodium, lithium, potassium, calcium, magnesium, bismuth, and the like.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Akp6 is Upregulated in Intestines of Akp3 Knockout Mice

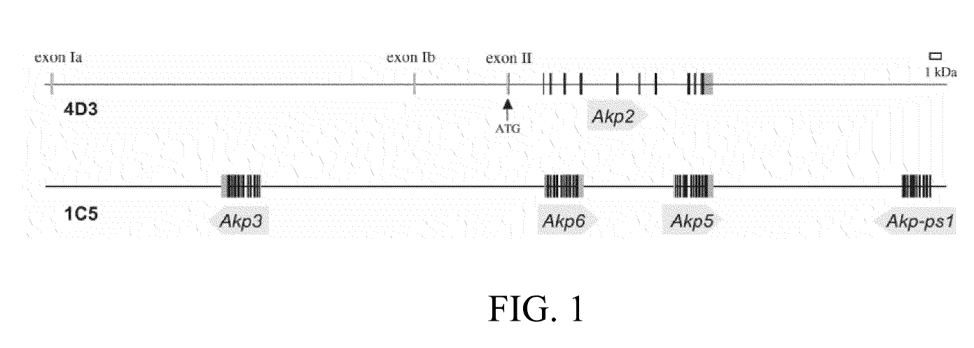

The epithelium of the mouse small intestine expresses two intestine specific AP genes, Akp3 and Akp6, and low levels of Akp5, which is not intestine specific (Narisawa, et al, 2007). The genomic organization of these genes are shown in FIG. 1. AP proteins encoded by Akp3, Akp5 and Akp6 were designated duodenal IAP or dIAP, embryonic AP or EAP and global IAP or gIAP, respectively. The peptide sequences of dIAP and gIAP have 87% homology, while EAP shows slightly lower sequence similarity to the others. Kinetics studies with recombinant proteins encoded by the three genes indicated that dIAP had the highest Km value and appeared to be the most efficient enzyme at least in vitro using the artificial substrate, p-nitrophenyl phosphate (pNPP), at alkaline pH (Table 2).

TABLE 2
Kinetic parameters of recombinant mouse dIAP, gIAP,
and EAP using p-NPP as a substrate at pH 9.8.

| Isozyme | k_{cat}, s⁻¹ | Kₘ, mM | k_{cat}/Kₘ, s⁻¹ · M⁻¹ |
|---|---|---|---|
| dIAP | 339 ± 13 | 1.1 ± 0.34 | 0.3 |
| gIAP | 50 ± 1.4 | 0.79 ± 0.17 | 0.074 |
| EAP | 8.4 ± 1.1 | 0.14 ± 0.03 | 0.062 |

Values are means ± SD. k_{cat}, catalytic rate constant

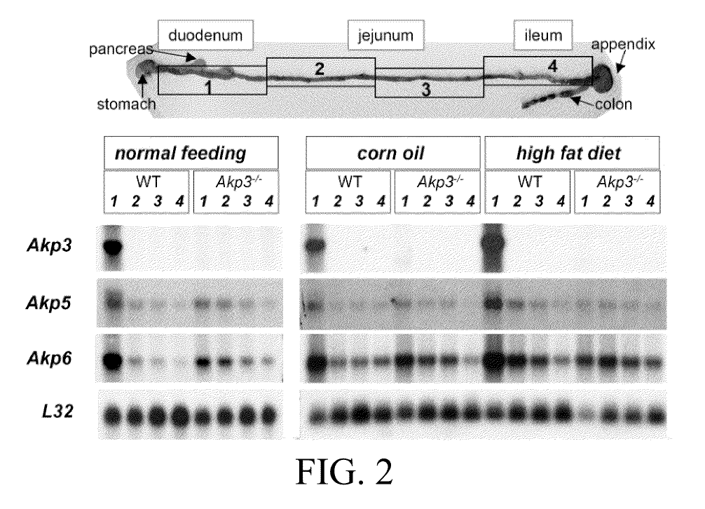

Northern and Western blot analyses show that Akp3 (dIAP) is strictly expressed in the duodenum, while Akp6 (gIAP) is expressed in the entire small intestine. Akp5 (EAP), originally identified in pre-implantation embryos and testis (Hahnel, et al., 1990, Narisawa, et al., 1992), is also expressed at lower levels in the entire small intestine. Northern blots using gene specific probes (FIG. 2) show that Akp6 expression in the distal small intestine is upregulated in Akp3^{-/-} mice, and that both wild-type and Akp3 null mice forced-fed with corn oil or fed a high fat diet show increased levels of Akp6 mRNA in the jejunum and ileum.

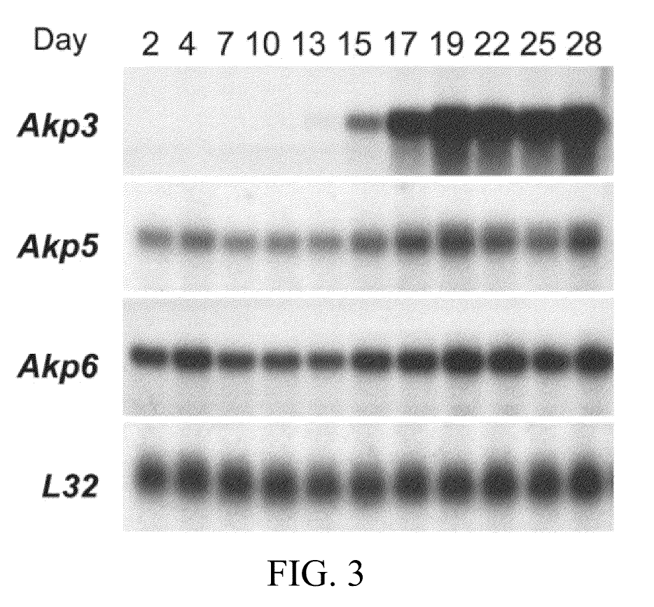

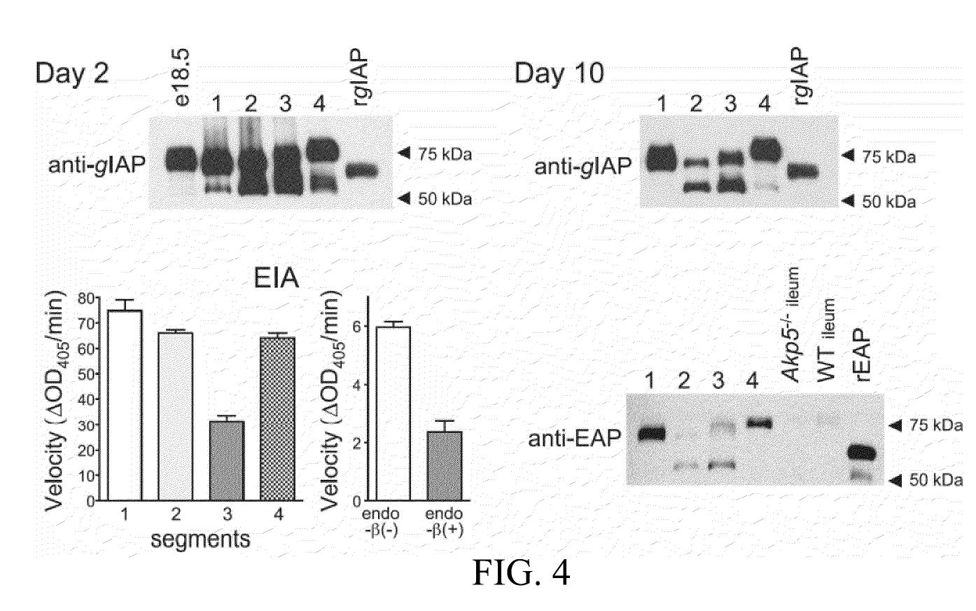

Akp3 expression begins at postnatal day ˜15, while Akp5 and Akp6 are expressed in all postnatal stages as shown in Northern blots (FIG. 3). Antibodies were raised against the specific peptides deduced from Akp3, Akp5 and Akp6 sequences. Western analysis identified dIAP protein in the duodenum samples as a wide 80-75 kDa band, a pattern typical of a highly glycosylated protein (SDS-PAGE under reducing conditions). gIAP was detected in the entire small intestine and showed a molecular weight of ˜75 kDa. Interestingly pre-weaning stage intestines showed at least two different molecular sizes for gIAP: ˜75 kDa and ˜55 kDa. The smaller species corresponds to the predicted molecular mass of an unmodified GPI-anchored gIAP polypeptide (54,526 Da) (Day 2 and Day 10 in FIG. 4). The larger band observed in intestinal Segment 4 (distal 25%) appears to be the same size as gIAP detected in adult gut. To examine the catalytic properties of these gIAP isoforms, four intestinal segments (25% each from proximal to distal) from 2-day-old WT mice were homogenized in Tris buffer (pH 8.9) containing 0.1% Triton X-100 and extracted with n-butyl alcohol. Extracts (1.5 mg/ml protein concentration) were incubated in 96-well plates coated with the anti-gIAP antibody (# 3766). Enzymatic activity of specifically bound gIAP protein was measured with serial concentrations of substrate (20, 10, 5, 2.5, 1.25, and 0 mM pNPP). Intestinal Segment 3 (corresponding to jejunum) showed lower Kₘ values (0.77±0.20 mM) than Segment 1 (duodenum; 0.86±0.13 mM) or Segment 4 (ileum; 1.00±0.44 mM). Enzymatic activity was also lower in Segment 3 (left bottom, FIG. 4). To assess whether the change in molecular mass was associated with N-linked glycosylation particularly by polylactosamines (Fukuda MN, 1992), butanol extracts of Segment 1 from 2-day-old mice were bound onto anti-gIAP-antibody coated 96-well plates. After washing, wells were treated with 0.003 units of endo-β-galactosidase for 16 hours. Endo-β-galactosidase specifically cleaves β-galactosidic linkage in polylactosamines. This enzymatic treatment reduced gIAP activity to levels comparable to those present in Segment 3, indicating that changes in polylactosamines modulate catalytic properties of gIAP. A similar change was observed for EAP (right bottom in FIG. 4). These data indicate that enterocytes in a part of jejunum of the neonatal gut are unable to fully glycosylate these glycoproteins, consistent with the developmental expression of galactosyl-transferases in the postnatal gut (Ozaki, et al., 1989). Active gIAP enzyme in proximal and distal intestine can be advantageous to detoxify pathogenic bacteria from the mouth and large intestines in neonatal animals. Also the existence of a region of intestinal mucosa lacking any active IAP at early postnatal stages can allow the immune system to develop tolerance to certain bacteria with intact/phosphorylated LPS and to establish a symbiotic/commensal relationship with intestinal flora in future adult stages.

### 2. Example 2

### Intestinal Alkaline Phosphatase is a Gut Mucosal Defense Factor Maintained by Enteral Nutrition

i. Effect of IAP Expression on LPS Signaling in Cells Over Expressing Recombinant IAP

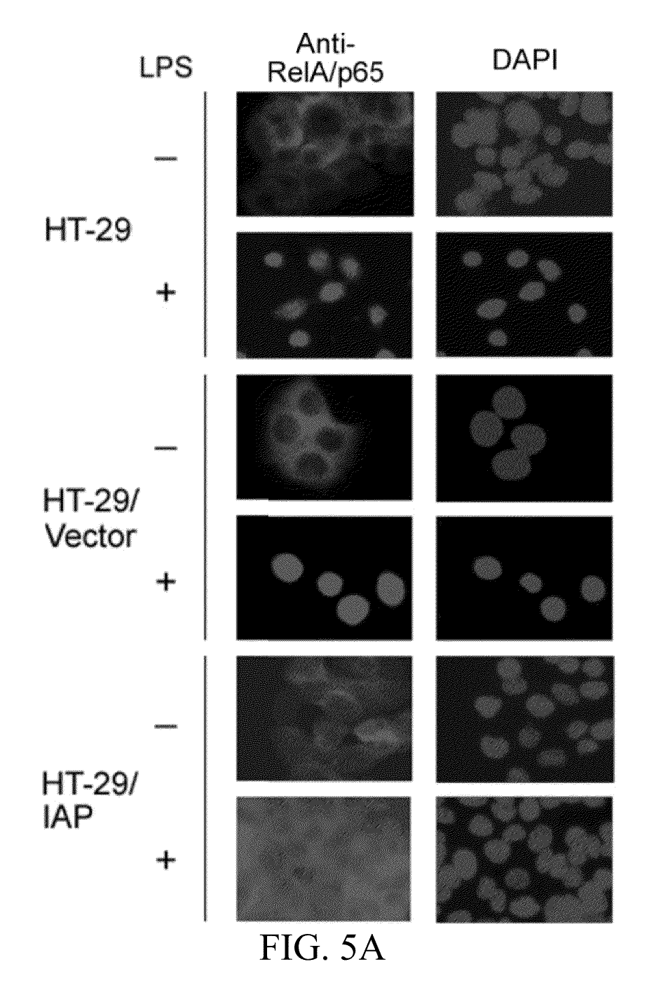

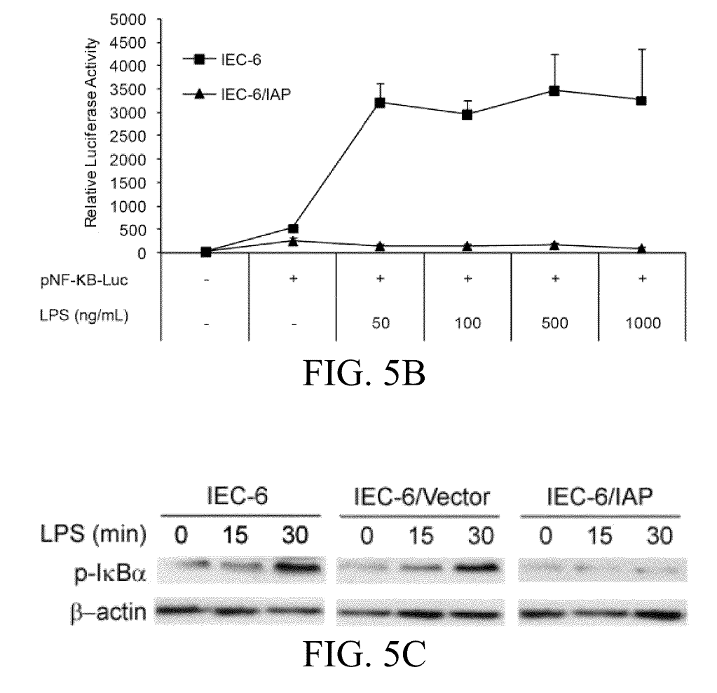

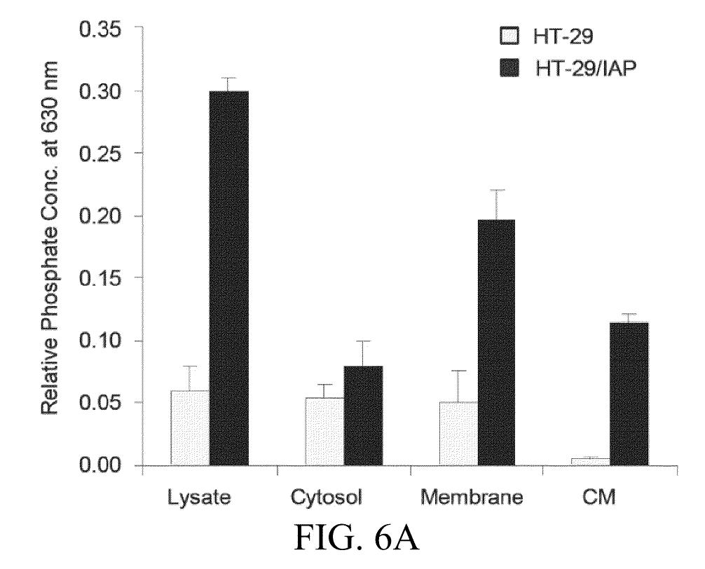

To assess the role of IAP in the intestinal barrier system against bacteria, stably-transfected intestinal cell lines expressing recombinant human IAP were produced. When parental cells (colorectal cancer cell line, HT-29) expressing no IAP were exposed to LPS, the LPS signaling was activated and the Rel/p65 complex was translocated to the nucleus, while the signaling was blocked in the transformant cells overexpressing IAP (FIG. 5A). A rat intestinal cell line IEC-6 and IEC-6 cells over expressing IAP were transfected with a firefly luciferase reporter gene under control of a NF-κB response element together with a normalizing plasmid expressing Renilla luciferase (Dual-Luciferase Reporter System, Promega). Exposure of cells to various LPS concentrations activated the firefly luciferase from NF-κB response element only in the parental cells: no activation was detected in IAP over-expressing cells (FIG. 5B). Cells were exposed to LPS (1 μg/mL) or vehicle for a period between 0 and 30 minutes to analyze the status of LPS signaling. Western blot analysis was performed on whole-cell lysates prepared using NE-PER Nuclear and Cytoplasmic Extraction Reagents kit from Pierce (Rockford, Ill.) and probed with an antibody specific for the cytosolic signaling protein, phosphorylated IκBα. The IEC-6 cells expressing IAP did not show increased IκBα phosphorylation indicating that LPS signaling was blocked (FIG. 5C).

ii. LPS Dephosphorylating Activity of Extracts from IAP Overexpressing Cells

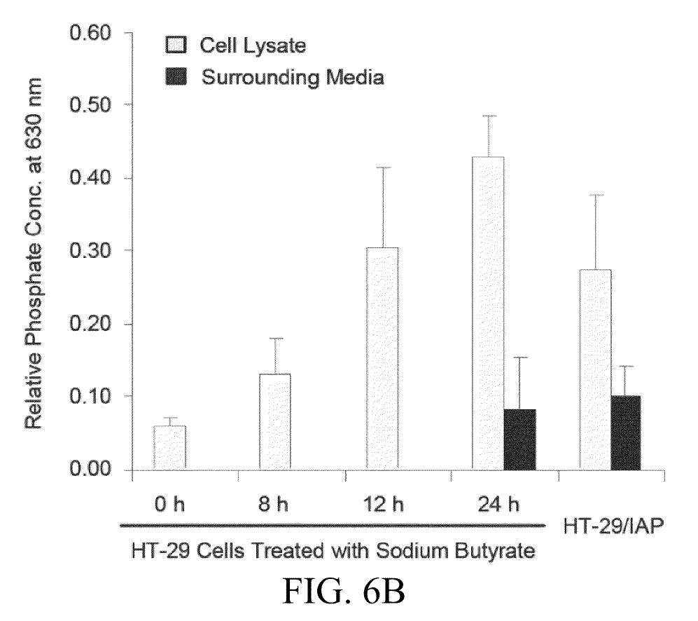

Parental HT-29 cells, HT-29/IAP transfectants and HT-29 cells treated with 5 mM sodium butyrate, which induces endogenous IAP by altering the methylation of nuclear DNA, were used to measure LPS dephosphorylating activity. Cells were first separated into cytosolic and membranous using the Mem-PER Eukaryotic Membrane Protein Extraction Kit (Pierce). MAPK activity was used as a cytosolic control. LPS (5 mg/mL) was added to the lysate for 2 hours, and then Malachite green solution (0.01% Malachite Green, 16% sulfuric acid, 1.5% ammonium molybdate and 0.18% Tween-20) was added and incubated for 10 minutes (Baykov, et al., 1988). Activity was then determined via spectrophotometric quantification taking background readings into account, and results were expressed in absorbance at 630 nm. The unfractionated whole lysate of HT-29/IAP cells showed high activity, and the membrane fraction contained most of the activity since the recombinant IAP contains a GPI anchor (FIG. 6A). Endogenous human IAP was induced in HT-29 parental cells by sodium butyrate treatment and samples from 24 hrs induction showed the same level of LPS dephosphorylating activity as the transformant cells (FIG. 6B). This result indicates that endogenous IAP as well as recombinant IAP expressed on the cell membrane can dephosphorylate LPS as a substrate.

iii. LPS Dephosphorylating Activity of Duodenum Samples from WT and Akp3^{-/-} Mice

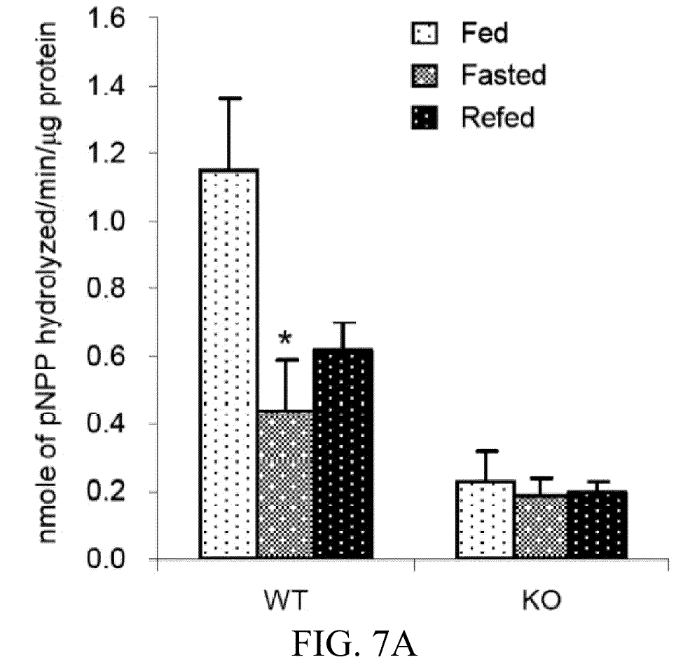

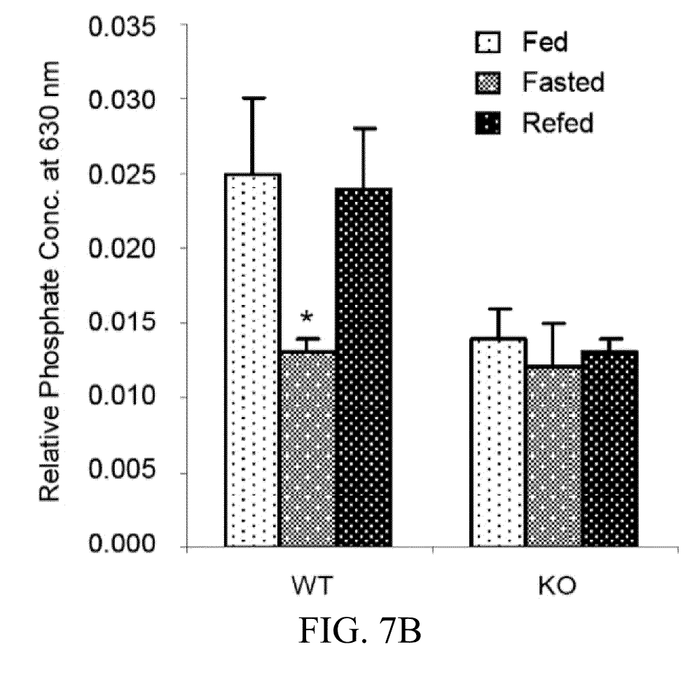

Duodenal mucosa from WT and Akp3^{-/-} littermate mice was extracted and LPS dephosphorylating activity was tested using the same procedure described above. Mouse duodenum strongly expresses dIAP, besides lower levels of gIAP and EAP. AP activity in the duodenum extracts using pNPP as a substrate is shown in the FIG. 7A. Remaining activity in the Akp3^{-/-} duodenum is mostly due to the expression of gIAP and very small amount of EAP. Fasting reduced the dIAP expression; however, re-feeding caused a rebound of the dIAP expression in WT mice. LPS dephosphorylating activities of the same samples are shown in FIG. 7B. The WT duodenum showed significantly higher LPS dephosphorylating activities compared to the knockout mice, and the activity returned after re-feeding. Thus, dIAP silencing could result in an impaired ability of the host to protect itself from luminal LPS exposure. The difference between WT and Akp3^{-/-} in the pNPPase activity (FIG. 7 A) is greater than that of LPS dephosphorylating activity (FIG. 7 B). This data indicates that both dIAP and gIAP can dephosphorylate LPS in vitro. The recombinant dIAP has much higher activity in a pNPP assay than does gIAP (Kₘ; dIAP vs gIAP: 1.1±0.34 vs 0.79±0.17). This can explain the differences seen in the pNPPase assay.

### 3. Example 3

### Screening Comprehensive Chemical Libraries to Identify Small Molecules that Specifically Modulate IAP's Enzymatic Activity

i. Methods

a. Production of Enzymes.

An expression vector pCMV-Script containing cDNA for human IAP, TNAP, PLAP, GCAP, mouse TNAP, dIAP, EAP or gIAP in secreted form (FLAG-tagged) is transfected into COS-1 cells for transient expression using a standard electroporation method. The GPI anchoring site is replaced by a FLAG sequence to make the proteins secreted into the media as well as to test their kinetics in a form immobilized by anti-FLAG antibody (Narisawa, et al, 2007). Medium is changed to serum free medium Opti-MEM (Invitrogen) 24 h later, and media containing secreted proteins was collected 66 hr after electroporation. Conditioned medium filtered by a 2 μm cellulose acetate membrane is supplemented with 0.1% BSA, aliquotted and stored at −80° C.

Human IAP is produced on a large scale to be used in the primary high throughput screening. For a maximum of 200,000 wells including a blank and negative control in each plate, approximately 1600 ml of the recombinant human IAP working solution (8 μl/well×200,000) is used. The working solution is a 1:80 dilution of the stock solution that has AP activity showing ΔOD405 (velocity) ˜300 in 5 min pNPP calorimetric assay. Therefore a minimum of 20 ml of the stock solution (1600÷80) is needed. An enzyme stock is prepared from ten 15 cm φ plates of COS-1 cells using 100 μg plasmid DNA [ten plates×(10 μg DNA/1×10⁷ cells per 15 cm φ plate)].

b. Assay Protocol.

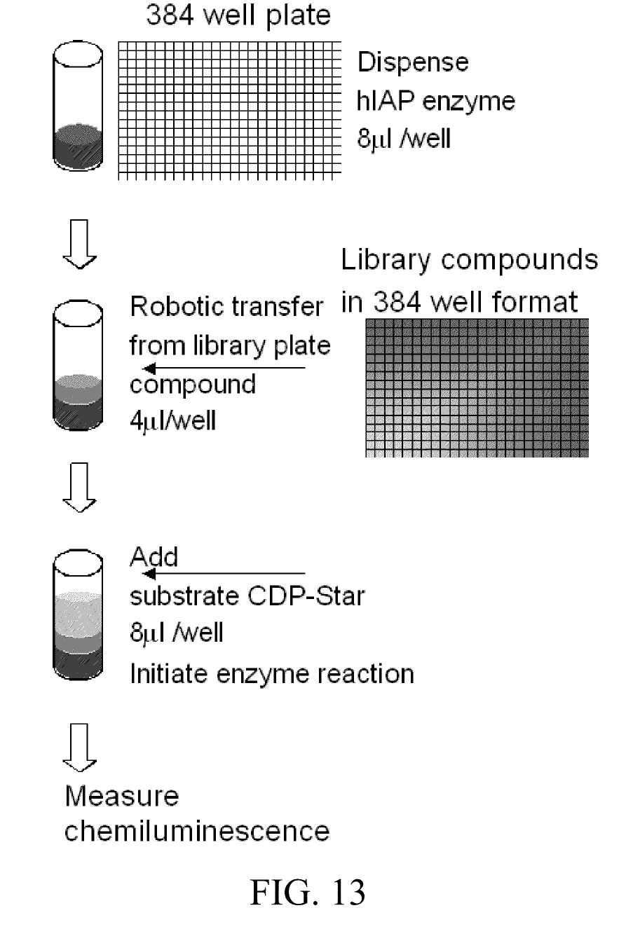

Compound aliquots (4 μL at 100 mM in 10% DMSO) are added to 8 μL of human IAP working solution of a the human IAP stock solution diluted 1:80 in assay buffer (250 mM DEA, pH 9.8, −2.5 mM MgCl₂, −0.05 μM ZnCl₂). The solution of substrate CDP-Star, disodium 2-chloro-5-(4-methoxyspiro{1,2-dioxetane-3,2′(5′-chloro)-tricyclo[3.3.1.1]3,7 decan}-4-yl)-1-phenyl phosphate (New England Biolabs), (8 μL of 125 μM in water) is added to each well. The CDP-Star system is chosen for the primary screening rather than the classic calorimetric assay using pNPP as a substrate, since the chemiluminescent reaction with CDP-Star has higher sensitivity and is not affected by the endogenous absorbance of some compounds in the library and/or of tissue extracts. The final concentration of CDP-Star (442.5 μM) is equal to its Kₘ value determined in assay buffer. Dispensing of human IAP working solution and CDP-Star is processed using a WellMate bulk dispenser (Matrix). Plates (white 384-well small volume Greiner 784075) are incubated at room temperature for 30 min, and the luminescence signal is measured using a PerkinElmer EnVision multi-mode plate reader. L-Phenylalanine (1 mM final concentration) and 2% DMSO is utilized as an inhibition control and blank, respectively. Data analysis is processed using CBIS software (ChemInnovations, Inc). The procedure is summarized in FIG. 13.

c. Strategy to Identify Activators.

The data analysis software used for the chemical library screening is designed to identify “inhibitors”; therefore, a positive number from the analysis means “positive inhibition”, while a negative number indicates “increased/activated enzymatic reaction.” Each of the compounds that gave negative values is manually tested in the primary screening in order to eliminate possible false signals/artifacts.

A dose dependency assay using the CDP-Star system is used for compounds that give a reproducible result in the manual test. The compound is diluted (100 mM to 0.03 mM) and incubated with the human IAP enzyme for 30 min prior to addition of substrate. At the same time, human TNAP, human PLAP, human GCAP, mouse TNAP, mouse dIAP, mouse EAP and mouse gIAP is tested to determine enzyme specificity. The amount of each enzyme is standardized to the AP activity that gives ˜0.5 ΔOD 405 (velocity) for a 30 min reaction, and the final data plotted as % change from the original value with 0 mM compound.

d. Interpretation

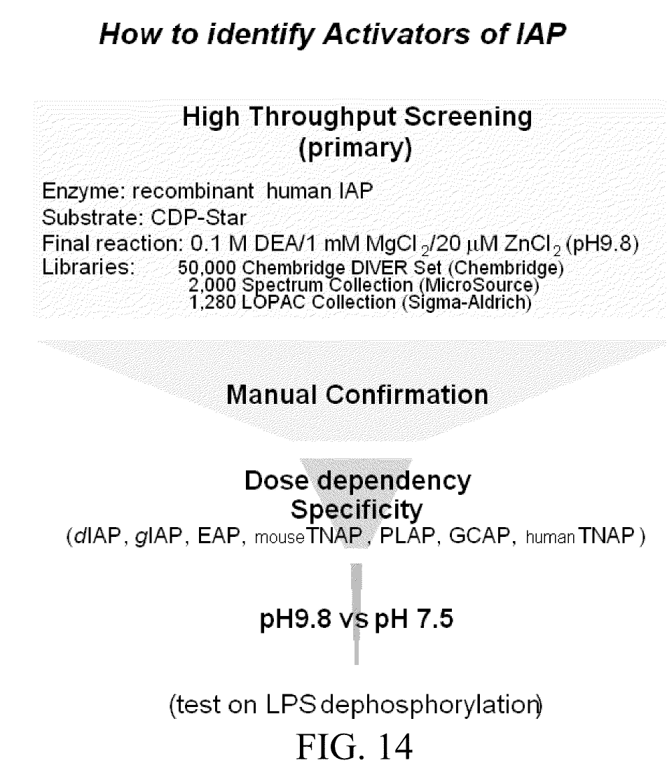

Enzyme inhibitors are often categorized as allosteric, competitive, uncompetitive or noncompetitive; however, interaction of enzyme activators towards the enzyme and substrate can differ from inhibitors. It is desired to identify a molecule that works in vivo. The kinetics are therefore compare at pH 9.8 and pH 7.5. An assay at neutral pH represents the in vivo situation more effectively (Narisawa, et al., 2007). Alkaline pH is used for the primary screening because the sensitivity at neutral pH is too low to be used for in the robotic system. Therefore, the behavior of the obtained activators can be tested at neutral pH at this step. A summary of the screening strategy is shown in FIG. 14.

### 4. Example 4

### Effect on LPS Dephosphorylating Activity In Vitro

i. Methods

a. Effect on LPS Dephosphoylating Activity In Vitro Using Recombinant IAPs

Solutions of recombinant enzymes, FLAG-tagged human IAP, mouse dIAP, mouse EAP and mouse gIAP are standardized to the AP activity that gives ˜0.5 ΔOD 405 (velocity) for a 5 min pNPP calorimetric assay, and are incubated in a 96-well plate coated with anti-FLAG antibody (Sigma). The plate is washed with TBS-0.1% Tween 20. Activators at concentration 0, 3.3, 10, 30 μM together with 5.0 mg/ml LPS from Escherichia coli (0111: B4, Fluka) which is prepared in 20 mM TrisHCl (pH7.5)-150 mM NaCl-1 mM MgCl₂-20 μM ZnCl₂, is incubated in the wells for 2 hours. Biomol Green reagent (Malachite green/ammonium molybdate solution, Baykov, et al 1988) is added to measure released Pi. All points will be done in triplicate. Wells without enzyme are used as a background to be subtracted.

b. Effect on LPS Dephosphorylating Activity In Vitro Using Intestinal Samples

WT and Akp3^{-/-} mice aged 8-16 weeks (each pair is from gender matched littermates) are euthanized by CO₂ gas. Small intestines are dissected immediately and opened up longitudinally in ice cold TBS (20 mM TrisHCl (pH7.5)-150 mM NaCl) to remove the ingesta. The intestines are divided into four segments (25% length each from proximal to distal; Segments 1, 2, 3, 4). Segment 1 represents duodenum, Segments 2 and 3 are mainly jejunum and Segment 4 is mostly ileum. Each segment is placed in a tube containing 2 ml extraction buffer [50 mM TrisHCl (pH 8.9)-1 mM MgCl₂-20 μM ZnCl₂-0.1% TritonX-100] and 2 ml of n-buthanol. After brief vortexing and 15 min rotation, tubes are spun at 1,000 g for 10 min. The aqueous phase containing alkaline phosphatases released from intestinal villi will be further centrifuged at 100 Kg for 15 min to remove debris. Protein concentration will be determined by BCA (Pierce), and all samples will be adjusted to 1.5 mg/ml with extraction buffer. Samples will be incubated in wells of a 96-well plate coated with a rabbit antibody (#3776), which was raised against recombinant gIAP but cross reacts with dIAP (Narisawa, et al., 2007). The plate is washed with TBS-0.1% Tween 20, and 5.0 mg/ml LPS from Escherichia coli (0111: B4, Fluka) is incubated in the wells for 2 hours. Biomol Green reagent (Malachite green/ammonium molybdate solution, Baykov, et al 1988) is added to measure released Pi. All points are done in triplicate. The negative control wells (no intestinal buthanol extract, no activator) are used as a background to be subtracted. The intestinal samples that give LPS dephosphorylating activity in the preliminary assay are incubated with each activator at 0, 3.3, 10, 30 μM together with 5.0 mg/ml LPS for 2 hours prior to the Biomol assay.

c. Interpretation

The activator's effect on LPS assay using recombinant IAPs can be equivalent to the results obtained from the CDP-Star assay at neutral pH above, since LPS is prepared in a buffer with neutral pH. Assay without activators in the LPS assay using intestinal extracts can determine whether dIAP and gIAP have same ability to dephosphorylate LPS. Segment 1 (duodenum) extract from WT mice contains both dIAP and gIAP, and Segment 2, 3, 4 extracts contain gIAP, while all the samples from the Akp3^{-/-} mouse contain gIAP. If only Segment 1 from WT mice shows LPS dephosphorylating activity, then dIAP is the major detoxifier of LPS. If other segments from both WT and Akp3^{-/-} show significant values, and an independent assay using a rabbit antibody to dIAP (#8933) with no crossreactivity to other mouse APs, shows a negative value, gIAP can have a role in detoxifying LPS. The jejunum and ileum samples with antibody #8933 can serve as a negative control. If the negative controls, Akp3^{-/-} mice, still show significant activity, EAP can be examined. Samples are incubated with anti-EAP antibody (#8936) and Akp5^{-/-} mice used (Narisawa, et al., 1997) as negative control.

### 5. Example 5

### Effect of Activators on Intestines of Wild and Akp3^{-/-} Mice Exposed to LPS

i. Methods

a. Absorption, Distribution, Metabolism and Elimination (ADME) Parameters:

The assessment of a molecule's ADME profile provides the optimal means of discovering potential issues with respect to bioavailability and in vivo efficacy. The following assays and screens are utilized to prioritize new drug candidates having optimal predicted in vivo characteristics.

Microsomal Stability: The microsomal stability assay uses specific liver microsomes to give essential information on a compound's potential to be metabolized by the liver. To do this, the compound solution is incubated with species-specific liver microsomes for up to 45 minutes at 37° C. The reactions are terminated at 5 time-points with the addition of methanol containing an internal standard. Following protein precipitation and centrifugation, the samples are analyzed by LC-MS/MS.

Cytochrome P450 Inhibition: The cytochrome P450 inhibition assay quantifies the extent that a pharmaceutical compound inhibits the key cytochrome P450 enzymes. Inhibition of these enzymes can predict potential drug-drug interactions. For this procedure, the compound is incubated with microsomes and NADPH in the presence of a specific cytochrome P450 probe substrate. After the incubation period, methanol containing internal standard is added to stop the reaction. For the various isoforms (CYP2C9, CYP2C19, CYP2D6 and CYP3A4), the metabolites are monitored using LC-MS/MS. A decrease in the formation of the metabolite compared to the vehicle control is used to calculate the IC₅₀ value. Known selective P450 inhibitors are included as control reactions alongside the test compounds to assess the validity of the result.

Permeability: The Parallel Artificial Membrane Permeation Assay (PAMPA) measures the passive diffusion of a test compound through an artificial hexadecane membrane. The protocol was designed to predict passive, transcellular permeation of a drug substance. The compound solutions (in buffer, minimal DMSO) are filtered before addition to the donor compartment of the plate. Permeation through the pre-prepared artificial membrane into the receiver compartment is measured following a 5-hour incubation at room temperature. Analytical standards are prepared from the filtered test compound solutions. Compounds are quantified by LC-MS/MS analysis, using a 5-point calibration, with appropriate dilution of the samples. Up to four apparent permeability coefficients for each compound are calculated along with the experimental recovery.

b. Short-Term In Vivo Test.

The activator is prepared in 0.2 ml PBS and 0, 3 and 9 mg/kg body weight and will be given by gavage. L-phenylyalanine, a known IAP inhibitor, is administered (40 mg/kg) to a negative control group. Ten minutes later, LPS (0111: B4, Fluka) dissolved in 0.2 ml PBS is administered to activator-treated mice at 20 mg/kg body weight by gavage. Mice are anesthetized with Avertin (IP, 15 μl/g) 2 hours later to collect blood by cardiac puncture and intestinal tissues. Intestinal segments are analyzed by Western blots to test activation of LPS signaling using antibodies against phosphorylated IκBα and phosphorylated NF-κB/p65. A part of intestinal segments are fixed in 4% paraformaldehyde and used for immunohistochemistry to compare nuclear translocation of p65 (DePlaen, et al., 2000). Levels of active LPS in serum are measured by a Limulus amebocyte lysate based LPS detection kit, Pyrochrome Chromogenic Test kit (Cape Cod, Inc.). All gavage experiments are done in triple pairs of WT and Akp3^{-/-} mice aged 8-16 weeks (each pair is from gender matched littermates).

c. 24 Hour In Vivo Test

Mice are housed with water bottles containing activator compound (0, 100 or 300 μg/ml). Total intake of activator in 24 hours should be ˜0, 0.8, or 2.4 mg, since one C57B1/6 mouse with 30 g body weight drinks approximately 8 ml water in 24 hour (Bachmanov, et al, 2002). LPS prepared in PBS is administrated by gavage (20 mg/kg), and the water bottle containing activator renewed at the same time. Twenty-four hours later, mice drinking activator areanesthetized with Avertin (IP, 15 μl/g) and blood and intestinal tissue collected. Samples are processed for LPS measurement, Western blots and immunohistochemistry as well as the short-term in vivo test. All gavage experiments are done in triple pairs of WT and Akp3^{-/-} mice aged 8-16 weeks (each pair is from gender matched littermates).

TABLE 3

| Test | Activator | LPS | Collection |
|---|---|---|---|
| Short term in | t = −10 min | t = 0 | t = 2.0 hr |
| vivo test | 0, 3, 9 mg/kg gavage | 20 mg/kg | |
| | | gavage | |
| 24 hr in | t = −24 hr till 24 hr | t = 0 | t = 24 hr |
| vivo test | 0, 100, 300 μg/ml water | 20 mg/kg | |
| | bottle | gavage | |

d. Interpretation

The short-term test shows IAP activation effect when high concentration of activator is present at the time of LPS exposure such as in duodenum. If lowered levels of active LPS in the serum and LPS signaling are seen in WT mice with an activator than WT without an activator, while they are increased in Akp3^{-/-} mice with and without activator, this activator is helping dIAP to detoxify LPS. The 24 hr test is to look at an effect of IAP activators on LPS exposure occurring extended period in the entire intestines. An activator that shows positive results in the 24 hr test as well as the short-term test is a desirable molecule because the 24 hr test indicates that it maintains the efficacy for long period with relatively low concentration.

An activator that prevents/reduces LPS signaling in WT animals but has no effect in Akp3^{-/-} animals can be interpreted to activate dIAP expressed in the duodenum. If both WT and Akp3^{-/-} animals show reduced LPS signaling with an activator, while WT and Akp3^{-/-} mice with L-phenylalanine show increased LPS signaling, this compound can be activating gIAP/EAP expressed in the entire small intestine and promoting LPS dephosphorylation. In this case, ileum samples from Akp5^{-/-} animals that contain only gIAP can be examined.

### 6. Example 6

### Protecting the Gastrointestinal Tract Against Bacterial Insult and Tumorigenesis

A critical function of the mammalian intestinal mucosa is to provide a barrier to luminal microbes and toxins, while allowing digestion and absorption of nutrients. It is evident that under conditions of starvation and/or disease, the intestinal barrier becomes impaired, leading to significant morbidity and mortality (Muller et al., 2005 Cell Mol Life Sci 62: 1297-130). Intestinal Alkaline Phosphatase (IAP), a brush-border enzyme is expressed exclusively in villus-associated enterocytes. IAP expression is down-regulated by fasting, while tropic enteral feeding restores IAP expression (Hodin et al., 1994, Am J Physiol 266: G83-G89). Several studies have shown that IAP can detoxify bacterial lipopolysaccharide (LPS)—a major cell wall component of gram-negative bacteria—through dephosphorylation of the lipid A structure, which is the primary source of its endotoxic effect (Poelstra et al., 1997 Carcinogenesis: 1567-1572; Bentala et al., 2002, Shock 18: 561-566). LPS exposure induces IAP expression (Kapojos et al., 2003, Int. J. Exp. Pathol. 84: 135-144). Previous studies have shown that IAP expression is initiated when a drastic population change of intestinal flora from neonatal to adult type occurs prior to weaning and that IAP acts as a mucosal defense factor against bacterial invasion (Narisawa et al., 2007, Mol. Cell. Biol. 23: 7525-7530; Bates et al., 2007, Cell Host and Microbe 2: 371-382; Goldberg et al., 2008, Proc. Natl. Acad. Sci. USA 105: 3551-3556). In IAP^{-/-} mice the bacterial invasion is severe after ischemia/reperfusion—a clinically relevant model of intestinal injury with inflammation—supporting the concept that the beneficial effect of tropic enteral feeding observed in critical illness is a result of maintenance of IAP function (Goldberg et al., 2008, Proc. Natl. Acad. Sci. USA 105: 3551-3556). Furthermore, the association between high levels of LPS in the gut and the development of Inflammatory Bowel Disease (IBD) is well-established (Loftus et al., 2002, Alimentary Pharmacology & Therapeutics 16: 51-60) and IAP administration has been proposed as a treatment for IBD (Poelstra et al., 1997,Am. J. Pathol. 151: 1163-1169; Beumer et al., 2002, J. Pharmacol. Exp. Ther. 307: 737-744).

Colorectal cancer, a frequent malignant tumor is a major cause of death in the Western hemisphere, and develops spontaneously or as a long-term complication of chronic bowel inflammation such as in Crohn's Disease, ulcerative colitis and IBD (Xie and Itzkowitz, 2008, World J Gastroenterol. 14: 378-389). Colorectal cancer can be studied using a mouse model of colitis-associated cancer, i.e., azoxymethane (AOM)-induced colonotropic carcinogenesis, which closely resembles colorectal cancer in man. Azoxymethane (AOM) is a chemical agent that can initiate cancer by alkylation of DNA, thereby facilitating base mispairings (Papanikolau et al., 1998, Carcinogenesis 21: 1567-1572). AOM itself does not represent the final carcinogenic metabolite, it is stepwise activated including a hydroxylation step mediated by cytochrome P450 in the liver (Sohn et al., 2001, Cancer Res. 61: 8435-8440) and, after secretion in the bile, it is further metabolized by the colonic flora (Fiala et al., 1977, Cancer 40, 2436-2445; Reddy et al., 1974, Cancer Res. 34: 2368-2372). While repeated administration of AOM alone can drive spontaneous tumor formation, tumor formation is greatly accelerated by the pro-inflammatory agent dextran sodium sulfate (DSS) (Tanaka et al., 2003, Cancer Sci. 94: 965-973; Neufert et al., 2007, Nature Protocols 8: 1998-2001). Combined, a single AOM injection and DSS generates a model of colitis-associated tumor development. Twin studies in humans have shown a strong genetic component for the sensitivity to gastrointestinal inflammatory disease and tumor development is in turn associated to inflammation resulting from loss of integrity of the gastrointestinal epithelium and the particular bacterial population profile permitted by the host genetic background (De la Chapelle, 2004). IAP can alter the risk of colon cancer development by altering the metabolism of toxins or by altering sensitivity to inflammation caused by compromised epithelial integrity.

As shown herein the level of IAP has been linked to bacterial insult and the onset of colorectal cancer. Results show that a decreased level of IAP results in an increase level of bacterial insult in the gastrointestinal tract and also an increased risk of obtaining colorectal cancer.

i. Methods

An IAP knockout mouse model was previously developed and characterized (Narisawa et al., 2003, Mol. Cell. Biol. 23: 7525-7530). Furthermore, it was previously determined that the IAP expression in the murine gut starts just prior to weaning, a time that coincides with a change in the gastrointestinal flora (Narisawa et al., 2007,Am. J. Physiol. Gastrointest. Liver Physiol. 293: 1068-1077). The sensitivity of IAP^{-/-} mice was also studied during ischemic insults known to cause a breakdown in mucosal defense against endogenous luminal bacteria/toxins (Goldberg et al., 2008, Proc. Natl. Acad. Sci. USA 105: 3551-3556).

ii. Results

a. Bacterial Counts in WT and KO Mice

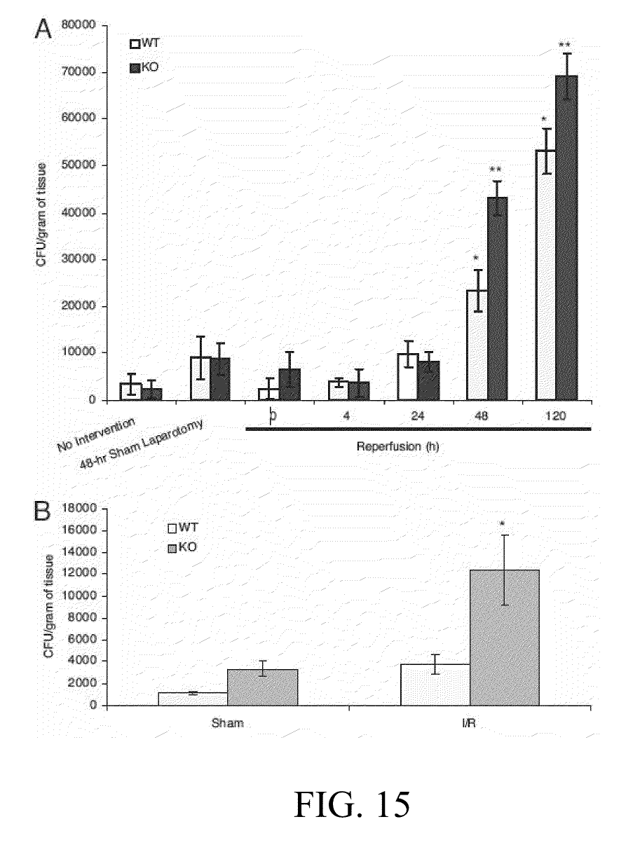

Both WT and KO animals were studied using the ischemia/reperfusion (I/R) model as it is a standard technique of superior mesenteric artery ligation followed by reperfusion (Hinnebusch et al., 2002, J Gastrointest Surg 6: 403-409). WT and IAP KO mice were exposed to 45 min of superior mesenteric ligation clamping followed by varying times of reperfusion. Sham laparotomy and no intervention were used as controls. Mesenteric tissues were harvested, and bacterial counts in the nodes were determined. Sham mice were used for control purposes in all experiments. It is clear from the data that IAP protects the mice from gut bacterial translocation. Although the gut barrier became disrupted in both the WT and KO animals, the presence of IAP prevented much of the bacteria from crossing the mucosal barrier and entering the mesenteric lymph nodes (see, FIG. 15).

b. 9 Week AOM/DSS Tumor Model Used in WT and Ets2^{A72/A72} Mice

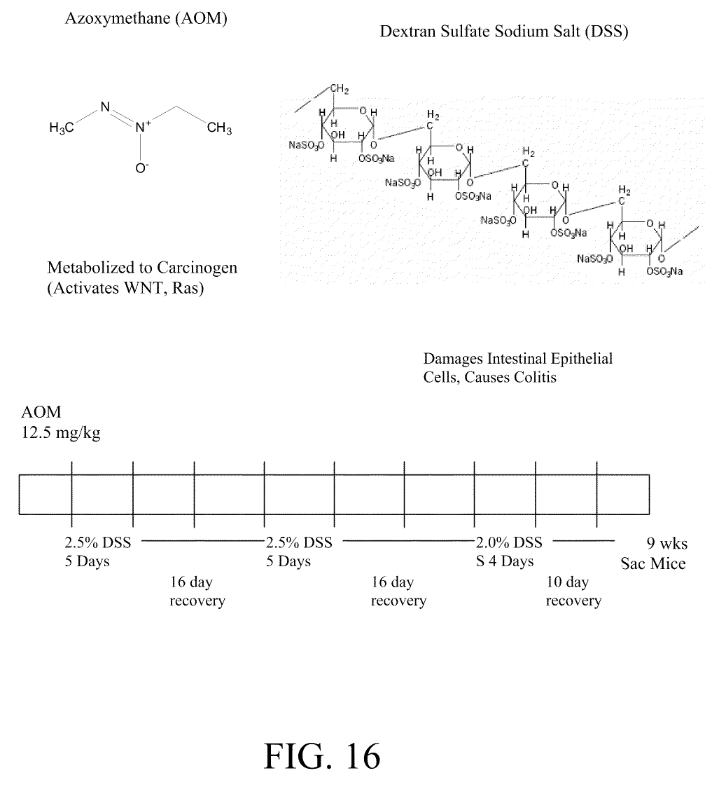

An AOM/DSS tumor model, was used to determine the effect of IAP and tumor formation in mice. AOM was administered to both WT and Ets2^{A72/A72} mice which was followed by 5 days of DSS administration followed by recovery periods. 6-8-week old IAP Ets2^{A72/A72} and WT sibling control mice intraperitoneally (i.p.) with 12.5 mg/kg of AOM or PBS (vehicle alone). After 5 days, the mice was put on a cycle of 2.5% dextran sodium sulfate (DSS) in their drinking water for 5 days followed by 16 days of regular water. The cycle wwa be repeated once more. In the final cycle the mice was given 2% DSS for 4 days followed by 10 days of regular water (see, FIG. 16). During treatment the mice are weighed daily and visually inspected for diarrhea and rectal bleeding. At the end of the experimental period, all mice are sacrificed, and the colon, spleen and mesenteric lymph nodes was be collected for histological examination. Diarrhea and occasional rectal bleeding are consequences of colitis and these parameters were monitored to detect the onset and progress of disease. The mice typically continue to lose weight 3-4 days after DSS but will recover subsequently. All animals that appear dehydrated was treated with subcutaneous lactated Ringer's solution.

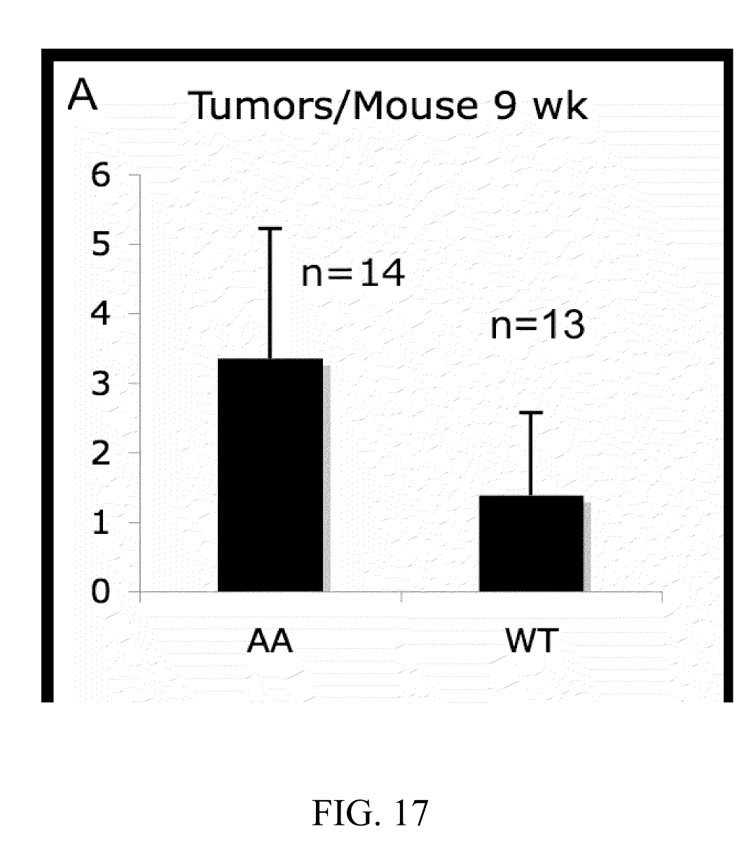

In 69% (9/13) of the WT mice macroscopic adenomas developed. In Ets2^{A72/A72} mice 92% (13/14) of the animals developed tumors by 9 weeks. This is a 33% increase in tumor formation in Ets2^{A72/A72} animals compared to WT. Furthermore, Ets2^{A72/A72} animals developed 3 times as many tumors per animal compared to WT animals (see FIG. 17) Histological analysis confirmed that the tumors were adenomas.

c. 19 Week AOM/DSS Tumor Model Used in WT and Ets2^{A72/A72} Mice

The same AOM/DSS tumor model as described above, was used to determine the effect of IAP and tumor formation in mice. AOM was administered to both WT and Ets2^{A72/A72} mice which was followed by 5 days of DSS administration followed by recovery periods. This cycle was repeated three times (9 weeks) and the animals were permitted to develop tumors for an extra 10 weeks before the animals were sacrificed and organs were harvested.

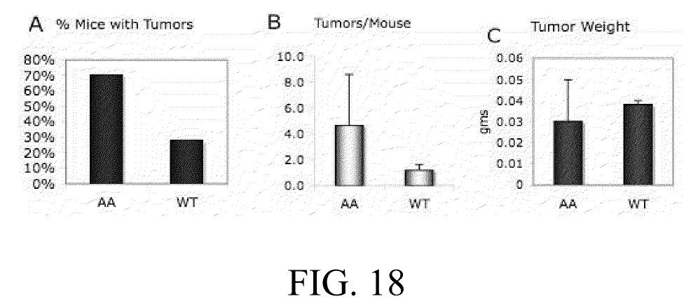

70% of the Ets2^{A72/72} (AA) animals developed tumors while less than 30% of the control animals developed tumors (FIG. 18A). Again, the average number of tumors per animal was much greater in the Ets2^{A72/A72} mice. Each Ets2^{A72/A72} mice had about 5 tumors while the WT mice only had about 1 tumor (see FIG. 18B). However the average tumor size for the two types of mice were not significantly different (FIG. 18C).

Even though there is a decrease in the number of tumors in WT mice compared to Ets2^{A72/A72} mice the size of each tumor appears to be similar. This indicates that the sensitivity of Ets2 deficient mice to colon tumor formation may be due to early transforming events rather than tumor growth.

iii. Discussion

The AOM/DSS tumor model can also be used for WT and IAP^{-/-} mice to determine if the IAP is a significant mediator or tumor formation. The study can be performed as described above without modifications. Inflammatory Bowel Disease (IBD) is linked to IAP and IBD is linked to colorectal cancer which indicates that the level of IAP can directly be related to the onset of colorectal cancer. Therefore, the present discovery of compounds that increases the IAP level allows reduction of the risk of IBD and colorectal cancer.

A robust LPS-dephosphorylation assay suitable for HTS in search of small molecule compounds able to “activate”/enhance IAP activity can be developed. The assay can use human IAP for the screen to secure “activators” that can be useful for future development as therapeutic drugs. In a secondary screen, the primary hits would be tested for their ability to also activate mouse IAP, which will enable follow up studies in the AOM/DSS mouse models. An ex vivo confirmatory screen can also be used in a third instance, since the glycosylation pattern of the human and mouse recombinant enzymes are sure to differ from the patterns found in the enterocytes, and that variability is known to affect the catalytic activity of IAP (Narisawa et al., 2007, Liver Physiol. 293: 1068-1077).

The identified compounds that activate both human and mouse IAPs can be evaluated in experimental mouse models while being further optimized for clinical trials with minimal delay. Compounds that show significant activation of LPS dephosphorylation in the in vitro assay will be chosen for ex vivo studies using gastrointestinal segments of WT and IAP^{-/-} mice, since it was previously established that the LPS dephosphorylating activity in the gastrointestinal tract from WT mice and found that the activity was greatly reduced in the IAP^{-/-} duodenum (Goldberg, et al., 2008, Proc. Natl. Acad. Sci. USA 105: 3551-3556). Small molecule activators will enhance LPS detoxification in specimens from WT animals that express IAP activity while no effect would be observable in mice lacking IAP function.

### F. REFERENCES

Alpers D H. Enteral feeding and gut atrophy. Current Opinion in Clinical Nutrition & Metabolic Care 5: 679-683, 2002.
Bachmanov A A, Reed D R, Beauchamp G K, Tordoff M G. Food Intake, Water Intake, and Drinking Spout Side—Preference of 28 Mouse Strains. Behavior Genetics 32: 435-443, 2002.
Bates J M, Akerlund J, Mittge E, Guillemin K. Intestinal Alkaline Phosphatase Detoxifies Lipopolysaccharide and Prevents Inflammation in Zebrafish in Response to the Gut Microbiota. Cell Host & Microbe 2: 371-382, 2007.
Baykov A A, Evtushenko O A, Avaeva S M. A malachite green procedure for orthophosphate determination and its use in alkaline phosphatase-based enzyme immunoassay. Analytical Biochemistry, 171: 266-270, 1988.
De Plaen I G, Tan X-D, Chang H, Wang L, Remick D G, Hsuch W. Lipopolysaccharide activates nuclear factor κB in rat intestine: role of endogenous platelet-activating factor and tumour necrosis factor. British Journal of Pharmacology 129: 307-314, 2000.
Eberl G. Inducible lymphoid tissues in the adult gut: recapitulation of a fetal developmental pathway? Nature Reviews Immunology 5, 413-420, 2005.
Fukuda M N. CRC Handbook of Endoglycosidases and Glycoaminases, edited by Takahashi N and Muramatsu T. Boca Raton, Fla.: CRC, 1992, p. 55-103.
Fukushima K, Sasaki I, Takahashi K, Naito H, Ogawa H, Sato S, Matsuno S. Lipopolysaccharide Exhibits Synergistic Enhancement of Butyrate-Induced and Retinoic Acid-Mediated AlkalinePhosphatase Activity on Small Intestinal Epithelial Cell Line, IEC-6. Digestion 59: 683-688,1998.
Gardiner K R, Halliday M I, Barclay G R, Milne L, Brown D, Stephens S, Maxwell R J, Rowlands B J. Significance of systemic endotoxaemia in inflammatory bowel disease. Gut 36: 897-901, 1995.
Glickman R M, Alpers D H, Drummey G D. Isselbacher KJIncreased lymph alkaline phosphatase after fat feeding: effects of medium chaintriglycerides and inhibition of protein synthesis. Biochim Biophys Acta 201: 226-235, 1970.
Goldberg R F, Austen, Jr W G, Zhang. X, Munene, G, Mostafa G, Biswas S, McCormack M, Eberlin K R, Nguyen J T, Tatlidede H S, Warren H S, Narisawa S, Millán J L, Hodin R A. Intestinal Alkaline Phosphatase is a Gut Mucosal Defense Factor Maintained by Enteral Nutrition. Proceedings of the National Academy of Sciences of the United States of America, In Press 2008.
Gorbach S L. Intestinal microflora. Gastroenterology 60:1110-1129, 1971.
Hahnel A C, Rappolee D A, Millán J L, Manes T, Ziomek C A, Theodosiou N G, Werb Z, Pedersen R A, Schultz G A. Two alkaline phosphatase genes are expressed during early development in the mouse embryo. Development 110: 555-564, 1990.
Hessle L, Johnson K A, Anderson H C, Narisawa S, Sali A, Goding J W, Terkeltaub R, Millán J L. Tissue-nonspecific alkaline phosphatase and plasma cell membrane glycoprotein-1 are central antagonistic regulators of bone mineralization. Proceedings of the National Academy of Sciences of the United States of America 99: 9445-9449, 2002.
Hodin R A, Graham J R, Meng S and Upton M P. Temporal pattern of rat small intestinal gene expression with refeeding. Am J Physiol Gastrointest Liver Physiol 266: G83-G89, 1994.
Hooper L V and Gordon J I. Commensal Host-Bacterial Relationships in the Gut. Science 292 (11) 1115-1118, 2001.
Horrigan F D, Danovitch S H. The origin of human fecal alkaline phosphatase. American Journal of Digestive Diseases 19: 603-608, 1974.
Hoylaerts M F, Ding L, Narisawa S, Van kerckhoven S, Millán J L. Mammalian Alkaline Phosphatase Catalysis Requires Active Site Structure Stabilization via the N-Terminal Amino Acid Microenvironment. Biochemistry 45: 9756-9766, 2006.
Johnson K A, Hessle L, Vaingankar S, Wennberg, C, Mauro, S Narisawa, S Goding J W, Sano K, Millán J L, Terkeltaub R. Osteoblast tissue-nonspecific alkaline phosphatase antagonizes and regulates PC-1. American Journal of Physiology—Regulatory Integrative & Comparative Physiology. 279: R1365-R1377, 2000.
Kobayashi K S, Chamaillard M, Ogura Y, Henegariu O, Inohara N, Nunez G Flavell R A. Nod2-dependent regulation of innate and adaptive immunity in the intestinal tract. Science 307: 731-734, 2005.
Koyama I, Matsunagab T, Haradab T, Hokari S, Komoda T. Alkaline phosphatases reduce toxicity of lipopolysaccharides in vivo and in vitro through dephosphorylation. Clinical Biochemisty 35:455-461, 2002.
Lee A and Gemmell E. Changes in the Mouse Intestinal Microflora During Weaning: Role of Volatile Fatty Acids. Infection and Immunity 5: 1-7, 1972.
Lin H C. Small Intestinal Bacterial Overgrowth, A Framework for Understanding Irritable Bowel Syndrome. JAMA 292, 852-858, 2004.
Loftus E V, Schoenfeld P, Sandborn W J. The epidemiology and natural history of Crohn's disease in population-based patient cohorts from North America: a systematic review. Alimentary Pharmacology & Therapeutics 16 (1): 51-60, 2002.
Manes T, Hoylaerts M F, Muller R, Lottspeich F, Holke W, Millán J L. Genetic complexity, structure, and characterization of highly active bovine intestinal alkaline phosphatases. Journal of Biological Chemistry 273: 23353-23360, 1998.
Martinez-Medina M, Aldeguer X, Gonzalez-Huix F, Acero D, Garcia-Gil L J. Abnormal microbiota composition in the ileocolonic mucosa of Crohn's disease patients as revealed by polymerasechain reaction-denaturing gradient gel electrophoresis. Inflammatory Bowel Diseases 12: 1136-1145, 2006.
McComb R B, Bowers G N Jr, Posen S. Alkaline Phosphatase. New York: Plenum, 1979.
Nakano M, Sumi Y, Miyakawa M. Isolation and properties of fecal proteins and fecal alkaline phosphatase from germfree and conventional rats. Applied and Environmental Microbiolgy 35:283-289, 1978.
Nakano T, Inoue I, Koyama I, Kanazawa K, Nakamura K, Narisawa S, Tanaka K, Akita M, Masuyama T, Seo M, Hokari S, Katayama S, Alpers D H, Millán J L, Komoda T. Disruption of the murine intestinal alkaline phosphatase gene Akp3 impairs lipid transcytosis and induces visceral fat accumulation and hepatic steatosis. Am J Physiol Gastrointest Liver Physiol 292: G1439-G1449, 2007.
Narisawa S, Frohlander N, Millán, J L. Inactivation of two mouse alkaline phosphatase genes and establishment of a model of infantile hypophosphatasia. Developmental Dynamics 208: 432-446, 1997.
Narisawa S, Harmey D, Yadav M C, O'Neill W C, Hoylaerts, M F, Millán J L. Novel inhibitors of alkaline phosphatase suppress vascular smooth muscle cell calcification. Journal of Bone & Mineral Research 22: 1700-1710, 2007.
Narisawa S, Hofmann M C, Ziomek C A, Millán J L. Embryonic alkaline phosphatase is expressed at Mphase in the spermatogenic lineage of the mouse. Development 116: 159-165, 1992.
Narisawa S, Hoylaerts M F, Doctor K S, Fukuda M N, Alpers D H, Millán J L. A novel phosphatase upregulated in Akp3 knockout mice. Am J Physiol Gastrointest Liver Physiol 293: G1068-G1077, 2007.
Narisawa S, Huang L, Iwasaki A, Hasegawa H, Alpers D H, Millán J L. Accelerated Fat Absorption in Intestinal Alkaline Phosphatase Knockout Mice. Molecular and Cellular Biology, 7525-7530, 2003.
Ozaki C K, Chu ShW, Walker W A. Developmental changes in galactosyltransferase activity in the rat small intestine. Biochim Biophys Acta 991: 243-247, 1989.
Riordan S M, McIver C J, Wakefield D, Duncombe V M, Thomas M C, and Bolin T D. Small intestinal mucosal immunity and morphometry in luminal overgrowth of indigenous gut flora. The American Journal of Gastroenterology 96: 494-500, 2001.
Ruemmele F M, Beaulieu J F, Dionne S, Levy E, Seidman E G, Cerf-Bensussan N, Lentze M J. Lipopolysaccharide modulation of normal enterocyte turnover by toll-like receptors is mediated by endogenously produced tumor necrosis factor α.Gut 51: 842-848, 2002.
Sears C L. A dynamic partnership: Celebrating our gut flora. Anaerobe 11: 247-251, 2005.
Strober W, Nakamura K. Kitani A. The SAMP1/Yit mouse: another step closer to modeling human inflammatory bowel disease. Journal of Clinical Investigation 107: 667-670, 2001.
Xie Q and Alpers D H. The two isozymes of rat intestinal alkaline phosphatase are products of two distinct genes. Physiological Genomics 3: 1-8, 2000.

1. A method of preventing gastrointestinal bacterial invasion in a subject, comprising administering to the subject an effective amount of an intestinal alkaline phosphatase (IAP) modulator.

2. A method of treating or preventing a disease or condition caused or exacerbated by gram-negative bacteria acting on the gastrointestinal mucosa, comprising administering to the mucosa an effective amount of an intestinal alkaline phosphatase (IAP) modulator.

3. The method of claim 1, wherein the IAP modulator is an IAP activator.

4. The method of claim 1, wherein the IAP modulator comprises one or more compounds having the formula: 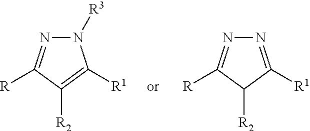
wherein R and R¹ are each independently chosen from:
(i) hydrogen;
(ii) substituted or unsubstituted C₆, C₁₀, or C₁₄ aryl; or
(iii) —C(O)R⁴, wherein R⁴ is a hydrocarbyl unit;
R² is:
(i) hydrogen;
(ii) substituted or unsubstituted C₁-C₄ linear, branched, or cyclic alkyl;
R and R² can be taken together to form a fused ring system having the formula: 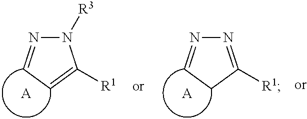
R¹ and R² can be taken together to form a fused ring system having the formula: 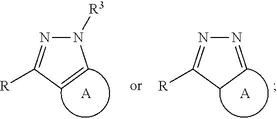
R³ is hydrogen or C₁-C₄ linear alkyl; and
A is one or more substituted or unsubstituted cycloalkyl, aryl, heterocyclic, or heteroaryl rings having from 3 to 14 carbon atoms and from 1 to 5 heteroatoms chosen from oxygen, nitrogen, sulfur, or combinations thereof.

5. The method of claim 4, wherein the compound has the formula: 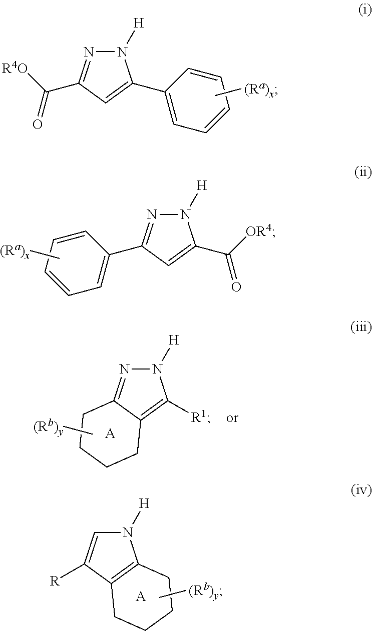
wherein R and R¹ are chosen from:
(i) substituted or unsubstituted C₆, C₁₀, or C₁₄ aryl; or
(ii) —C(O)R⁴;
(iii) wherein R⁴ is chosen from:
(a) substituted or unsubstituted C₁-C₁₀ linear, branched, or cyclic alkyl;
(b) —OR⁵ wherein R⁵ is chosen from:
(i) hydrogen;
(ii) substituted or unsubstituted C₁-C₄ linear or branched alkyl;
each substitution is chosen from:
(i) halogen; and
(ii) —[C(R^{7a})(R^{7b})]_{w}C(O)R⁶;
R⁶ is hydroxy, C₁-C₄ linear or branched alkoxy, or —N(R^{8a})(R^{8b}), each R^{8a} and R^{8b} is independently chosen from hydrogen or C₁-C₁₀ linear, branched or cyclic alkyl;
(iii) —[C(R^{7a})(R^{7b})]_{w}N(R^{9a})(R^{9b});
each R^{9a} and R^{9b} is independently chosen from hydrogen or C₁-C₁₀ linear, branched or cyclic alkyl; or R^{9a} and R^{9b} can be taken together to form a ring having from 3 to 7 atoms;
each R^{7a} and R^{7b} is independently hydrogen or C₁-C₄ linear or branched alkyl; the index w is an integer from 0 to 5;
A is a 6-member aryl, heterocyclic, or heteroaryl ring;
each R^{a} is a substitution for hydrogen, each R^{a} is independently chosen from
(i) C₁-C₁₂ substituted or unsubstituted linear, branched, or cyclic alkyl;
(ii) C₂-C₁₂ substituted or unsubstituted linear, branched, or cyclic alkenyl;
(iii) C₂-C₁₂ substituted or unsubstituted linear or branched alkynyl;
(iv) C₆ or C₁₀ substituted or unsubstituted aryl;
(v) C₁-C₉ substituted or unsubstituted heterocyclic;
(vi) C₁-C₁₁ substituted or unsubstituted heteroaryl;
(vii) —[C(R^{24a})(R^{24b})]ₓOR¹⁰;
R¹⁰ is chosen from:
(a) —H;
(b) C₁-C₁₂ substituted or unsubstituted linear, branched, or cyclic alkyl;
(c) C₆ or C₁₀ substituted or unsubstituted aryl or alkylenearyl;
(d) C₁-C₉ substituted or unsubstituted heterocyclic;
(e) C₁-C₁₁ substituted or unsubstituted heteroaryl;
(viii) —[C(R^{24a})(R^{24b})]ₙN(R^{11a})(R^{11b});
R^{11a} and R^{11b} are each independently chosen from:
(a) —H;
(b) —OR¹²;
R¹² is hydrogen or C₁-C4 linear alkyl;
(c) C₁-C₁₂ substituted or unsubstituted linear, branched, or cyclic alkyl;
(d) C₆ or C₁₀ substituted or unsubstituted aryl;
(e) C₁-C₉ substituted or unsubstituted heterocyclic;
(f) C₁-C₁₁ substituted or unsubstituted heteroaryl; or
(g) R^{11a} and R^{11b} can be taken together to form a substituted or unsubstituted ring having from 3 to 10 carbon atoms and from 0 to 3 heteroatoms chosen from oxygen, nitrogen, and sulfur;
(ix) —[C(R^{24a})(R^{24b})]ₙC(O)R¹³;
R¹³ is
(a) C₁-C₁₂ substituted or unsubstituted linear, branched, or cyclic alkyl;
(b) —OR¹⁴;
R¹⁴ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl;
(c) —N(R^{15a})(R^{15b});
R^{15a} and R^{15b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{15a} and R^{15b} can be taken together to form a substituted or unsubstituted ring having from 3 to 10 carbon atoms and from 0 to 3 heteroatoms chosen from oxygen, nitrogen, and sulfur;
(x) —[C(R^{24a})(R^{24b})]ₙOC(O)R¹⁶;
R¹⁶ is
(a) C₁-C₁₂ substituted or unsubstituted linear, branched, or cyclic alkyl;
(b) —N(R^{17a})(R^{17b});
R^{17a} and R^{17b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{17a} and R^{17b} can be taken together to form a substituted or unsubstituted ring having from 3 to 10 carbon atoms and from 0 to 3 heteroatoms chosen from oxygen, nitrogen, and sulfur;
(xi) —[C)(R^{24a})(R^{24b})]ₙNR¹⁸C(O)R¹⁹;
R¹⁸ is:
(a) —H; or
(b) C₁-C₄ substituted or unsubstituted linear, branched, or cyclic alkyl;
R¹⁹ is:
(a) C₁-C₁₂ substituted or unsubstituted linear, branched, or cyclic alkyl;
(b) —N(R^{20a})(R^{20b});
R^{20a} and R^{20b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{20a} and R^{20b} can be taken together to form a substituted or unsubstituted ring having from 3 to 10 carbon atoms and from 0 to 3 heteroatoms chosen from oxygen, nitrogen, and sulfur;
(xii) —[C(R^{24a})(R^{24b})]ₙCN;
(xiii) —[C(R^{24a})(R^{24b})]ₙNO₂;
(xiv) —[C(R^{24a})(R^{24b})]ₙR²¹;
R²¹ is C₁-C₁₀ linear, branched, or cyclic alkyl substituted by from 1 to 21 halogen atoms chosen from —F, —Cl, —Br, or —I;
(xv) —[C(R^{24a})(R^{24b})]ₙSO₂R²²;
R²² is hydrogen, hydroxyl, substituted or unsubstituted C₁-C₄ linear or branched alkyl; substituted or unsubstituted C₆, C₁₀, or C₁₄ aryl; C₇-C₁₅ alkylenearyl; C₁-C₉ substituted or unsubstituted heterocyclic; or C₁-C₁₁ substituted or unsubstituted heteroaryl;
(xvi) two R^{a} units on the same carbon atom can be taken together to form a unit chosen from ═O, ═S, or ═NR²³;
R²³ is hydrogen, hydroxyl, C₁-C₄ linear or branched alkyl, or C₁-C₄ linear or branched alkoxy;
R^{24a} and R^{24b} are each independently hydrogen or C₁-C₄ alkyl;
the index x is an integer from 0 to 14;
the index n is an integer from 0 to 5;
each R is a substitution for hydrogen independently chosen from
(i) C₁-C₁₂ substituted or unsubstituted linear, branched, or cyclic alkyl;
(ii) C₂-C₁₂ substituted or unsubstituted linear, branched, or cyclic alkenyl;
(iii) C₂-C₁₂ substituted or unsubstituted linear or branched alkynyl;
(iv) C₆ or C₁₀ substituted or unsubstituted aryl;
(v) C₁-C₉ substituted or unsubstituted heterocyclic;
(vi) C₁-C₁₁ substituted or unsubstituted heteroaryl;
(vii) —[C(R^{39a})(R^{39b})]ₘOR²;
R²⁵ is chosen from:
(a) —H;
(b) C₁-C₁₂ substituted or unsubstituted linear, branched, or cyclic alkyl;
(c) C₆ or C₁₀ substituted or unsubstituted aryl or alkylenearyl;
(d) C₁-C₉ substituted or unsubstituted heterocyclic;
(e) C₁-C₁₁ substituted or unsubstituted heteroaryl;
(viii) —[C(R^{39a})(R^{39b})]ₘN(R^{26a})(R^{26b});
R^{26a} and R^{26b} are each independently chosen from:
(a) —H;
(b) —OR²⁷;
R²⁷ is hydrogen or C₁-C4 linear alkyl;
(c) C₁-C₁₂ substituted or unsubstituted linear, branched, or cyclic alkyl;
(d) C₆ or C₁₀ substituted or unsubstituted aryl;
(e) C₁-C₉ substituted or unsubstituted heterocyclic;
(f) C₁-C₁₁ substituted or unsubstituted heteroaryl; or
(g) R^{26a} and R^{26b} can be taken together to form a substituted or unsubstituted ring having from 3 to 10 carbon atoms and from 0 to 3 heteroatoms chosen from oxygen, nitrogen, and sulfur;
(ix) —[C(R^{39a})(R^{39b})]ₘC(O)R²⁸;
R²⁸ is
(a) C₁-C₁₂ substituted or unsubstituted linear, branched, or cyclic alkyl;
(b) —OR²⁹;
R²⁹ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl;
(c) —N(R^{30a})(R^{30b});
R^{30a} and R^{30b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{30a} and R^{30b} can be taken together to form a substituted or unsubstituted ring having from 3 to 10 carbon atoms and from 0 to 3 heteroatoms chosen from oxygen, nitrogen, and sulfur;
(x) —[C(R^{39a})(R^{39b})]ₘOC(O)R³¹;
R³¹ is
(a) C₁-C₁₂ substituted or unsubstituted linear, branched, or cyclic alkyl;
(b) —N(R^{32a})(R^{32b});
R^{32a} and R^{32b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{32a} and R^{32b} can be taken together to form a substituted or unsubstituted ring having from 3 to 10 carbon atoms and from 0 to 3 heteroatoms chosen from oxygen, nitrogen, and sulfur;
(xi) —[C(R^{39a})(R^{39b})]ₘNR³³C(O)R³⁴;
R³³ is:
(a) —H; or
(b) C₁-C₄ substituted or unsubstituted linear, branched, or cyclic alkyl;
R³⁴ is:
(a) C₁-C₁₂ substituted or unsubstituted linear, branched, or cyclic alkyl;
(b) —N(R^{35a})(R^{35b});
R^{35a} and R^{35b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{35a} and R^{35b} can be taken together to form a substituted or unsubstituted ring having from 3 to 10 carbon atoms and from 0 to 3 heteroatoms chosen from oxygen, nitrogen, and sulfur;
(xii) —[C(R^{39a})(R^{39b})]ₘCN;
(xiii) —[C(R^{39a})(R^{39b})]ₘNO₂;
(xiv) —[C(R^{39a})(R^{39b})]ₘR³⁶;
R³⁶ is C₁-C₁₀ linear, branched, or cyclic alkyl substituted by from 1 to 21 halogen atoms chosen from —F, —Cl, —Br, or —I;
(xv) —[C(R^{39a})(R^{39b})]ₘSO₂R³⁷;
R³⁷ is hydrogen, hydroxyl, substituted or unsubstituted C₁-C₄ linear or branched alkyl; substituted or unsubstituted C₆, C₁₀, or C₁₄ aryl; C₇-C₁₅ alkylenearyl; C₁-C₉ substituted or unsubstituted heterocyclic; or C₁-C₁₁ substituted or unsubstituted heteroaryl;
(xvi) two R^{b} units on the same carbon atom can be taken together to form a unit chosen from ═O, ═S, or ═NR³⁸;
R³⁸ is hydrogen, hydroxyl, C₁-C₄ linear or branched alkyl, or C₁-C₄ linear or branched alkoxy;
R^{39a} and R^{39b} are each independently hydrogen or C₁-C₄ alkyl;
the index y is an integer from 0 to 14; and
the index m is an integer from 0 to 5.

6. The method of claim 5, wherein the substitutes for hydrogen on R^{a} and R^{b} substitutions for hydrogen, are organic radicals each independently chosen from:
(i) C₁-C₁₂ linear, branched, or cyclic alkyl, alkenyl, and alkynyl;
(ii) substituted or unsubstituted C₆ or C₁₀ aryl;
(iii) substituted or unsubstituted C₆ or C₁₀ alkylenearyl;
(iv) substituted or unsubstituted C₁-C₉ heterocyclic rings;
(v) substituted or unsubstituted C₁-C₉ heteroaryl rings;
(vi) —(CR^{102a}R^{102b})_{z}OR¹⁰¹;
(vii) —(CR^{102a}R^{102b})_{z}C(O)R¹⁰¹;
(viii) —(CR^{102a}R^{102b})_{z}C(O)OR¹⁰¹;
(ix) —(CR^{102a}R^{102b})_{z}C(O)N(R¹⁰¹)₂;
(x) —(CR^{102a}R^{102b})_{z}N(R¹⁰¹)₂;
(xi) halogen;
(xii) —(CR^{102a}R^{102b})_{z}CN;
(xiii) —(CR^{102a}R^{102b})_{z}NO₂;
(xiv) —CHⱼXₖ; wherein X is halogen, the index j is an integer from 0 to 2, j+k 3;
(xv) —(CR^{102a}R^{102b})_{z}SR¹⁰¹;
(xvi) —(CR^{102a}R^{102b})_{z}SO₂R¹⁰¹; and
(xvii) —(CR^{102a}R^{102b})_{z}SO₃R¹⁰¹;
wherein each R¹⁰¹ is independently hydrogen, substituted or unsubstituted C₁-C₄ linear, branched, or cyclic alkyl, phenyl, benzyl, heterocyclic, or heteroaryl; or two R¹⁰¹ units can be taken together to form a ring comprising 3-7 atoms; R^{102a} and R^{102b} are each independently hydrogen or C₁-C₄ linear or branched alkyl; the index z is from 0 to 4.

7. The method of claim 4, wherein the compound has the formula: 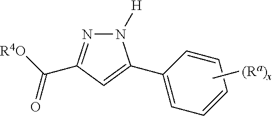
wherein R⁴ is chosen from:
(i) hydrogen;
(ii) C₁-C₄ linear or branched alkyl; or
(iii) —[CH₂]_{w}C(O)N(R^{8a})(R^{8b}); and
each R^{a} is chosen from:
(i) C₁-C₄ linear or branched alkyl;
(ii) C₁-C₄ linear or branched alkoxy;
(iii) —OH;
(iv) —F;
(v) —Cl;
(vi) —Br;
(vii) —NO₂;
(viii) —NH₂; and
(ix) —CF₃;
the index w is an integer from 0 to 3; and the index x is an integer from 0 to 5.

8. The method of claim 4, wherein the compound has the formula: 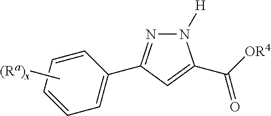
wherein R⁴ is chosen from:
(i) hydrogen;
(ii) C₁-C₄ linear or branched alkyl; or
(iii) —[CH₂]_{w}C(O)N(R^{8a})(R^{8b}); and
each R^{a} is chosen from:
(i) C₁-C₄ linear or branched alkyl;
(ii) C₁-C₄ linear or branched alkoxy;
(iii) —OH;
(iv) —F;
(v) —Cl;
(vi) —Br;
(vii) —NO₂;
(viii) —NH₂; and
(ix) —CF₃;
the index w is an integer from 0 to 3; and the index x is an integer from 0 to 5.

9. The method of claim 4, wherein the compound has the formula: 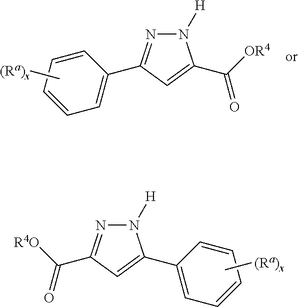
wherein two adjacent R^{a} units are taken together to form a substituted or unsubstituted fused ring chosen from:
(i) cycloalkyl;
(ii) aryl;
(iii) heterocyclic; or
(iv) heteroaryl;
the fused ring having from 6 to 12 carbon atoms, from 0 to 4 heteroatoms chosen from oxygen, nitrogen, and sulfur; and
the index x is an integer from 0 to 5.

10. The method of claim 9, wherein the fused ring has from 1 to 14 substitutions for hydrogen each independently chosen from:
(i) C₁-C₁₂ linear, branched, or cyclic alkyl, alkenyl, and alkynyl;
(ii) substituted or unsubstituted C₆ or C₁₀ aryl;
(iii) substituted or unsubstituted C₆ or C₁₀ alkylenearyl;
(iv) substituted or unsubstituted C₁-C₉ heterocyclic rings;
(v) substituted or unsubstituted C₁-C₉ heteroaryl rings;
(vi) —(CR^{102a}R^{102b})_{z}OR¹⁰¹;
(vii) —(CR^{102a}R^{102b})_{z}C(O)R¹⁰¹;
(viii) —(CR^{102a}R^{102b})_{z}C(O)OR¹⁰¹;
(ix) —(CR^{102a}R^{102b})_{z}C(O)N(R¹⁰¹)₂;
(x) halogen;
(xi) —(CR^{102a}R^{102b})_{z}CN;
(xii) —(CR^{102a}R^{102b})_{z}NO₂;
(xiii) —CHⱼXₖ; wherein X is halogen, the index j is an integer from 0 to 2, j+k 3;
(xiv) —(CR^{102a}R^{102b})_{z}SR¹⁰¹;
(xv) —(CR^{102a}R^{102b})_{z}SO₂R¹⁰¹; and
(xvi) —(CR^{102a}R^{102b})_{z}SO₃R¹⁰¹;
wherein each R¹⁰¹ is independently hydrogen, substituted or unsubstituted C₁-C₄ linear, branched, or cyclic alkyl, phenyl, benzyl, heterocyclic, or heteroaryl; or two R¹⁰¹ units can be taken together to form a ring comprising 3-7 atoms; R^{102a} and R^{102b} are each independently hydrogen or C₁-C₄ linear or branched alkyl; the index z is from 0 to 4.

11. The method of claim 4, wherein the compound has the formula: 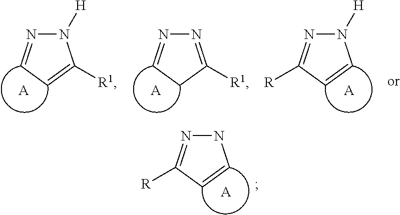
wherein R and R¹ have the formula —C(O)R⁴; wherein R⁴ is chosen from:
(a) substituted or unsubstituted C₁-C₁₀ linear, branched, or cyclic alkyl;
(b) —OR⁵ wherein R⁵ is chosen from:
(i) hydrogen;
(ii) substituted or unsubstituted C₁-C₄ linear or branched alkyl;
each substitution is chosen from:
(a) halogen; and
(b) —[C(R^{7a})(R^{7b})]_{w}C(O)R⁶;
R⁶ is hydroxy, C₁-C₄ linear or branched alkoxy, or —N(R^{8a})(R^{8b}), each R^{8a} and R^{8b} is independently chosen from hydrogen or C₁-C₁₀ linear, branched or cyclic alkyl;
(c) —[C(R^{7a})(R^{7b})]_{w}N(R^{9a})(R^{9b});
each R^{9a} and R^{9b} is independently chosen from hydrogen or C₁-C₁₀ linear, branched or cyclic alkyl; or R^{9a} and R^{9b} can be taken together to form a ring having from 3 to 7 atoms; and
each R^{7a} and R^{7b} is independently hydrogen or C₁-C₄ linear or branched alkyl; and the index w is an integer from 0 to 5.

12. The method of claim 4, wherein the compound has the formula:
(i) 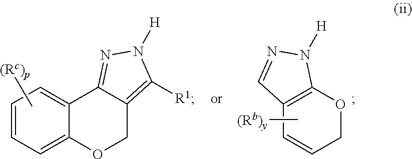
wherein each R^{b} is a substitution for hydrogen independently chosen from
(i) C₁-C₁₂ substituted or unsubstituted linear, branched, or cyclic alkyl;
(ii) C₂-C₁₂ substituted or unsubstituted linear, branched, or cyclic alkenyl;
(iii) C₂-C₁₂ substituted or unsubstituted linear or branched alkynyl;
(iv) C₆ or C₁₀ substituted or unsubstituted aryl;
(v) C₁-C₉ substituted or unsubstituted heterocyclic;
(vi) C₁-C₁₁ substituted or unsubstituted heteroaryl;
(vii) —[C(R^{39a})(R^{39b})]ₘOR²⁵;
R²⁵ is chosen from:
(a) —H;
(b) C₁-C₁₂ substituted or unsubstituted linear, branched, or cyclic alkyl;
(c) C₆ or C₁₀ substituted or unsubstituted aryl or alkylenearyl;
(d) C₁-C₉ substituted or unsubstituted heterocyclic;
(e) C₁-C₁₁ substituted or unsubstituted heteroaryl;
(viii) —[C(R^{39a})(R^{39b})]ₘN(R^{26a})(R^{26b});
R^{26a} and R^{26b} are each independently chosen from:
(a) —H;
(b) —OR²⁷;
R²⁷ is hydrogen or C₁-C₄ linear alkyl;
(c) C₁-C₁₂ substituted or unsubstituted linear, branched, or cyclic alkyl;
(d) C₆ or C₁₀ substituted or unsubstituted aryl;
(e) C₁-C₉ substituted or unsubstituted heterocyclic;
(f) C₁-C₁₁ substituted or unsubstituted heteroaryl; or
(g) R^{26a} and R^{26b} can be taken together to form a substituted or unsubstituted ring having from 3 to 10 carbon atoms and from 0 to 3 heteroatoms chosen from oxygen, nitrogen, and sulfur;
(ix) —[C(R^{39a})(R^{39b})]ₘC(O)R²⁸;
R²⁸ is
(a) C₁-C₁₂ substituted or unsubstituted linear, branched, or cyclic alkyl;
(b) —OR²⁹;
R²⁹ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl;
(c) —N(R^{30a})(R^{30b});
R^{30a} and R^{30b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{30a} and R^{30b} can be taken together to form a substituted or unsubstituted ring having from 3 to 10 carbon atoms and from 0 to 3 heteroatoms chosen from oxygen, nitrogen, and sulfur;
(x) —[C(R^{39a}) (R^{39b})]ₘOC(O)R³¹;
R³¹ is
(a) C₁-C₁₂ substituted or unsubstituted linear, branched, or cyclic alkyl;
(b) —N(R^{32a})(R^{32b});
R^{32a} and R^{32b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{32a} and R^{32b} can be taken together to form a substituted or unsubstituted ring having from 3 to 10 carbon atoms and from 0 to 3 heteroatoms chosen from oxygen, nitrogen, and sulfur;
(xi) —[C(R^{39a})(R^{39b})]ₘNR³³C(O)R³⁴;
R³³ is:
(a) —H; or
(b) C₁-C₄ substituted or unsubstituted linear, branched, or cyclic alkyl;
R³⁴ is
(a) C₁-C₁₂ substituted or unsubstituted linear, branched, or cyclic alkyl;
(b) —N(R^{35a})(R^{35b});
R^{35a} and R^{35b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{35a} and R^{35b} can be taken together to form a substituted or unsubstituted ring having from 3 to 10 carbon atoms and from 0 to 3 heteroatoms chosen from oxygen, nitrogen, and sulfur;
(xii) —[C(R^{39a})(R^{39b})]ₘCN;
(xiii) —[C(R^{39a})(R^{39b})]ₘNO₂;
(xiv) —[C(R^{39a})(R^{39b})]ₘR³⁶;
R³⁶ is C₁-C₁₀ linear, branched, or cyclic alkyl substituted by from 1 to 21 halogen atoms chosen from —F, —Cl, —Br, or —I;
(xv) —[C(R^{39a})(R^{39b})]ₘSO₂R³⁷;
R³⁷ is hydrogen, hydroxyl, substituted or unsubstituted C₁-C₄ linear or branched alkyl; substituted or unsubstituted C₆, C₁₀, or C₁₄ aryl; C₇-C₁₅ alkylenearyl; C₁-C₉ substituted or unsubstituted heterocyclic; or C₁-C₁₁ substituted or unsubstituted heteroaryl;
(xvi) two R^{b} units on the same carbon atom can be taken together to form a unit chosen from ═O, ═S, or ═NR³⁸;
R³⁸ is hydrogen, hydroxyl, C₁-C₄ linear or branched alkyl, or C₁-C₄ linear or branched alkoxy;
R^{39a} and R^{39b} are each independently hydrogen or C₁-C₄ alkyl;
the index y is an integer from 0 to 14; and
the index m is an integer from 0 to 5;
each R^{c} is independently chosen from:
(i) C₁-C₁₂ substituted or unsubstituted linear, branched, or cyclic alkyl;
(ii) C₂-C₁₂ substituted or unsubstituted linear, branched, or cyclic alkenyl;
(iii) C₂-C₁₂ substituted or unsubstituted linear or branched alkynyl;
(iv) C₆ or C₁₀ substituted or unsubstituted aryl;
(v) C₁-C₉ substituted or unsubstituted heterocyclic;
(vi) C₁-C₁₁ substituted or unsubstituted heteroaryl;
(vii) —[C(R^{54a})(R^{54b})]_{q}OR⁴⁰;
R⁴⁰ is chosen from:
(a) —H;
(b) C₁-C₁₂ substituted or unsubstituted linear, branched, or cyclic alkyl;
(c) C₆ or C₁₀ substituted or unsubstituted aryl or alkylenearyl;
(d) C₁-C₉ substituted or unsubstituted heterocyclic;
(e) C₁-C₁₁ substituted or unsubstituted heteroaryl;
(viii) —[C(R^{54a})(R^{54b})]_{q}N(R^{41a})(R^{41b});
R^{41a} and R^{41b} are each independently chosen from:
(a) —H;
(b) —OR⁴²;
R⁴² is hydrogen or C₁-C4 linear alkyl;
(c) C₁-C₁₂ substituted or unsubstituted linear, branched, or cyclic alkyl;
(d) C₆ or C₁₀ substituted or unsubstituted aryl;
(e) C₁-C₉ substituted or unsubstituted heterocyclic;
(f) C₁-C₁₁ substituted or unsubstituted heteroaryl; or
(g) R^{41a} and R^{41b} can be taken together to form a substituted or unsubstituted ring having from 3 to 10 carbon atoms and from 0 to 3 heteroatoms chosen from oxygen, nitrogen, and sulfur;
(ix) —[C(R^{54a})(R^{54b})]_{q}C(O)R⁴³;
R⁴³ is
(a) C₁-C₁₂ substituted or unsubstituted linear, branched, or cyclic alkyl;
(b) —OR⁴⁴;
R⁴⁴ is hydrogen, substituted or unsubstituted C₁-C₄ linear alkyl, C₆ or C₁₀ substituted or unsubstituted aryl, C₁-C₉ substituted or unsubstituted heterocyclic, C₁-C₁₁ substituted or unsubstituted heteroaryl;
(c) —N(R^{45a})(R^{45b});
R^{45a} and R^{45b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{45a} and R^{45b} can be taken together to form a substituted or unsubstituted ring having from 3 to 10 carbon atoms and from 0 to 3 heteroatoms chosen from oxygen, nitrogen, and sulfur;
(x) —[C(R^{54a})(R^{54b})]_{q}OC(O)R⁴⁶;
R⁴⁶ is
(a) C₁-C₁₂ substituted or unsubstituted linear, branched, or cyclic alkyl;
(b) —N(R^{47a})(R^{47b});
R^{47a} and R^{47b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R^{47a} and R^{7b} can be taken together to form a substituted or unsubstituted ring having from 3 to 10 carbon atoms and from 0 to 3 heteroatoms chosen from oxygen, nitrogen, and sulfur;
(xi) —[C)(R^{54a})(R^{54b})]_{q}NR⁴⁸C(O)R⁴⁹;
R⁴⁸ is:
(a) —H; or
(b) C₁-C₄ substituted or unsubstituted linear, branched, or cyclic alkyl;
R⁴⁹ is
(a) C₁-C₁₂ substituted or unsubstituted linear, branched, or cyclic alkyl;
(b) —N(R^{50a})(R^{50b});
R^{50a} and R^{50b} are each independently hydrogen, C₁-C₁₂ substituted or unsubstituted linear, branched, or cyclic alkyl; C₆ or C₁₀ substituted or unsubstituted aryl; C₁-C₉ substituted or unsubstituted heterocyclic; C₁-C₁₁ substituted or unsubstituted heteroaryl; or R⁵⁰a and R^{50b} can be taken together to form a substituted or unsubstituted ring having from 3 to 10 carbon atoms and from 0 to 3 heteroatoms chosen from oxygen, nitrogen, and sulfur;
(xii) —[C(R^{54a})(R^{54b})]_{q}CN;
(xiii) —[C(R^{54a})(R^{54b})]_{q}NO₂;
(xiv) —[C(R^{54a})(R^{54b})]_{q}R⁵¹;
R⁵¹ is C₁-C₁₀ linear, branched, or cyclic alkyl substituted by from 1 to 21 halogen atoms chosen from —F, —Cl, —Br, or —I;
(xv) —[C(R^{54a})(R^{54b})]_{q}SO₂R⁵²;
R⁵² is hydrogen, hydroxyl, substituted or unsubstituted C₁-C₄ linear or branched alkyl; substituted or unsubstituted C₆, C₁₀, or C₁₄ aryl; C₇-C₁₅ alkylenearyl; C₁-C₉ substituted or unsubstituted heterocyclic; or C₁-C₁₁ substituted or unsubstituted heteroaryl;
(xvi) two R^{b} units on the same carbon atom can be taken together to form a unit chosen from ═O, ═S, or ═NR⁵³;
R⁵³ is hydrogen, hydroxyl, C₁-C₄ linear or branched alkyl, or C₁-C₄ linear or branched alkoxy;
R^{54a} and R^{54b} are each independently hydrogen or C₁-C₄ alkyl;
the index p is an integer from 0 to 14; and
the index q is an integer from 0 to 5.

13. The method of claim 4, wherein the compound has the formula:

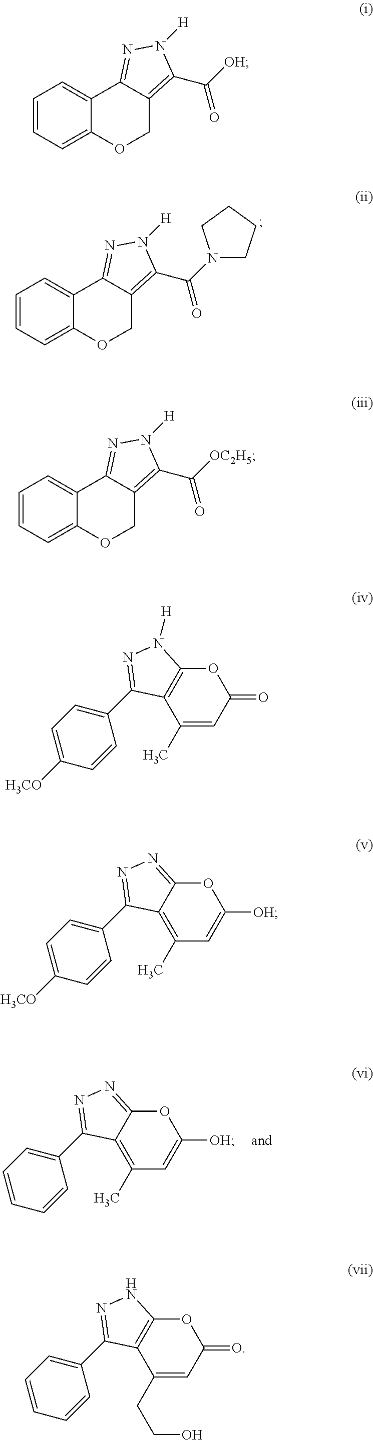

14. The method of claim 4, wherein the compound has the formula: 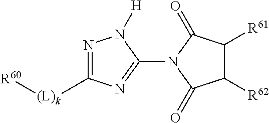
wherein R⁶⁰ is chosen from:
(i) hydrogen;
(ii) substituted or unsubstituted C₆ or C₁₀ aryl;
(iii) substituted or unsubstituted C₁-C₉ heteroaryl; or
(iv) substituted or unsubstituted C₁-C₉ heterocyclic;
R⁶¹ and R⁶² are taken together to form a ring chosen from:
(i) saturated or unsaturated cycloalkyl;
(ii) saturated or unsaturated bicycloalkyl; or
(iii) aryl;
L is a linking unit having from 1 to 5 carbon atoms; and
the index k is 0 or 1.

15. The method of claim 14, wherein the compound has the formula:

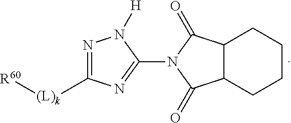

16. The method of claim 15, wherein R⁶⁰ is phenyl.

17. The method of claim 15, wherein R⁶⁰ is a substituted or unsubstituted C₁, C₂, C₃, or C₄ heteroaryl or heterocyclic 5-member ring having a formula chosen from: 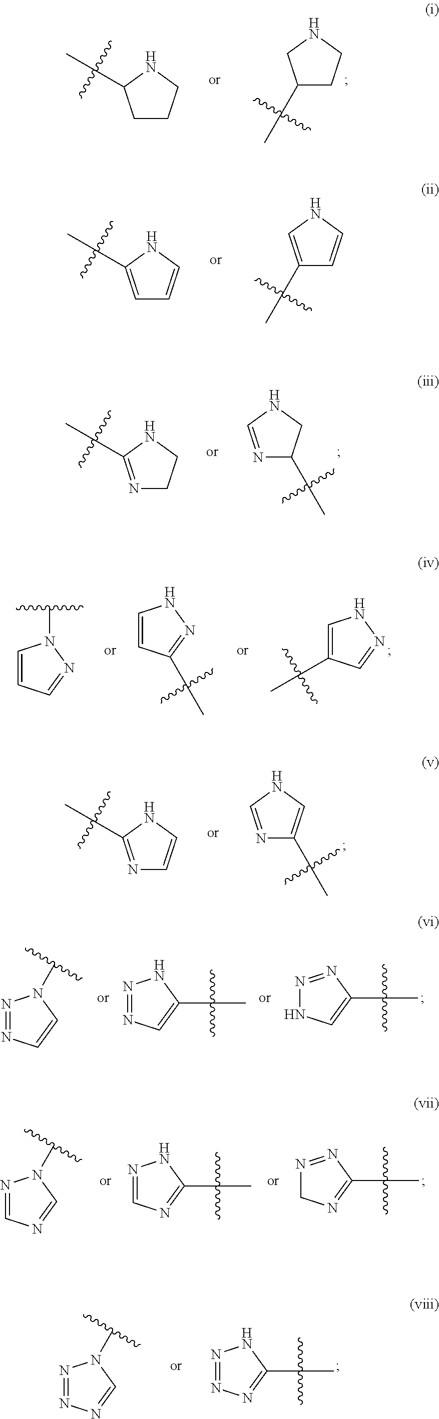 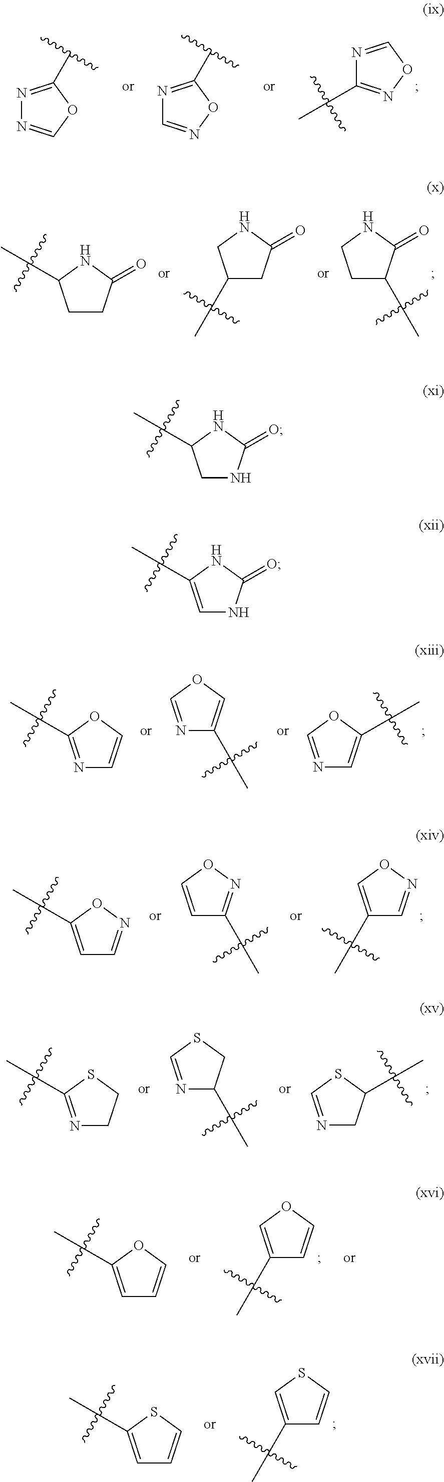
wherein any of the ring hydrogen atoms can be substituted by a hydrocarbyl unit.

18. The method of claim 17, wherein R⁶⁰ is a substituted or unsubstituted C₁, C₂, C₃, or C₄ heteroaryl 5-member ring having a formula chosen from:

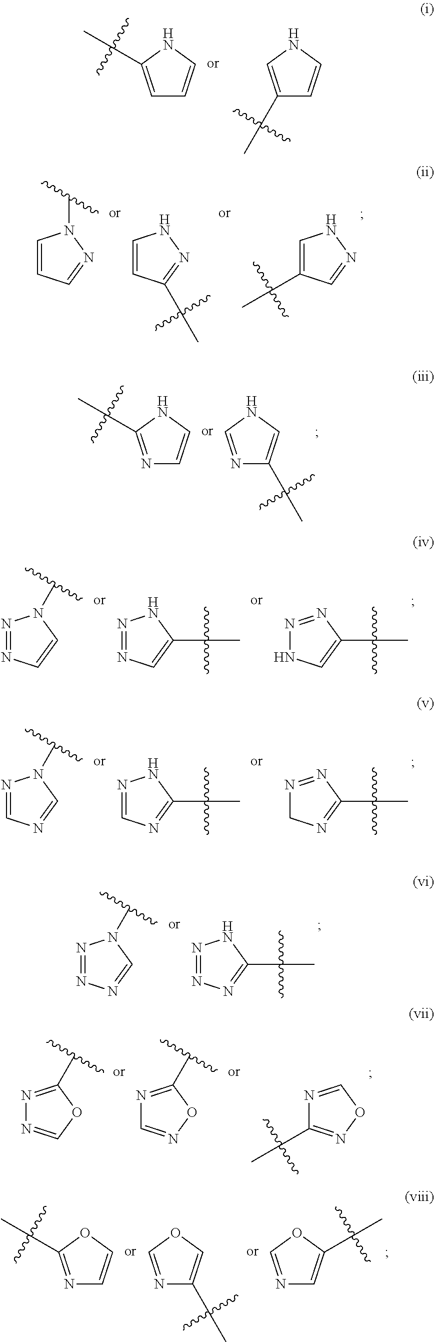

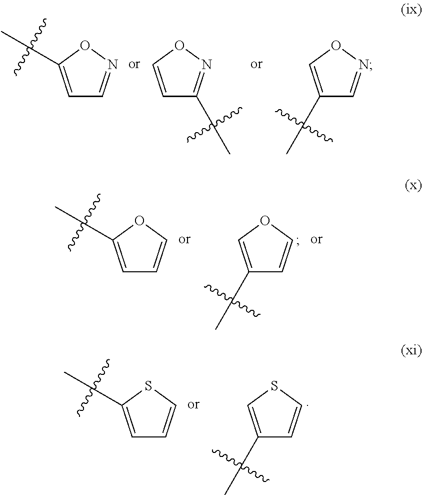

19. The method of claim 18, wherein R⁶⁰ has the formula:

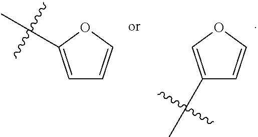

20. The method of claim 15, wherein R⁶⁰ is a substituted or unsubstituted C₃, C₄, or C₅ heteroaryl or heterocyclic 6-member ring having a formula chosen from: 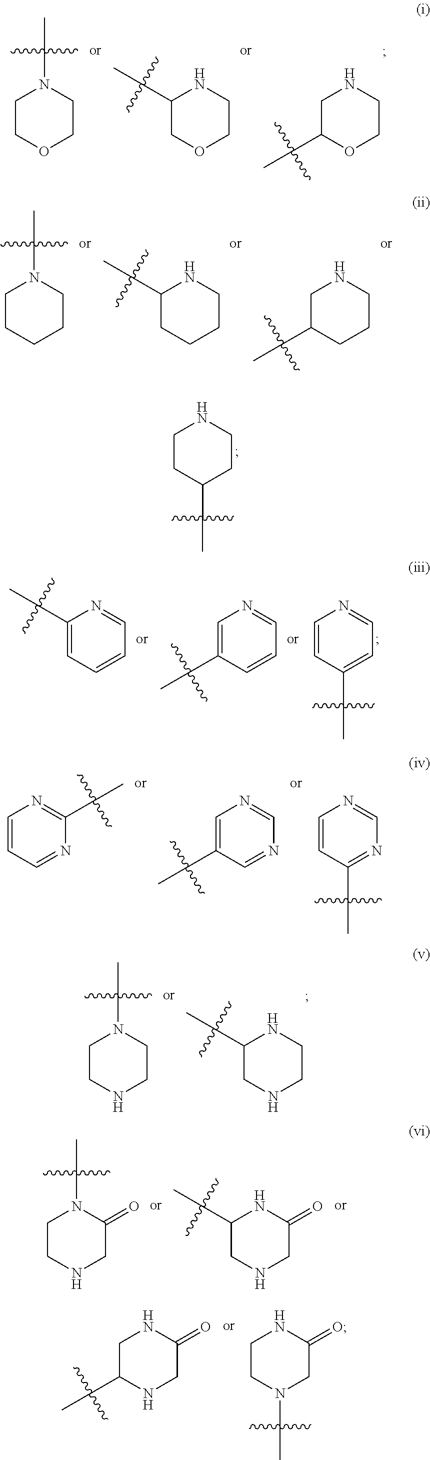 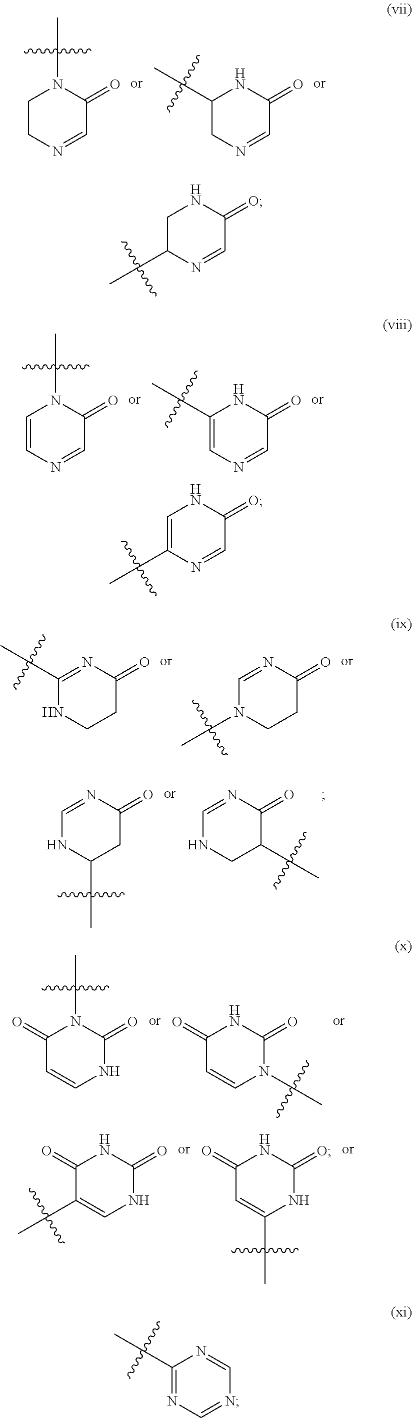
wherein any of the ring hydrogen atoms can be substituted by a hydrocarbyl unit.

21. The method of claim 15, wherein R⁶⁰ is a substituted or unsubstituted C₃, C₄, or C₅ heteroaryl 6-member ring having a formula chosen from:

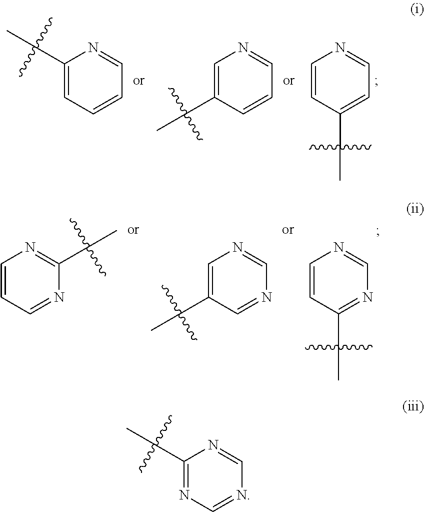

22. The method of claim 15, wherein R⁶⁰ has the formula:

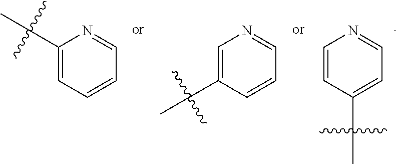

23. The method of claim 15, wherein R⁶⁰ is a substituted or unsubstituted C₇ or C₈ heteroaryl or heterocyclic fused having a formula chosen from: 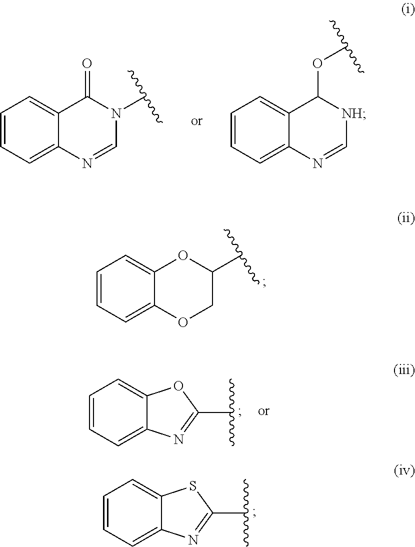
wherein any of the ring hydrogen atoms can be substituted by a hydrocarbyl unit.

24. The method of claim 15, wherein L is chosen from:
(i) —CH₂—;
(ii) —CH₂CH₂—;
(iii) —CH₂CH₂CH₂—;
(iv) —CH₂CH₂CH₂CH₂—;
(v) —CH₂CH(CH₃)CH₂—; or
(vi) —CH₂CH(CH₃)CH₂CH₂—.

25. The method of claim 15, wherein L is —CH₂— or —CH₂CH₂—.

26. The method of claim 15, wherein the index k is 0.

27. The method of claim 14, wherein the compound has the formula:

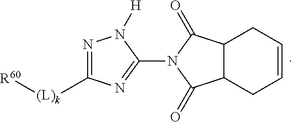

28. The method of claim 27, wherein R⁶⁰ is phenyl.

29. The method of claim 27, wherein R⁶⁰ is a substituted or unsubstituted C₁, C₂, C₃, or C₄ heteroaryl or heterocyclic 5-member ring having a formula chosen from: 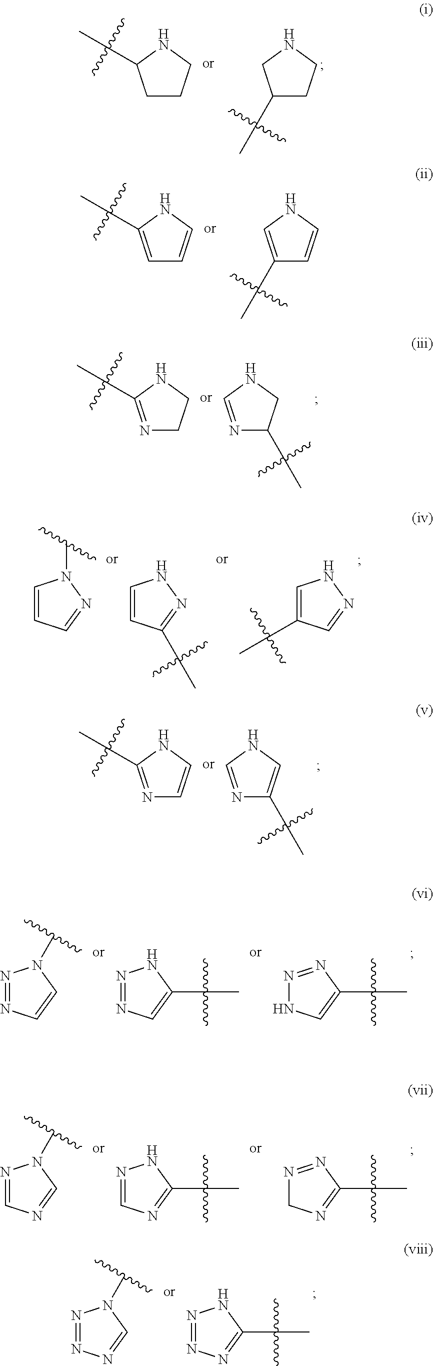 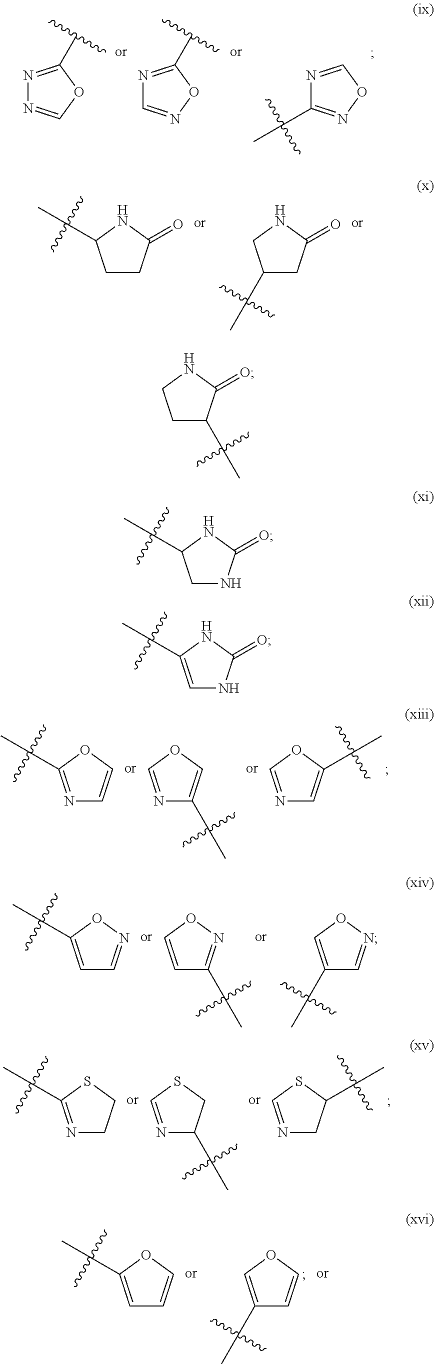 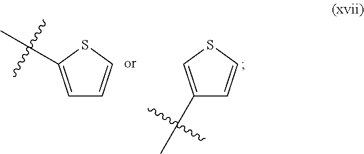
wherein any of the ring hydrogen atoms can be substituted by a hydrocarbyl unit.

30. The method of claim 29, wherein R⁶⁰ is a substituted or unsubstituted C₁, C₂, C₃, or C₄ heteroaryl 5-member ring having a formula chosen from:

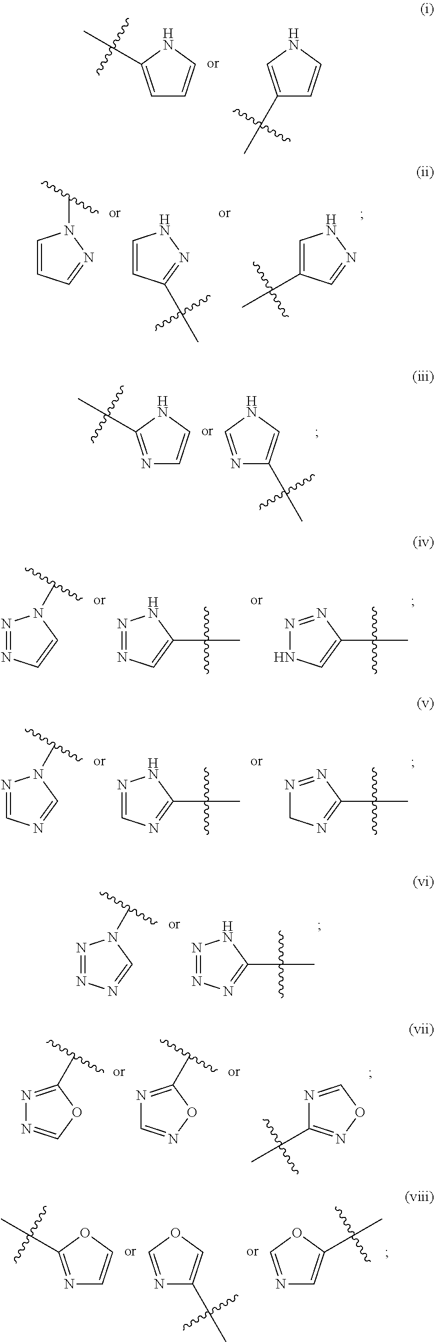

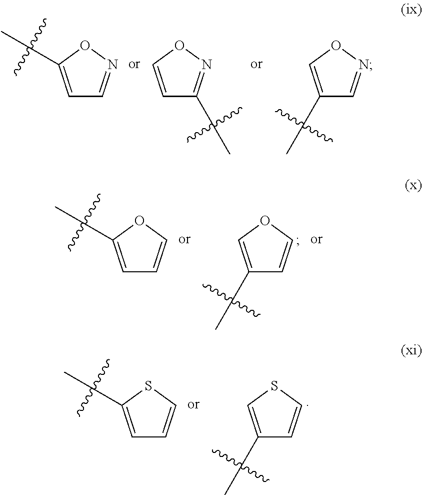

31. The method of claim 30, wherein R⁶⁰ has the formula:

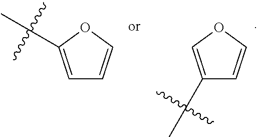

32. The method of claim 27, wherein R⁶⁰ is a substituted or unsubstituted C₃, C₄, or C₅ heteroaryl or heterocyclic 6-member ring having a formula chosen from: 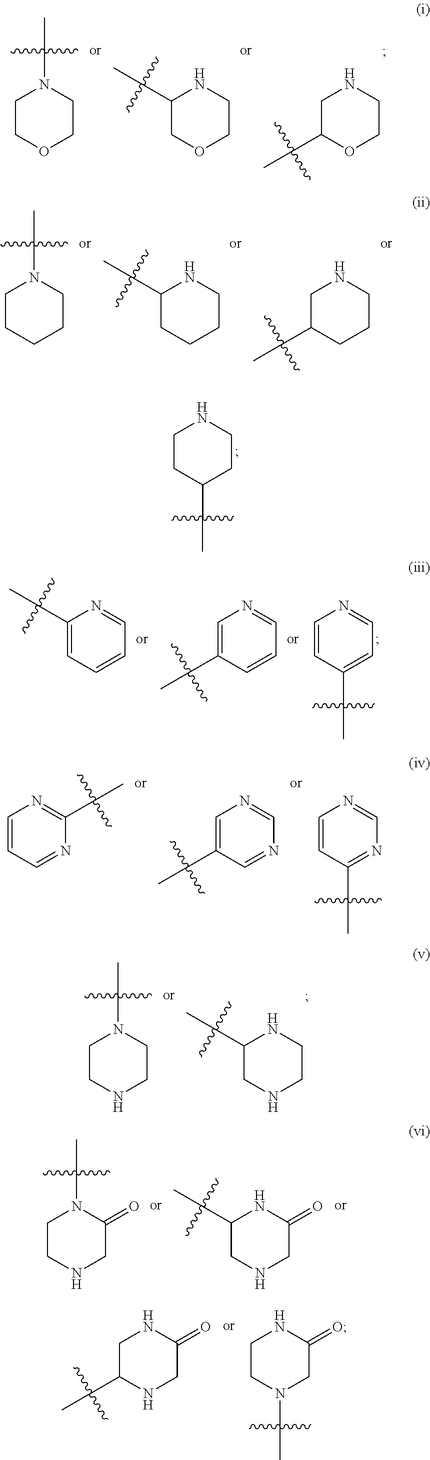 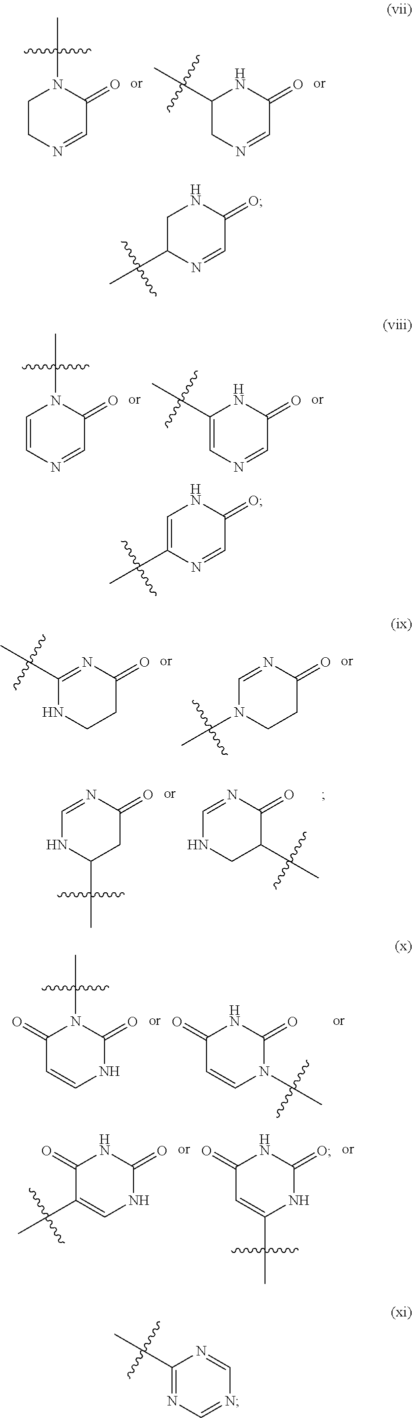
wherein any of the ring hydrogen atoms can be substituted by a hydrocarbyl unit.

33. The method of claim 27, wherein R⁶⁰ is a substituted or unsubstituted C₃, C₄, or C₅ heteroaryl or heterocyclic 6-member ring having a formula chosen from:

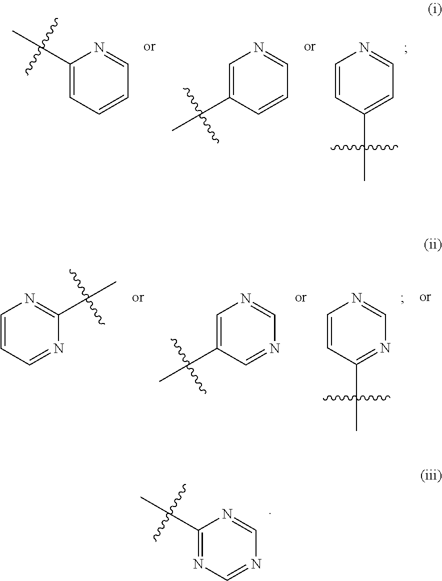

34. The method of claim 33, wherein R⁶⁰ has the formula:

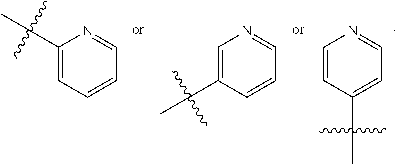

35. The method of claim 27, wherein R⁶⁰ is a substituted or unsubstituted C₇ or C₈ heteroaryl or heterocyclic fused having a formula chosen from: 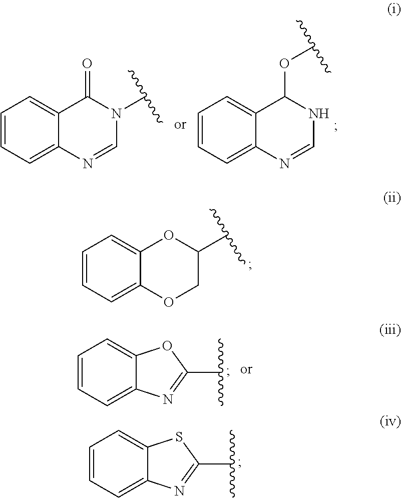
wherein any of the ring hydrogen atoms can be substituted by a hydrocarbyl unit.

36. The method of claim 27, wherein L is chosen from:
(i) —CH₂—;
(ii) —CH₂CH₂—;
(iii) —CH₂CH₂CH₂—;
(iv) —CH₂CH₂CH₂CH₂—;
(v) —CH₂CH(CH₃)CH₂—; or
(vi) —CH₂CH(CH₃)CH₂CH₂—.

37. The method of claim 27, wherein L is —CH₂— or —CH₂CH₂—.

38. The method of claim 27, wherein the index k is 0.

39. The method of claim 4, wherein the compound has the formula: 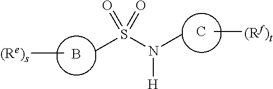
wherein B and C are a ring independently chosen from:
(i) C₆ or C₁₀ aryl; or
(ii) C₁-C₉ heteroaryl;
R^{e} and R^{f} are from 1 to 9 substitutions for hydrogen, each R^{e} and R^{f} is independently chosen from:
(i) substituted or unsubstituted C₁-C₁₀ linear, branched or cyclic alkyl;
(ii) substituted or unsubstituted C₂-C₁₀ linear, branched or cyclic alkenyl;
(iii) substituted or unsubstituted C₂-C₁₀ linear or branched or alkynyl;
(iv) substituted or unsubstituted C₁-C₁₀ linear, branched or cyclic alkoxy;
(v) substituted or unsubstituted C₂-C₁₀ linear, branched or cyclic alkenoxy;
(vi) substituted or unsubstituted C₂-C₁₀ linear or branched alkynoxy; or
(vii) halogen;
the index s is an integer from 0 to 9; and the index t is an integer from 0 to 9.

40. The method of claim 39, wherein B is substituted or unsubstituted C₆ or C₁₀ aryl.

41. The method of claim 39, wherein B is C₆ aryl.

42. The method of claim 39, wherein B is substituted or unsubstituted C₁-C₉ heteroaryl.

43. The method of claim 39, wherein B is substituted or unsubstituted C₁, C₂, C₃, or C₄ heteroaryl 5-member ring having a formula chosen from:

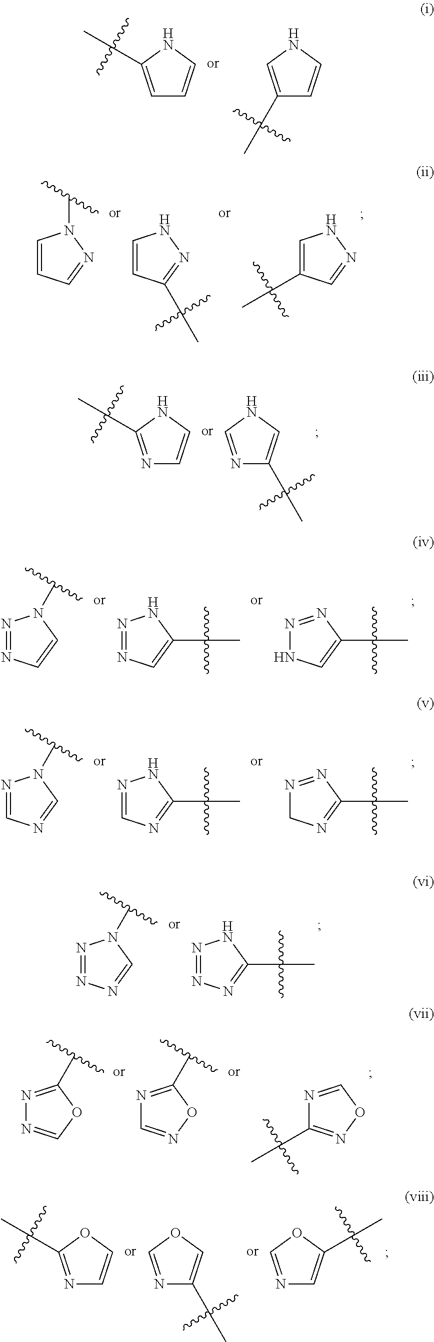

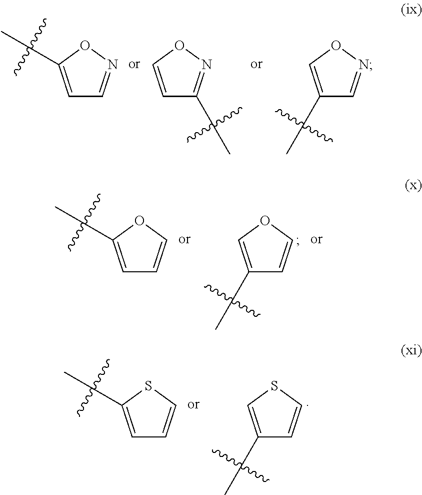

44. The method of claim 39, wherein B is a C₃, C₄, or C₅ heteroaryl 6-member ring having a formula chosen from:

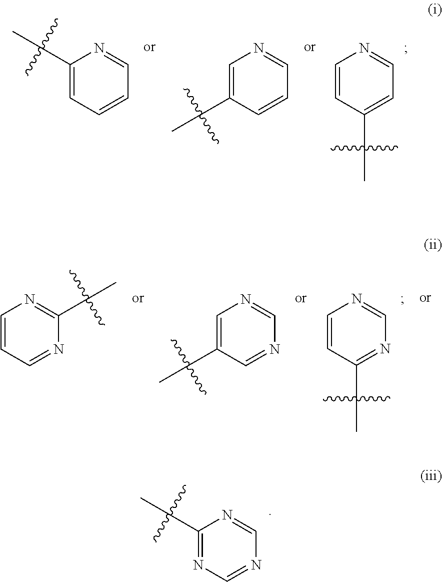

45. The method of claim 39, wherein B is substituted or unsubstituted C₆ or C₁₀ aryl.

46. The method of claim 39, wherein C is C₆ aryl.

47. The method of claim 39, wherein C is substituted or unsubstituted C₁-C₉ heteroaryl.

48. The method of claim 39, wherein C is substituted or unsubstituted C₁, C₂, C₃, or C₄ heteroaryl 5-member ring having a formula chosen from:

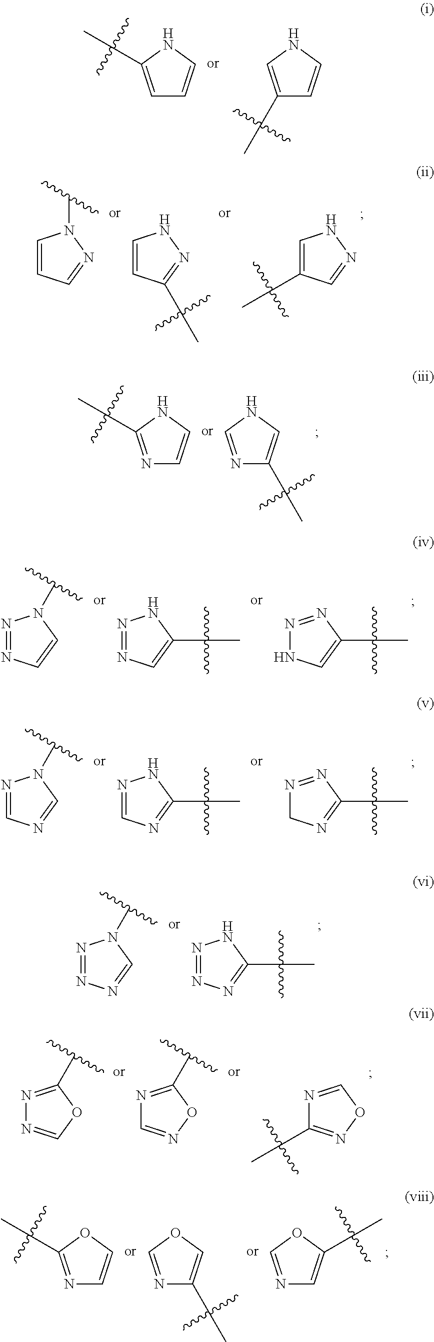

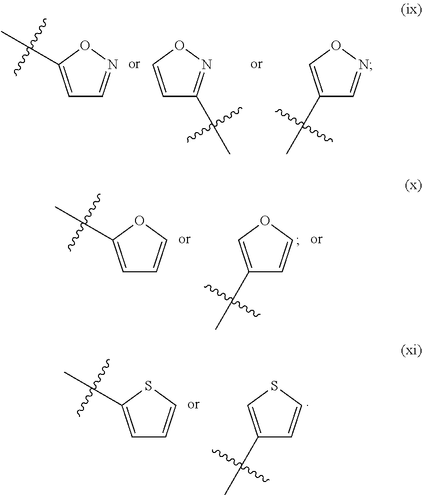

49. The method of claim 39, wherein C is a C₃, C₄, or C₅ heteroaryl 6-member ring having a formula chosen from:

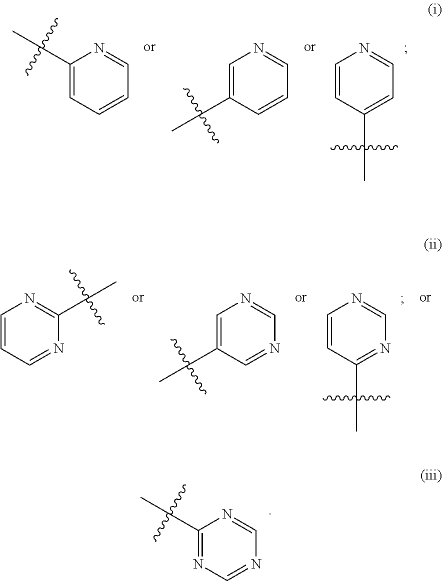

50. A method of preventing gastrointestinal bacterial invasion in a subject, comprising administering to the subject an effective amount of one or more compounds chosen from:
ethyl 5-[3-(trifluoromethyl)phenyl]-1H-pyrazole-3-carboxylate;
3,3-dimethyl-2-oxobutyl 5-(4-bromophenyl)-1H-pyrazole-3-carboxylate;
1-(tert-butylamino)-1-oxopropan-2-yl 5-(4-bromophenyl)-1H-pyrazole-3-carboxylate;
3-(2-hydroxyphenyl)-1H-pyrazole-5-carboxylic acid;
3-(4-isopropylphenyl)-1H-pyrazole-5-carboxylic acid;
methyl 3-(2,4-dichlorophenyl)-1H-pyrazole-5-carboxylate;
methyl 3-(2,4-dimethylphenyl)-1H-pyrazole-5-carboxylate;
3-(4-methoxyphenyl)-4-methylpyrano[2,3-c]pyrazol-6(1H)-one;
3-(4-methoxyphenyl)-4-methylpyrano[2,3-c]pyrazol-6-ol;
4-methyl-3-phenylpyrano[2,3-c]pyrazol-6-ol;
2-(1H-1,2,4-triazol-5-yl)-hexahydro-1H-isoindole-1,3(2H)-dione;
2-(1H-1,2,4-triazol-5-yl)-3a,4,7,7a-tetrahydro-1H-isoindole-1,3(2H)-dione;
N-[5-(4-bromobenzylthio)-4H-1,2,4-triazol-3-yl)acetamide; and
4-[(1-methyl-4H-imidazol-2-yl)methyl]-N-phenyl-1,3,5-triazin-2-amine.

51. A method for increasing the amount of intestinal alkaline phosphatase in a cell in vivo, in vitro, and ex vivo, comprising contacting a cell with an effective amount of one or more compounds chosen from:
ethyl 5-[3-(trifluoromethyl)phenyl]-1H-pyrazole-3-carboxylate;
3,3-dimethyl-2-oxobutyl 5-(4-bromophenyl)-1H-pyrazole-3-carboxylate;
1-(tert-butylamino)-1-oxopropan-2-yl 5-(4-bromophenyl)-1H-pyrazole-3-carboxylate;
3-(2-hydroxyphenyl)-1H-pyrazole-5-carboxylic acid;
3-(4-isopropylphenyl)-1H-pyrazole-5-carboxylic acid;
methyl 3-(2,4-dichlorophenyl)-1H-pyrazole-5-carboxylate;
methyl 3-(2,4-dimethylphenyl)-1H-pyrazole-5-carboxylate;
3-(4-methoxyphenyl)-4-methylpyrano[2,3-c]pyrazol-6(1H)-one;
3-(4-methoxyphenyl)-4-methylpyrano[2,3-c]pyrazol-6-ol;
4-methyl-3-phenylpyrano[2,3-c]pyrazol-6-ol;
2-(1H-1,2,4-triazol-5-yl)-hexahydro-1H-isoindole-1,3(2H)-dione;
2-(1H-1,2,4-triazol-5-yl)-3a,4,7,7a-tetrahydro-1H-isoindole-1,3(2H)-dione;
N-[5-(4-bromobenzylthio)-4H-1,2,4-triazol-3-yl)acetamide; and
4-[(1-methyl-4H-imidazol-2-yl)methyl]-N-phenyl-1,3,5-triazin-2-amine.

52. A method for activating intestinal alkaline phosphatase in a subject, comprising administering to the subject an effective amount of one or more compounds chosen from:
ethyl 5-[3-(trifluoromethyl)phenyl]-1H-pyrazole-3-carboxylate;
3,3-dimethyl-2-oxobutyl 5-(4-bromophenyl)-1H-pyrazole-3-carboxylate;
1-(tert-butylamino)-1-oxopropan-2-yl 5-(4-bromophenyl)-1H-pyrazole-3-carboxylate;
3-(2-hydroxyphenyl)-1H-pyrazole-5-carboxylic acid;
3-(4-isopropylphenyl)-1H-pyrazole-5-carboxylic acid;
methyl 3-(2,4-dichlorophenyl)-1H-pyrazole-5-carboxylate;
methyl 3-(2,4-dimethylphenyl)-1H-pyrazole-5-carboxylate;
3-(4-methoxyphenyl)-4-methylpyrano[2,3-c]pyrazol-6(1H)-one;
3-(4-methoxyphenyl)-4-methylpyrano[2,3-c]pyrazol-6-ol;
4-methyl-3-phenylpyrano[2,3-c]pyrazol-6-ol;
2-(1H-1,2,4-triazol-5-yl)-hexahydro-1H-isoindole-1,3(2H)-dione;
2-(1H-1,2,4-triazol-5-yl)-3a,4,7,7a-tetrahydro-1H-isoindole-1,3(2H)-dione;
N-[5-(4-bromobenzylthio)-4H-1,2,4-triazol-3-yl)acetamide; and
4-[(1-methyl-4H-imidazol-2-yl)methyl]-N-phenyl-1,3,5-triazin-2-amine.

53. A method for increasing the amount of alkaline phosphatase in a subject, comprising administering to a subject an effective amount of one or more compounds chosen from:
ethyl 5-[3-(trifluoromethyl)phenyl]-1H-pyrazole-3-carboxylate;
3,3-dimethyl-2-oxobutyl 5-(4-bromophenyl)-1H-pyrazole-3-carboxylate;
1-(tert-butylamino)-1-oxopropan-2-yl 5-(4-bromophenyl)-1H-pyrazole-3-carboxylate;
3-(2-hydroxyphenyl)-1H-pyrazole-5-carboxylic acid;
3-(4-isopropylphenyl)-1H-pyrazole-5-carboxylic acid;
methyl 3-(2,4-dichlorophenyl)-1H-pyrazole-5-carboxylate;
methyl 3-(2,4-dimethylphenyl)-1H-pyrazole-5-carboxylate;
3-(4-methoxyphenyl)-4-methylpyrano[2,3-c]pyrazol-6(1H)-one;
3-(4-methoxyphenyl)-4-methylpyrano[2,3-c]pyrazol-6-ol;
4-methyl-3-phenylpyrano[2,3-c]pyrazol-6-ol;
